(19) United States
(12) Patent Application Publication
(10) Pub. No.: US 2015/0119394 A1
(43) Pub. Date: Apr. 30, 2015

(54) Insulin-Like Growth Factor-1 Receptor Inhibitors

(71) Applicants: Merck Sharp & Dohme Corp., Rahway, NJ (US); Piramal Enterprises Limited, Mumbai (IN)

(72) Inventors: Abhijit Roychowdhury, Mumbai (IN); Rajiv Sharma, Mumbai (IN); Malcolm Mascarenhas, Mumbai (IN); Chandrika B-Rao, Mumbai (IN); Shashikant M. Patil, Mumbai (IN); Sambhaji Chavan, Mumbai (IN); Nitin Lad, Mumbai (IN); Smriti Khanna, Mumbai (IN); Christopher J. Dinsmore, Boston, MA (US)

(21) Appl. No.: 14/387,935

(22) PCT Filed: Mar. 14, 2013
(86) PCT No.: PCT/US13/31155

§ 371 Date: Sep. 25, 2014

Related U.S. Application Data
(60) Provisional application No. 61/616,691, filed on Mar. 28, 2012.

(51) Int. Cl. C07D 413/12; C07D 413/14

(52) U.S. Cl. CPC C07D 413/12; C07D 413/14
USPC 514/234.5; 544/144; 514/235.2; 544/124; 544/123; 544/122; 544/120; 544/119; 544/128

(57) ABSTRACT

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety. The present invention is also related to the pharmaceutically acceptable salts, hydrates and stereoisomers of these compounds.

### TECHNICAL FIELD OF INVENTION

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety.

### BACKGROUND OF THE INVENTION

Protein kinases (PKs) are enzymes that catalyze the phosphorylation of hydroxy groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non life-threatening diseases such as psoriasis to extremely virulent diseases such as glioblastoma (brain cancer). PKs can be broken into two classes, the protein tyrosine kinases (PTKs) and the serine-threonine kinases (STKs).

Certain growth factor receptors exhibiting PK activity are known as receptor tyrosine kinases (RTKs). They comprise a large family of transmembrane receptors with diverse biological activity. At present, at least nineteen (19) distinct subfamilies of RTKs have been identified. One RTK subfamily contains the insulin receptor (IR), insulin-like growth factor I receptor (IGF-1R) and insulin receptor related receptor (IRR). IR and IGF-1R interact with insulin to activate a hetero-tetramer composed of two entirely extracellular glycosylated α subunits and two β subunits which cross the cell membrane and which contain the tyrosine kinase domain. The Insulin-like Growth Factor-1 Receptor (IGF-1R), and its ligands, IGF-1 and IGF-2, are abnormally expressed in numerous tumors, including, but not limited to, breast, prostate, thyroid, lung, hepatoma, colon, brain, neuroendocrine, and others.

Numerous IGF-1R small molecule inhibitors have been found to inhibit cancer growth in vitro, in vivo and in clinical trials. For example, BMS-754807 effectively inhibits the growth of a broad range of human tumor types in vitro, including mesenchymal (Ewing's, rhabdomyosarcoma, neuroblastoma, and liposarcoma), epothelial (breast, lung, pancreatic, colon, gastric), and hematopoietic (multiple myeloma and leukemia) tumor cell lines. Carboni et al., Mol Cancer Ther 2009; 8(12).

### SUMMARY OF THE INVENTION

The present invention relates to compounds that are capable of inhibiting, modulating and/or regulating Insulin-Like-Growth Factor I Receptor and Insulin Receptor. The compounds of the instant invention possess a core structure that comprises a sulfonyl indole moiety. The present invention is also related to the pharmaceutically acceptable salts, hydrates and stereoisomers of these compounds.

### DETAILED DESCRIPTION OF THE INVENTION

The representative compounds of this invention are useful in the inhibition of IGF-1R or IR and are illustrated by a compound of Formula I:

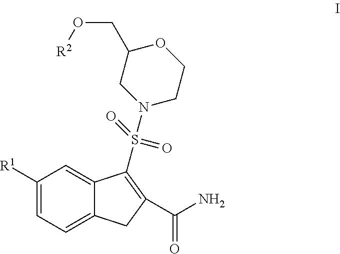

wherein:
R¹ is H, halo, or CN;

### R² is

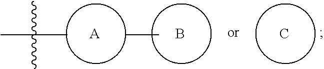

Ring A is phenyl or a 5 or 6-membered heteroaryl, which can be optionally substituted with one to three moieties selected from C₁-C₃ alkyl, C₁-C₃ haloalkyl, OR³, N(R^{a})₂ and halo;
Ring B is phenyl or a 5 or 6-membered heteroaryl, which can be optionally substituted with one to three moieties selected from C₁-C₃ alkyl, C₁-C₃ haloalkyl, OR³, N(R^{a})₂ and halo;
Ring C is a 10-membered fused bicyclic aryl or heteroaryl, which can be optionally substituted with one to three moieties selected from C₁-C₃ alkyl and halo;
R^{a} is independently H or C₁-C₃ alkyl;
R³ is H, C₁-C₃ haloalkyl or C₁-C₃ alkyl;
or a pharmaceutically acceptable salt thereof.

In one embodiment of the invention, R² is

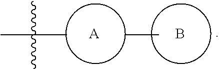

In another embodiment of the invention, Ring A is phenyl, pyridyl, pyrazinyl, or pyrazolyl; Ring B is phenyl, pyridyl, or pyrimidinyl. In a further embodiment of the invention, Ring A is phenyl; Ring B is phenyl or pyridyl.

In another embodiment of the invention, Ring C is naphthyl, cinnolinyl, quinolinyl, or quinazolinyl.

The present invention also provides compounds under formula IA:

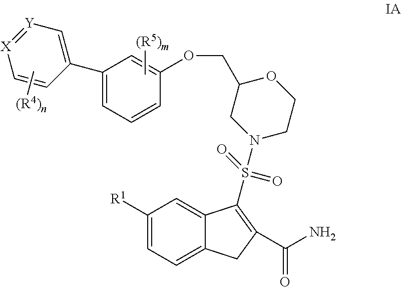

### Wherein

### X is C or N;

Y is C or N; provided that X and Y are not both N;
R¹ is halo;
R⁴ is H, C₁-C₃ alkyl, C₁-C₃ haloalkyl, OR³, N(R^{a})₂ or halo;
R⁵ is H, C₁-C₃ alkyl, C₁-C₃ haloalkyl, OR³, N(R^{a})₂ or halo;
R^{a} is independently H or C₁-C₃ alkyl; and
R³ is H, C₁-C₃ haloalkyl or C₁-C₃ alkyl;
n is 0, 1, 2 or 3; and
m is 0, 1, 2 or 3.

The present invention also provides compounds under formula IB:

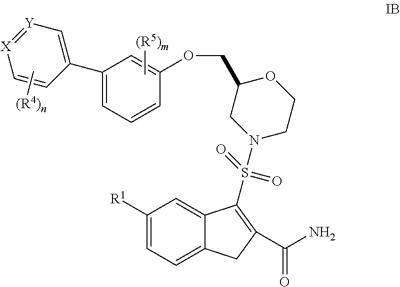

Wherein all substituents are defined as above.

In one embodiment of formula IA or IB,

Y is N and X is C or X and Y are both C;

### R¹ is Cl;

R⁴ is H, methyl, methoxy or halo;
R⁵ is H, methyl, methoxy or halo;
n is 0 or 1; and
m is 0 or 1.

In one embodiment of formula IA or IB,

Y is N and X is C or X and Y are both C;

### R¹ is Cl;

R⁴ is H or methoxy;
R⁵ is H or methoxy;
n is 0 or 1; and
m is 0 or 1.

In one embodiment under formula IA or IB, Y is N and X is C. In another embodiment, X and Y are both C.

The present invention also provides compounds under formula IIA:

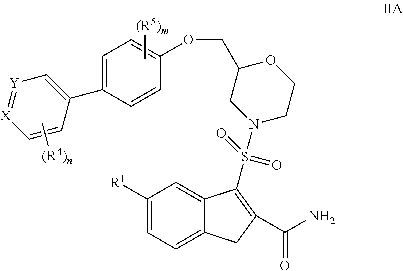

### Wherein

### X is C or N;

Y is C or N; provided that X and Y are not both N;
R¹ is halo;
R⁴ is H, C₁-C₃ alkyl, C₁-C₃ haloalkyl, OR³, N(R^{a})₂ or halo;
R⁵ is H, C₁-C₃ alkyl, C₁-C₃ haloalkyl, OR³, N(R^{a})₂ or halo;
R^{a} is independently H or C₁-C₃ alkyl; and
R³ is H, C₁-C₃ haloalkyl or C₁-C₃ alkyl;
n is 0, 1, 2 or 3; and
m is 0, 1, 2 or 3.

The present invention also provides compounds under formula IIB

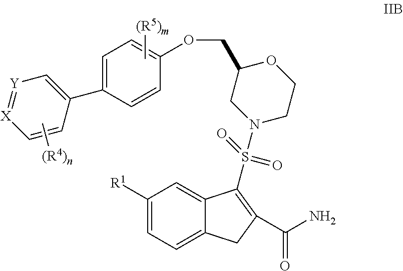

Wherein all other substituents are as defined above.

In one embodiment of formula IIA or IIB,

### Y is C and X is N;

### R¹ is Cl;

R⁴ is H, methyl or halo;
R⁵ is H, methyl or halo;
n is 0 or 1; and
m is 0 or 1.

In another embodiment under formula IA, IB, IIA or IIB, R⁴ is H, methyl or fluoro; R⁵ is H, methyl or fluoro.

Specific Examples of the compounds of the invention are:
(S)-3-((2-(([1,1′-biphenyl]-3-yloxy)methyl)morpholino)sulfonyl)-5-chloro-1H-indole-2-carboxamide;
(S)-5-chloro-3-((2-(3-(pyridin-4-yl)phenoxy)methyl)morpholino)sulfonyl)-1H-indole-2-carboxamide;
(S)-5-chloro-3-((2-(3-(2-fluoropyridin-3-yl)phenoxy)methyl)morpholino)sulfonyl)-1H-indole-2-carboxamide;
(S)-5-chloro-3-((2-(3-(6-methylpyridin-3-yl)phenoxy)methyl)morpholino)sulfonyl)-1H-indole-2-carboxamide;
(S)-5-chloro-3-((2-(3-(6-(dimethylamino)pyridin-3-yl)phenoxy)methyl)morpholino)sulfonyl)-1H-indole-2-carboxamide;
(S)-5-chloro-3-((2-(3-(6-(trifluoromethyl)pyridin-3-yl)phenoxy)methyl)morpholino)sulfonyl)-1H-indole-2-carboxamide;
(S)-5-chloro-3-((2-(3-(6-methoxypyridin-3-yl)phenoxy)methyl)morpholino)sulfonyl)-1H-indole-2-carboxamide;
(S)-5-chloro-3-((2-(3-(6-fluoropyridin-3-yl)phenoxy)methyl)morpholino)sulfonyl)-1H-indole-2-carboxamide;
(S)-5-chloro-3-((2-(((2′-fluoro-[1,1′-biphenyl]-4-yl)oxy)methyl)morpholino)sulfonyl)-1H-indole-2-carboxamide;
(S)-5-chloro-3-((2-(((3′-(dimethylamino)-[1,1′-biphenyl]-3-yl)oxy)methyl)morpholino)sulfonyl)-1H-indole-2-carboxamide;
(S)-5-chloro-3-((2-(((2′-methyl-[1,1′-biphenyl]-3-yl)oxy)methyl)morpholino)sulfonyl)-1H-indole-2-carboxamide;
(S)-5-chloro-3-((2-(((2′-methoxy-[1,1′-biphenyl]-4-yl)oxy)methyl)morpholino)sulfonyl)-1H-indole-2-carboxamide;
(S)-5-chloro-3-((2-((4-(2-methoxypyridin-3-yl)phenoxy)methyl)morpholino)sulfonyl)-1H-indole-2-carboxamide;
(S)-5-chloro-3-((2-(((4′-fluoro-[1,1′-biphenyl]-3-yl)oxy)methyl)morpholino)sulfonyl)-1H-indole-2-carboxamide;
(S)-5-chloro-3-((2-(((3′-fluoro-[1,1′-biphenyl]-3-yl)oxy)methyl)morpholino)sulfonyl)-1H-indole-2-carboxamide;
(S)-5-chloro-3-((2-(3-(2-fluoropyridin-4-yl)phenoxy)methyl)morpholino)sulfonyl)-1H-indole-2-carboxamide;
(S)-5-chloro-3-((2-(((3′-methyl-[1,1′-biphenyl]-3-yl)oxy)methyl)morpholino)sulfonyl)-1H-indole-2-carboxamide;
(S)-5-chloro-3-((2-(3-(pyridin-2-yl)phenoxy)methyl)morpholino)sulfonyl)-1H-indole-2-carboxamide;
(S)-5-chloro-3-((2-((3-(2-methoxypyrimidin-5-yl)phenoxy)methyl)morpholino)sulfonyl)-1H-indole-2-carboxamide;
(S)-5-chloro-3-((2-(3-(6-ethoxypyridin-3-yl)phenoxy)methyl)morpholino)sulfonyl)-1H-indole-2-carboxamide;
(S)-5-chloro-3-((2-(3-(2-methoxypyridin-4-yl)phenoxy)methyl)morpholino)sulfonyl)-1H-indole-2-carboxamide;
(S)-5-chloro-3-((2-(3-(2-methylpyridin-4-yl)phenoxy)methyl)morpholino)sulfonyl)-1H-indole-2-carboxamide;
(S)-5-chloro-3-((2-(((6-methyl-[1,1′-biphenyl]-3-yl)oxy)methyl)morpholino)sulfonyl)-1H-indole-2-carboxamide;
(S)-5-chloro-3-((2-(((2′-fluoro-6-methyl-[1,1′-biphenyl]-3-yl)oxy)methyl)morpholino)sulfonyl)-1H-indole-2-carboxamide;
(S)-5-chloro-3-((2-(3-(2-fluoropyridin-3-yl)-4-methylphenoxy)methyl)morpholino)sulfonyl)-1H-indole-2-carboxamide;
(S)-5-chloro-3-((2-(((2′-methoxy-6-methyl-[1,1′-biphenyl]-3-yl)oxy)methyl)morpholino)sulfonyl)-1H-indole-2-carboxamide;
(S)-5-chloro-3-((2-(((2′,6-dimethyl-[1,1′-biphenyl]-3-yl)oxy)methyl)morpholino)sulfonyl)-1H-indole-2-carboxamide;
(S)-5-chloro-3-((2-(3-(6-methoxypyridin-3-yl)-4-methylphenoxy)methyl)morpholino)sulfonyl)-1H-indole-2-carboxamide;
(S)-5-chloro-3-((2-((4-fluoro-3-(pyridin-3-yl)phenoxy)methyl)morpholino)sulfonyl)-1H-indole-2-carboxamide;
(S)-5-chloro-3-((2-((4-fluoro-3-(pyrimidin-5-yl)phenoxy)methyl)morpholino)sulfonyl)-1H-indole-2-carboxamide;
(S)-5-chloro-3-((2-((4-fluoro-3-(pyridin-4-yl)phenoxy)methyl)morpholino)sulfonyl)-1H-indole-2-carboxamide;
(S)-5-chloro-3-((2-((4-fluoro-3-(6-methoxypyridin-3-yl)phenoxy)methyl)morpholino)sulfonyl)-1H-indole-2-carboxamide;
(S)-5-chloro-3-((2-((4-fluoro-3-(2-methylpyridin-4-yl)phenoxy)methyl)morpholino)sulfonyl)-1H-indole-2-carboxamide;
(S)-5-chloro-3-((2-((4-(pyridin-3-yl)phenoxy)methyl)morpholino)sulfonyl)-1H-indole-2-carboxamide;
(S)-5-chloro-3-((2-((4-(pyridin-4-yl)phenoxy)methyl)morpholino)sulfonyl)-1H-indole-2-carboxamide;
(S)-3-((2-(([1,1′-biphenyl]-4-yloxy)methyl)morpholino)sulfonyl)-5-chloro-1H-indole-2-carboxamide;
(S)-5-chloro-3-((2-((4-(6-(dimethylamino)pyridin-3-yl)phenoxy)methyl)morpholino)sulfonyl)-1H-indole-2-carboxamide;
(S)-5-chloro-3-((2-((4-(6-(trifluoromethyl)pyridin-3-yl)phenoxy)methyl)morpholino)sulfonyl)-1H-indole-2-carboxamide;
(S)-5-chloro-3-((2-(((2′-methoxy-[1,1′-biphenyl]-4-yl)oxy)methyl)morpholino)sulfonyl)-1H-indole-2-carboxamide;
(S)-5-chloro-3-((2-((4-(6-methoxypyridin-3-yl)phenoxy)methyl)morpholino)sulfonyl)-1H-indole-2-carboxamide;
(S)-5-chloro-3-((2-(((2′-(trifluoromethyl)-[1,1′-biphenyl]-4-yl)oxy)methyl)morpholino)sulfonyl)-1H-indole-2-carboxamide;
(S)-5-chloro-3-((2-(((4′-methoxy-[1,1′-biphenyl]-4-yl)oxy)methyl)morpholino)sulfonyl)-1H-indole-2-carboxamide;
(S)-5-chloro-3-((2-(((2′-(trifluoromethoxy)-[1,1′-biphenyl]-4-yl)oxy)methyl)morpholino)sulfonyl)-1H-indole-2-carboxamide;
(S)-5-chloro-3-((2-(((2′-methyl-[1,1′-biphenyl]-4-yl)oxy)methyl)morpholino)sulfonyl)-1H-indole-2-carboxamide;
(S)-5-chloro-3-((2-((4-(2-fluoropyridin-4-yl)phenoxy)methyl)morpholino)sulfonyl)-1H-indole-2-carboxamide;
(S)-5-chloro-3-((2-((4-(6-fluoropyridin-3-yl)phenoxy)methyl)morpholino)sulfonyl)-1H-indole-2-carboxamide;
(S)-5-chloro-3-((2-((4-(2-methoxypyridin-4-yl)phenoxy)methyl)morpholino)sulfonyl)-1H-indole-2-carboxamide;
(S)-5-chloro-3-((2-((4-(pyrimidin-5-yl)phenoxy)methyl)morpholino)sulfonyl)-1H-indole-2-carboxamide;
(S)-5-chloro-3-((2-((4-(2-fluoropyridin-3-yl)phenoxy)methyl)morpholino)sulfonyl)-1H-indole-2-carboxamide;
(S)-5-chloro-3-((2-((4-(2-methylpyridin-3-yl)phenoxy)methyl)morpholino)sulfonyl)-1H-indole-2-carboxamide;
(S)-5-chloro-3-((2-((4-(2-methylpyridin-4-yl)phenoxy)methyl)morpholino)sulfonyl)-1H-indole-2-carboxamide;
(S)-5-chloro-3-((2-((4-(6-methylpyridin-3-yl)phenoxy)methyl)morpholino)sulfonyl)-1H-indole-2-carboxamide;
(S)-5-chloro-3-((2-((4-(pyrimidin-2-yl)phenoxy)methyl)morpholino)sulfonyl)-1H-indole-2-carboxamide;
(S)-3-((2-(([1,1′-biphenyl]-2-yloxy)methyl)morpholino)sulfonyl)-5-chloro-1H-indole-2-carboxamide;
(S)-5-chloro-3-((2-((2-(pyridin-3-yl)phenoxy)methyl)morpholino)sulfonyl)-1H-indole-2-carboxamide;
(S)-5-chloro-3-((2-((2-(pyridin-4-yl)phenoxy)methyl)morpholino)sulfonyl)-1H-indole-2-carboxamide;
(S)-5-chloro-3-((2-((2-(2-fluoropyridin-3-yl)phenoxy)methyl)morpholino)sulfonyl)-1H-indole-2-carboxamide;
(S)-5-chloro-3-((2-(((2′-methoxy-[1,1′-biphenyl]-2-yl)oxy)methyl)morpholino)sulfonyl)-1H-indole-2-carboxamide;
(S)-5-chloro-3-((2-((2-(6-fluoropyridin-3-yl)phenoxy)methyl)morpholino)sulfonyl)-1H-indole-2-carboxamide;
(S)-5-chloro-3-((2-((2-(pyrimidin-5-yl)phenoxy)methyl)morpholino)sulfonyl)-1H-indole-2-carboxamide;
(S)-5-chloro-3-((2-((2-(6-methoxypyridin-3-yl)phenoxy)methyl)morpholino)sulfonyl)-1H-indole-2-carboxamide;
(S)-5-chloro-3-((2-(((2′-fluoro-[1,1′-biphenyl]-2-yl)oxy)methyl)morpholino)sulfonyl)-1H-indole-2-carboxamide;
(S)-5-chloro-3-((2-((2-(2-methylpyridin-3-yl)phenoxy)methyl)morpholino)sulfonyl)-1H-indole-2-carboxamide;
(S)-5-chloro-3-((2-(((3′-fluoro-[1,1′-biphenyl]-2-yl)oxy)methyl)morpholino)sulfonyl)-1H-indole-2-carboxamide;
(S)-5-chloro-3-((2-(((2′-fluoro-4′-(trifluoromethyl)-[1,1′-biphenyl]-2-yl)oxy)methyl)morpholino)sulfonyl)-1H-indole-2-carboxamide;
(S)-5-chloro-3-((2-((3-methyl-4-(pyridin-3-yl)phenoxy)methyl)morpholino)sulfonyl)-1H-indole-2-carboxamide;
(S)-5-chloro-3-((2-((3-methyl-4-(pyridin-4-yl)phenoxy)methyl)morpholino)sulfonyl)-1H-indole-2-carboxamide;
(S)-5-chloro-3-((2-((4-(2-fluoropyridin-3-yl)-3-methylphenoxy)methyl)morpholino)sulfonyl)-1H-indole-2-carboxamide;
(S)-5-chloro-3-((2-((4-(6-methoxypyridin-3-yl)-3-methylphenoxy)methyl)morpholino)sulfonyl)-1H-indole-2-carboxamide;
(S)-5-chloro-3-((2-((4-(2-methoxypyridin-4-yl)-3-methylphenoxy)methyl)morpholino)sulfonyl)-1H-indole-2-carboxamide;
(S)-5-chloro-3-((2-((3-methyl-4-(pyrimidin-5-yl)phenoxy)methyl)morpholino)sulfonyl)-1H-indole-2-carboxamide;
(S)-5-chloro-3-((2-((4-(2-fluoropyridin-4-yl)-3-methylphenoxy)methyl)morpholino)sulfonyl)-1H-indole-2-carboxamide; (S)-5-chloro-3-((2-((4-(6-fluoropyridin-3-yl)-3-methylphenoxy)methyl)morpholino)sulfonyl)-1H-indole-2-carboxamide;
(S)-5-chloro-3-((2-(((5-(4-methoxyphenyl)pyridin-3-yl)oxy)methyl)morpholino)sulfonyl)-1H-indole-2-carboxamide;
(S)-5-chloro-3-((2-(((5-(2-fluoro-5-(trifluoromethyl)phenyl)pyridin-3-yl)oxy)methyl)morpholino)sulfonyl)-1H-indole-2-carboxamide;
(S)-5-chloro-3-((2-(((5-phenylpyridin-3-yl)oxy)methyl)morpholino)sulfonyl)-1H-indole-2-carboxamide;
(S)-5-chloro-3-((2-(((5-phenylpyrazin-2-yl)oxy)methyl)morpholino)sulfonyl)-1H-indole-2-carboxamide;
(S)-5-chloro-3-((2-(((5-(2-fluorophenyl)pyridin-3-yl)oxy)methyl)morpholino)sulfonyl)-1H-indole-2-carboxamide;
(S)-5-chloro-3-((2-(((6-phenylpyridin-3-yl)oxy)methyl)morpholino)sulfonyl)-1H-indole-2-carboxamide;
(S)-5-chloro-3-((2-(((6-phenylpyridin-2-yl)oxy)methyl)morpholino)sulfonyl)-1H-indole-2-carboxamide;
(S)-5-chloro-3-((2-(((5-phenylpyridin-2-yl)oxy)methyl)morpholino)sulfonyl)-1H-indole-2-carboxamide;
(S)-5-chloro-3-((2-((4-(2,6-dimethylpyridin-4-yl)phenoxy)methyl)morpholino)sulfonyl)-1H-indole-2-carboxamide;
(S)-5-chloro-3-((2-(((2′-chloro-[1,1′-biphenyl]-4-yl)oxy)methyl)morpholino)sulfonyl)-1H-indole-2-carboxamide;
(S)-5-chloro-3-((2-((4-(2-chloropyridin-4-yl)phenoxy)methyl)morpholino)sulfonyl)-1H-indole-2-carboxamide;
(S)-5-chloro-3-((2-(3-(2-chloropyridin-4-yl)phenoxy)methyl)morpholino)sulfonyl)-1H-indole-2-carboxamide;
(S)-5-chloro-3-((2-(3-(6-chloropyridin-3-yl)phenoxy)methyl)morpholino)sulfonyl)-1H-indole-2-carboxamide;
(S)-5-chloro-3-((2-((4-(2-chloropyridin-4-yl)-3-methylphenoxy)methyl)morpholino)sulfonyl)-1H-indole-2-carboxamide;
(S)-5-chloro-3-((2-((4-(6-chloropyridin-3-yl)-3-methylphenoxy)methyl)morpholino)sulfonyl)-1H-indole-2-carboxamide;
(S)-5-chloro-3-((2-((cinnolin-3-yloxy)methyl)morpholino)sulfonyl)-1H-indole-2-carboxamide;
(S)-5-chloro-3-((2-((quinolin-8-yloxy)methyl)morpholino)sulfonyl)-1H-indole-2-carboxamide;
(S)-5-chloro-3-((2-((quinazolin-4-yloxy)methyl)morpholino)sulfonyl)-1H-indole-2-carboxamide; and
(S)-5-chloro-3-((2-(((1-methyl-3-phenyl-1H-pyrazol-5-yl)oxy)methyl)morpholino)sulfonyl)-1H-indole-2-carboxamide; or a stereoisomer thereof;
   or a pharmaceutically acceptable salt thereof;
   or a pharmaceutically acceptable salt of the stereoisomer thereof.

### Chemical Definitions

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylaryl”, “alkylcycloalkyl” and “alkylheterocyclyl” the term “alkyl” refers to the alkyl portion of the moiety and does not describe the number of atoms in the heterocyclyl portion of the moiety. In an embodiment, if the number of carbon atoms is not specified, the “alkyl” of “alkylaryl”, “alkylcycloalkyl” and “alkylheterocyclyl” refers to C₁-C₁₂ alkyl and in a further embodiment, refers to C₁-C₆ alkyl.

The term “cycloalkyl” means a monocyclic saturated or unsaturated aliphatic hydrocarbon group having the specified number of carbon atoms. The cycloalkyl is optionally bridged (i.e., forming a bicyclic moiety), for example with a methylene, ethylene or propylene bridge. The cycloalkyl may be fused with an aryl group such as phenyl, and it is understood that the cycloalkyl substituent is attached via the cycloalkyl group. For example, “cycloalkyl” includes cyclopropyl, methyl-cyclopropyl, 2,2-dimethyl-cyclobutyl, 2-ethyl-cyclopentyl, cyclohexyl, cyclopentenyl, cyclobutenyl and so on.

In an embodiment, if the number of carbon atoms is not specified, “alkyl” refers to C₁-C₁₂ alkyl and in a further embodiment, “alkyl” refers to C₁-C₆ alkyl. In an embodiment, if the number of carbon atoms is not specified, “cycloalkyl” refers to C₃-C₁₀ cycloalkyl and in a further embodiment, “cycloalkyl” refers to C₃-C₇ cycloalkyl. In an embodiment, examples of “alkyl” include methyl, ethyl, n-propyl, i-propyl, n-butyl, t-butyl and i-butyl.

The term “alkylene” means a hydrocarbon diradical group having the specified number of carbon atoms. For example, “alkylene” includes —CH₂—, —CH₂CH₂— and the like. In an embodiment, if the number of carbon atoms is not specified, “alkylene” refers to C₁-C₁₂ alkylene and in a further embodiment, “alkylene” refers to C₁-C₆ alkylene.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

“Alkenylene” means a diradical group of an alkenyl group that is defined above. For example, “alkenylene” includes —CH₂—CH₂—CH═CH—CH₂, —CH═CH—CH₂ and the like.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

“Aryl” is intended to mean any stable monocyclic, bicyclic or tricyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In one embodiment, “aryl” is an aromatic ring of 6 to 14 carbons atoms, and includes a carbocyclic aromatic group fused with a 5-or 6-membered cycloalkyl group such as indan. Examples of carbocyclic aromatic groups include, but are not limited to, phenyl, naphthyl, e.g. 1-naphthyl and 2-naphthyl; anthracenyl, e.g. 1-anthracenyl, 2-anthracenyl; phenanthrenyl; fluorenonyl, e.g. 9-fluorenonyl, indanyl and the like.

The term heteroaryl, as used herein, represents a stable monocyclic, bicyclic or tricyclic ring of up to 7 atoms in each ring, wherein at least one ring is aromatic and contains carbon and from 1 to 4 heteroatoms selected from the group consisting of O, N and S. In another embodiment, the term heteroaryl refers to a monocyclic, bicyclic or tricyclic aromatic ring of 5- to 14-ring atoms of carbon and from one to four heteroatoms selected from O, N, or S. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. Additional examples of heteroaryl include, but are not limited to pyridyl, e.g., 2-pyridyl (also referred to as α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like.

In an embodiment, “heteroaryl” may also include a “fused polycyclic aromatic”, which is a heteroaryl fused with one or more other heteroaryl or nonaromatic heterocyclic ring. Examples include, quinolinyl and isoquinolinyl, e.g. 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g. 2-benzofuranyl and 3-benzofuranyl; dibenzofuranyl, e.g. 2,3-dihydrobenzofuranyl; dibenzothiophenyl; benzothienyl, e.g. 2-benzothienyl and 3-benzothienyl; indolyl, e.g. 2-indolyl and 3-indolyl; benzothiazolyl, e.g., 2-benzothiazolyl; benzooxazolyl, e.g., 2-benzooxazolyl; benzimidazolyl, e.g. 2-benzoimidazolyl; isoindolyl, e.g. 1-isoindolyl and 3-isoindolyl; benzotriazolyl; purinyl; thianaphthenyl, pyrazinyl and the like.

“Heterocyclyl” means a non-aromatic saturated monocyclic, bicyclic, tricyclic or spirocyclic ring system comprising up to 7 atoms in each ring. Preferably, the heterocyclyl contains 3 to 14, or 5 to 10 ring atoms, in which one or more of the atoms in the ring system is an element other than carbon, for example, nitrogen, oxygen, phosphor or sulfur, alone or in combination. There are no adjacent oxygen and/or sulfur atoms present in the ring system. Preferred heterocyclyls contain about 5 to about 6 ring atoms. The heterocycle may be fused with an aromatic aryl group such as phenyl or heterocyclenyl. The prefix aza, oxa or thia before the heterocyclyl root name means that at least a nitrogen, oxygen or sulfur atom, respectively, is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone:

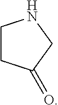

In describing the heteroatoms contained in a specified heterocyclyl group, the expression, “having one to x heteroatoms selected from the group of N, O, P and S” (wherein x is an a specified integer), for example, means that each heteroatom in the specified heterocyclyl is independently selected from the specified selection of heteroatoms. Attachment of a heterocyclyl substituent can occur via a carbon atom or via a heteroatom.

“Heterocyclenyl” means a non-aromatic monocyclic, bicyclic, tricyclic or spirocyclic ring system comprising up to 7 atoms in each ring. Preferably, the heterocyclenyl contains 3 to 14, or 5 to 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen, phosphor or sulfur atom respectively is present as a ring atom. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. An example of such a moiety is pyrrolidinone:

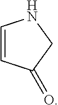

In describing the heteroatoms contained in a specified heterocyclenyl group, the expression, “having one to x heteroatoms selected from the group of N, O, P and S” (wherein x is an a specified integer), for example, means that each heteroatom in the specified heterocyclenyl is independently selected from the specified selection of heteroatoms.

It should also be noted that tautomeric forms such as, for example, the moieties:

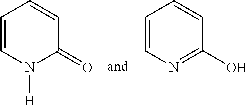 [SMILES: O=c1cccc[nH]1 ; Oc1ccccn1]

are considered equivalent in certain embodiments of this invention.

An “alkylaryl group” is an alkyl group substituted with an aryl group, for example, a phenyl group. Suitable aryl groups are described herein and suitable alkyl groups are described herein. The bond to the parent moiety is through the aryl group.

An “alkylheteroaryl group” is an alkyl group substituted with a heteroaryl group. Suitable heteroaryl groups are described herein and suitable alkyl groups are described herein. The bond to the parent moiety is through the heteroaryl group.

An “alkylheterocyclyl group” is an alkyl group substituted with a heterocyclyl group. Suitable heterocyclyl groups are described herein and suitable alkyl groups are described herein. The bond to the parent moiety is through the heterocyclyl group.

An “alkylheterocyclenyl group” is an alkyl group substituted with a heterocyclenyl group. Suitable heterocyclenyl groups are described herein and suitable alkyl groups are described herein. The bond to the parent moiety is through the heterocyclenyl group.

An “alkylcycloalkyl group” is an alkyl group substituted with a cycloalkyl group. Suitable cycloalkyl groups are described herein and suitable alkyl groups are described herein. The bond to the parent moiety is through the cycloalkyl group.

An “arylalkyl group” is an aryl group substituted with an alkyl group. Suitable aryl groups are described herein and suitable alkyl groups are described herein. The bond to the parent moiety is through the alkyl group.

A “heteroarylalkyl group” is a heteroaryl group substituted with an alkyl group. Suitable heteroaryl groups are described herein and suitable alkyl groups are described herein. The bond to the parent moiety is through the alkyl group.

A “heterocyclylalkyl group” is a heterocyclyl group substituted with an alkyl group. Suitable heterocyclyl groups are described herein and suitable alkyl groups are described herein. The bond to the parent moiety is through the alkyl group.

A “heterocyclenylalkyl group” is a heterocyclenyl group substituted with an alkyl group. Suitable heterocyclenyl groups are described herein and suitable alkyl groups are described herein. The bond to the parent moiety is through the alkyl group.

A “cycloalkylalkyl group” is a cycloalkyl group substituted with an alkyl group. Suitable cycloalkyl groups are described herein and suitable alkyl groups are described herein. The bond to the parent moiety is through the alkyl group.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

An “alkylamino group” as used herein, is an alkyl group that is attached to a compound via a nitrogen.

As used herein, an “arylalkylamino group” is an arylalkyl group that is attached to a compound via a nitrogen on the alkyl portion of the arylalkyl.

An “alkylsulfonyl group” as used herein, is an alkyl group that is attached to a compound via the sulfur of a sulfonyl group.

When a moiety is referred to as “unsubstituted” or not referred to as “substituted” or “optionally substituted”, it means that the moiety does not have any substituents. When a moiety is referred to as substituted, it denotes that any portion of the moiety that is known to one skilled in the art as being available for substitution can be substituted. The phrase “optionally substituted with one or more substituents” means, in one embodiment, one substituent, two substituents, three substituents, four substituents or five substituents. For example, the substitutable group can be a hydrogen atom that is replaced with a group other than hydrogen (i.e., a substituent group). Multiple substituent groups can be present. When multiple substituents are present, the substituents can be the same or different and substitution can be at any of the substitutable sites. Such means for substitution are well known in the art. For purposes of exemplification, which should not be construed as limiting the scope of this invention, some examples of groups that are substituents are: alkyl, alkenyl or alkynyl groups (which can also be substituted, with one or more substituents), alkoxy groups (which can be substituted), a halogen or halo group (F, Cl, Br, I), hydroxy, nitro, oxo, —CN, —COH, —COOH, amino, azido, N-alkylamino or N,N-dialkylamino (in which the alkyl groups can also be substituted), N-arylamino or N,N-diarylamino (in which the aryl groups can also be substituted), esters (—C(O)—OR, where R can be a group such as alkyl, aryl, etc., which can be substituted), ureas (—NHC(O)—NHR, where R can be a group such as alkyl, aryl, etc., which can be substituted), carbamates (—NHC(O)—OR, where R can be a group such as alkyl, aryl, etc., which can be substituted), sulfonamides (—NHS(O)₂R, where R can be a group such as alkyl, aryl, etc., which can be substituted), alkylsulfonyl (which can be substituted), aryl (which can be substituted), cycloalkyl (which can be substituted) alkylaryl (which can be substituted), alkylheterocyclyl (which can be substituted), alkylcycloalkyl (which can be substituted), and aryloxy.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

As used herein, the term “composition” is intended to encompass a product comprising the specified ingredients in the specified amounts, as well as any product which results, directly or indirectly, from combination of the specified ingredients in the specified amounts.

### Isotopes

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. For instance those compounds labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Additionally, isotopic substitution of a compound at a site where epimerization occurs may slow or reduce the epimerization process and thereby retain the more active or efficacious form of the compound for a longer period of time.

### Stereochemistry

When structures of the same constitution differ in respect to the spatial arrangement of certain atoms or groups, they are stereoisomers, and the considerations that are significant in analyzing their interrelationships are topological. If the relationship between two stereoisomers is that of an object and its nonsuperimposable mirror image, the two structures are enantiomeric, and each structure is said to be chiral. Stereoisomers also include diastereomers, cis-trans isomers and conformational isomers. Diastereoisomers can be chiral or achiral, and are not mirror images of one another. Cis-trans isomers differ only in the positions of atoms relative to a specified planes in cases where these atoms are, or are considered as if they were, parts of a rigid structure. Conformational isomers are isomers that can be interconverted by rotations about formally single bonds. Examples of such conformational isomers include cyclohexane conformations with chair and boat conformers, carbohydrates, linear alkane conformations with staggered, eclipsed and gauche conformers, etc. See J. Org. Chem. 35, 2849 (1970).

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, enantiomers are identical except that they are non-superimposable mirror images of one another. A mixture of enantiomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the Formulas of the invention, it is understood that both the (R) and (S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the compounds of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

### Solvates

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The active compounds disclosed can also be prepared in any solid or liquid physical form. For example, the compound can be in a crystalline form, in amorphous form, and have any particle size. Furthermore, the compound particles may be micronized, or may be agglomerated, particulate granules, powders, oils, oily suspensions or any other form of solid or liquid physical form.

The compounds of the present invention may also exhibit polymorphism. This invention further includes different polymorphs of the compounds of the present invention. The term “polymorph” refers to a particular crystalline state of a substance, having particular physical properties such as X-ray diffraction, IR spectra, melting point, and the like.

### Phamaceutically Acceptable Salts

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention.

Compounds of Formula I, and salts, solvates thereof, may exist in their tautomeric form (for example, as an amide or imino ether). All such tautomeric forms are contemplated herein as part of the present invention.

### Pharmaceutical Compositions

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described potential method of treating a patient by administering a pharmaceutical composition of the present invention is also intended to encompass the administration of the afore-said bulk composition and individual dosage units.

Isolation of the compound at various stages of the reaction may be achieved by standard techniques such as, for example, filtration, evaporation of solvent and the like. Purification of the product and the like, may also be performed by standard techniques such as recrystallization, distillation, sublimation, chromatography, conversion to a suitable derivative. Such techniques are well known to those skilled in the art. The compounds of this invention may be analyzed for their composition and purity as well as characterized by standard analytical techniques such as, for example, elemental analysis, NMR, mass spectroscopy, and IR spectra.

In another embodiment, this invention provides pharmaceutical compositions comprising the compounds of the invention as an active ingredient. The pharmaceutical compositions generally additionally comprise a pharmaceutically acceptable carrier diluent, excipient or carrier (collectively referred to herein as carrier materials). The compounds can be administered orally or parenterally, including the intravenous, intramuscular, intraperitoneal, subcutaneous, rectal and/or topical routes of administration.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

For oral use of a compound according to this invention, particularly for chemotherapy, the selected compound may be administered, for example, in the form of tablets or capsules, or as an aqueous solution or suspension. In the case of tablets for oral use, carriers which are commonly used include lactose and cornstarch, and lubricating agents, such as magnesium stearate, are commonly added. For oral administration in capsule form, useful diluents include lactose and dried cornstarch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gels—refer to the active ingredients dispersed or solubilized in a hydrophillic semi-solid matrix.

Powders for constitution refer to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d,l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents—materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Additionally, the compositions of the present invention may be formulated in sustained release form to provide the rate controlled release of any one or more of the components or active ingredients to optimize the therapeutic effects, i.e. anti-cell proliferation activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a micro emulation.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

Another aspect of this invention is a kit comprising a therapeutically effective amount of at least one compound of Formula I, or a pharmaceutically acceptable salt or solvate of said compound and a pharmaceutically acceptable carrier, vehicle or diluent.

Yet another aspect of this invention is a kit comprising an amount of at least one compound of Formula I, or a pharmaceutically acceptable salt or solvate of said compound and an amount of at least one anticancer therapy and/or anti-cancer agent listed above, wherein the amounts of the two or more ingredients result in desired therapeutic effect.

### Utility

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

As previously mentioned, the Insulin-like Growth Factor-1 Receptor (IGF-1R) belongs to the family of transmembrane tyrosine kinase receptors such as platelet-derived growth factor receptor, the epidermal growth factor receptor, and the insulin receptor. There are two known ligands for the IGF-1R receptor. They are IGF-1 and IGF-2. As used herein, the term “IGF” refers to both IGF-1 and IGF-2. The insulin-like growth factor family of ligands, receptors and binding proteins is reviewed in Krywicki and Yee, Breast Cancer Research and Treatment, 22:7-19, 1992.

IGF/IGF-1R driven disorders are characterized by inappropriate or over-activity of IGF/IGF-1R. Inappropriate IGF activity refers to either: (1) IGF or IGF-1R expression in cells which normally do not express IGF or IGF-1R; (2) increased IGF or IGF-1R expression leading to unwanted cell proliferation such as cancer; (3) increased IGF or IGF-1R activity leading to unwanted cell proliferation, such as cancer; and/or over-activity of IGF or IGF-1R. Over-activity of IGF or IGF-1R refers to either an amplification of the gene encoding IGF-1, IGF-2, IGF-1R or the production of a level of IGF activity which can be correlated with a cell proliferative disorder (i.e., as the level of IGF increases the severity of one or more of the symptoms of the cell proliferative disorder increases) the bioavailability of IGF-1 and IGF-2 can also be affected by the presence or absence of a set of IGF binding presence or absence of a set of IGF binding proteins (IGF BPs) of which there are six known. Over activity of IGF/IGF-1R can also result from a down regulation of IGF-2 which contains an IGF-2 binding domain, but no intracellular kinase domain. Examples of IGF/IGF-1R driven disorders include the various IGF/IGF-1R related human malignancies reviewed in Cullen, et al., Cancer Investigation, 9(4):443-454, 1991, incorporated herein by reference in its entirety, including any drawings. IGF/IGF-1Rs clinical importance and role in regulating osteoblast function is reviewed in Schmid, Journal of Internal Medicine, 234:535-542, 1993.

Thus, IGF-1R activities include: (1) phosphorylation of IGF-1R protein; (2) phosphorylation of an IGF-1R protein substrate; (3) interaction with an IGF adapter protein; (4) IGF-1R protein surface expression. Additional IGF-1R protein activities can be identified using standard techniques. IGF-1R activity can be assayed by measuring one or more of the following activities: (1) phosphorylation of IGF-1R; (2) phosphorylation of an IGF-1R substrate; (3) activation of an IGF-1R adapter molecule; and (4) activation of downstream signaling molecules, and/or (5) increased cell division. These activities can be measured using techniques described below and known in the arts.

IGF-1R has been implicated as an absolute requirement for the establishment and maintenance of the transformed phenotype both in vitro and in vivo in several cell types (R. Baserga, Cancer Research 55:249-252, 1995). Herbimycin A has been said to inhibit the IGF-1R protein tyrosine kinase and cellular proliferation in human breast cancer cells (Sepp-Lorenzino, et al., 1994, J. Cell Biochem. Suppl. 18b: 246). Experiments studying the role of IGF-1R in transformation have used antisense strategies, dominant negative mutants, and antibodies to the IGF-1R and have led to the suggestion that IGR-1R may be a preferred target for therapeutic interventions.

IGF-1R, in addition to being implicated in nutritional support and in type-II diabetes, has also been associated with several types of cancers. For example, IGF-1 has been implicated as an autocrine growth stimulator for several tumor types, e.g. human breast cancer carcinoma cells (Arteago et al., J. Clin. Invest., 1989, 84:1418-1423) and small lung tumor cells (Macauley et al., Cancer Res., 1989, 50:2511-2517). In addition, IGF-1, while integrally involved in the normal growth and differentiation of the nervous system, also appears to be an autocrine stimulator of human gliomas. Sandberg-Nordqvist et al., Cancer Res., 1993, 53:2475-2478.

An example of IGF-2's potential involvement in colorectal cancer may be found in the up-regulation of IGF-2 mRNA in colon tumors relative to normal colon tissue. (Zhang et al., Science (1997) 276:1268-1272.) IGF-2 may also play a role in hypoxia induced neovascularization of tumors. (Minet et al., Int. J. Mol. Med. (2000) 5:253-259.) IGF-2 may also play a role in tumorigenesis through activation of an insulin receptor isoform-A. IGF-2 activation of insulin receptor isoform-A activates cell survival signaling pathways in cells but its relative contribution to tumor cell growth and survival is unknown at this time. Insulin receptor isoform-A's kinase domain is identical to the standard insulin receptor's. Scalia et al., 2001, J. Cell Biochem. 82:610-618.

The importance of IGF-1R and its ligands in cell types in culture (fibroblasts, epithelial cells, smooth muscle cells, T-lymphocytes, myeloid cells, chondrocytes and osteoblasts (the stem cells of the bone marrow)) is illustrated by the ability of IGF-1 to stimulate cell growth and proliferation. Goldring and Goldring, Eukaryotic Gene Expression, 1991, 1:301-326. In a series of recent publications, Baserga and others suggests that IGF-1R plays a central role in the mechanism of transformation and, as such, could be a preferred target for therapeutic interventions for a broad spectrum of human malignancies. Baserga, Cancer Res., 1995, 55:249-252; Baserga, Cell, 1994, 79:927-930; Coppola et al., Mol. Cell. Biol., 1994, 14:4588-4595; Baserga, Trends in Biotechnology, 1996, 14:150-152; H. M. Khandwala et al., Endocrine Reviews, 21:215-244, 2000. The predominant cancers that may be treated using a compound of the instant invention include, but are not limited to breast cancer, prostate cancer, colorectal cancer, small cell lung cancer, non-small cell lung cancer, renal cell carcinoma, or endometrial carcinoma.

The above referenced IGF-1R-related disorder may be a cancer selected from, but not limited to, astrocytoma, basal or squamous cell carcinoma, brain cancer, neuroblastoma, gliobastoma, liposarcoma, bladder cancer, breast cancer, colorectal cancer, colon cancer, gastric cancer, chrondrosarcoma, cervical cancer, adrenal cancer, choriocarcinoma, esophageal cancer, endometrial carcinoma, erythroleukemia, leukemia, multiple myeloma, Ewing's sarcoma, gastrointestinal cancer, head and neck cancer, hepatoma, glioma, hepatocellular carcinoma, leukemia, leiomyoma, melanoma, non-small cell lung cancer, neural cancer, ovarian cancer, pancreatic cancer, prostate cancer, renal cell carcinoma, rhabdomyosarcoma, small cell lung cancer, thyoma, thyroid cancer, testicular cancer and osteosarcoma in a further aspect of this invention. More preferably, the IGF-1R-related disorder is a cancer selected from brain cancer, breast cancer, prostate cancer, colorectal cancer, small cell lung cancer, non-small cell lung cancer, renal cell carcinoma or endometrial carcinoma.

Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

Included within the scope of the present invention is a pharmaceutical composition, which is comprised of a compound of Formula I as described above and a pharmaceutically acceptable carrier. The present invention also encompasses a potential method of treating or preventing cancer in a mammal in need of such treatment which is comprised of administering to said mammal a therapeutically effective amount of a compound of Formula I. Types of cancers which may be treated using compounds of Formula I include, but are not limited to, astrocytoma, basal or squamous cell carcinoma, brain cancer, gliobastoma, bladder cancer, breast cancer, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyona, melanoma, non-small cell lung cancer, neural cancer, ovarian cancer, pancreatic cancer, prostate cancer, renal cell carcinoma, rhabdomyosarcoma, small cell lung cancer, thymona, thyroid cancer, testicular cancer and osteosarcoma in a further aspect of this invention. More preferably, the cancer being treated is selected from breast cancer, prostate cancer, colorectal cancer, small cell lung cancer, non-small cell lung cancer, renal cell carcinoma, or endometrial carcinoma.

IGF-1 has also been associated with retinal neovascularization. Proliferative diabetes retinopathy has been seen in some patients having high levels of IGF-1. (L. E. Smith et al., Nature Medicine, 1999, 5:1390-1395.)

Compounds of the instant invention may also be useful as anti-aging agents. It has been observed that there is a link between IGF signalling and aging. Experiments have shown that calorie-restricted mammals have low levels of insulin and IGF-1 and have a longer life span. Similar observations have been made for insects as well. (See C. Kenyon, Cell, 2001, 105:165-168; E. Strauss, Science, 2001, 292:41-43; K. D. Kimura et al., Science 1997, 277:942-946; M. Tatar et al., Science, 2001, 292:107-110).

IGFR-related disorder also include but are not limited to diabetes, an autoimmune disorder, Alzheimer's and other cognitive disorders, a hyperproliferation disorder, aging, cancer, acromegaly, Crohn's disease, endometriosis, diabetic retinopathy, restenosis, fibrosis, psoriasis, osteoarthritis, rheumatoid arthritis, an inflammatory disorder and angiogenesis in yet another aspect of this invention.

A potential method of treating or preventing retinal vascularization which is comprised of administering to a mammal in need of such treatment a therapeutically effective amount of compound of Formula I is also encompassed by the present invention. Potential methods of treating or preventing ocular diseases, such as diabetic retinopathy and age-related macular degeneration, are also part of the invention.

Also included within the scope of the present invention is a potential method of treating or preventing inflammatory diseases, such as rheumatoid arthritis, psoriasis, contact dermatitis and delayed hypersensitivity reactions, as well as treatment or prevention of bone associated pathologies selected from osteosarcoma, osteoarthritis, and rickets.

Other disorders which might be treated with compounds of this invention include, without limitation, immunological and cardiovascular disorders such as atherosclerosis.

The invention also contemplates the use of the instantly claimed compounds in combination with a second compound selected from the group consisting of:

1) an estrogen receptor modulator,

2) an androgen receptor modulator,

3) retinoid receptor modulator,

4) a cytotoxic agent,

5) an antiproliferative agent,

6) a prenyl-protein transferase inhibitor,

7) an HMG-CoA reductase inhibitor,

8) an HIV protease inhibitor,

9) a reverse transcriptase inhibitor, and

10) angiogenesis inhibitor.

A preferred angiogenesis inhibitor is selected from the group consisting of a tyrosine kinase inhibitor, an inhibitor of epidermal-derived growth factor, an inhibitor of fibroblast-derived growth factor, an inhibitor of platelet derived growth factor, an MMP inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGF. Preferred estrogen receptor modulators are tamoxifen and raloxifene.

Also included in the scope of the claims is a potential method of treating cancer, which comprises administering a therapeutically effective amount of a compound of Formula I in combination with a compound selected from the group consisting of:

1) an estrogen receptor modulator,

2) an androgen receptor modulator,

3) retinoid receptor modulator,

4) a cytotoxic agent,

5) an antiproliferative agent,

6) a prenyl-protein transferase inhibitor,

7) an HMG-CoA reductase inhibitor,

8) an HIV protease inhibitor,

9) a reverse transcriptase inhibitor, and

10) angiogenesis inhibitor.

And yet another embodiment is the potential method of treating cancer using the combination discussed above, in combination with radiation therapy.

And yet another embodiment of the invention is a potential method of treating cancer which comprises administering a therapeutically effective amount of a compound of Formula I in combination with paclitaxel or trastuzumab.

### Combination Therapy

The instant compounds are also useful in combination with therapeutic, chemotherapeutic and anti-cancer agents. Combinations of the presently disclosed compounds with therapeutic, chemotherapeutic and anti-cancer agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. The instant compounds may be useful when co-administered with radiation therapy. The compounds of the present invention can be present in the same dosage unit as the anticancer agent or in separate dosage units.

Another aspect of the present invention is a potential method of treating one or more diseases associated with IGF-1R or IR, comprising administering to a mammal in need of such treatment an amount of a first compound, which is a compound of the present invention, or a pharmaceutically acceptable salt or solvate thereof; and an amount of at least one second compound, the second compound being an anti-cancer agent different from the compounds of the present invention, wherein the amounts of the first compound and the second compound result in a potential therapeutic effect.

Non-limiting examples of suitable anti-cancer agents include cytostatic agents, cytotoxic agents, targeted therapeutic agents (small molecules, biologics, siRNA and microRNA) against cancer and neoplastic diseases,
1) anti-metabolites (such as methoxtrexate, 5-fluorouracil, gemcitabine, fludarabine, capecitabine);
2) alkylating agents, such as temozolomide, cyclophosphamide,
3) DNA interactive and DNA damaging agents, such as cisplatin, oxaliplatin, doxorubicin,
4) Ionizing irradiation, such as radiation therapy,
5) topoisomerase II inhibitors, such as etoposide, doxorubicin,
6) topoisomerase I inhibitors, such as irinotecan, topotecan,
7) tubulin interacting agents, such as paclitaxel, docetaxel, Abraxane, epothilones,
8) kinesin spindle protein inhibitors,
9) spindle checkpoint inhibitors,
10) Poly(ADP-ribose) polymerase (PARP) inhibitors, such as olaparib, MK-4827 and veliparib
11) Matrix metalloprotease (MMP) inhibitors
12) Protease inhibitors, such as cathepsin D and cathepsin K inhibitors
13) Proteosome or ubiquitination inhibitors, such as bortezomib,
14) Activator of mutant p53 to restore its wild-type p53 activity
15) Adenoviral-p53
16) Bcl-2 inhibitors, such as ABT-263
17) Heat shock protein (HSP) modulators, such as geldanamycin and 17-AAG
18) Histone deacetylase (HDAC) inhibitors, such as vorinostat (SAHA),
19) sex hormone modulating agents,
   a. anti-estrogens, such as tamoxifen, fulvestrant,
   b. selective estrogen receptor modulators (SERM), such as raloxifene,
   c. anti-androgens, such as bicalutamide, flutamide
   d. LHRH agonists, such as leuprolide,
   e. 5α-reductase inhibitors, such as finasteride,
   f. Cytochrome P450 C₁₇ lyase (CYP450c17, also called 17α-hydroxylase/17,20 lysase) inhibitors, such as Abiraterone acetate, VN/124-1, TAK-700
   g. aromatase inhibitors, such as letrozole, anastrozole, exemestane,
20) EGFR kinase inhibitors, such as geftinib, erlotinib, laptinib
21) dual erbB1 and erbB2 inhibitors, such as Lapatinib
22) multi-targeted kinases (serine/threonine and/or tyrosine kinase) inhibitors,
   a. ABL kinase inhibitors, imatinib and nilotinib, dasatinib
   b. VEGFR-1, VEGFR-2, PDGFR, KDR, FLT, c-Kit, Tie2, Raf, MEK and ERK inhibitors, such as sunitinib, sorafenib, Vandetanib, pazopanib, PLX-4032, Axitinib, PTK787, GSK-1120212
   c. Polo-like kinase inhibitors,
   d. Aurora kinase inhibitors,
   e. JAK inhibitor
   f. c-MET kinase inhibitors
   g. Cyclin-dependent kinase inhibitors, such as CDK1 and CDK2 inhibitor SCH 727965
   h. PI3K and mTOR inhibitors, such as GDC-0941, BEZ-235, BKM-120 and AZD-8055
   i. Rapamycin and its analogs, such as Temsirolimus, everolimus, and deforolimus
23) and other anti-cancer (also know as anti-neoplastic) agents include but are not limited to ara-C, adriamycin, cytoxan, Carboplatin, Uracil mustard, Clormethine, Ifosfsmide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Vinorelbine, Navelbine, Bleomycin, Dactinomycin, Daunorubicin, Doxorubicin, Epirubicin, teniposide, cytarabine, pemetrexed, Idarubicin, Mithramycin, Deoxycoformycin, Mitomycin-C, L-Asparaginase, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Flutamide Medroxyprogesteroneacetate, Toremifene, goserelin, Carboplatin, Hydroxyurea, Amsacrine, Procarbazine, Mitotane, Mitoxantrone, Levamisole, Drolloxafine, Hexamethylmelamine, Bexxar, Zevalin, Trisenox, Profimer, Thiotepa, Altretamine, Doxil, Ontak, Depocyt, Aranesp, Neupogen, Neulasta, Kepivance.
24) Farnesyl protein transferase inhibitors, such as, SARASAR™(4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-piperidinecarboxamide, tipifarnib
25) interferons, such as Intron A, Peg-Intron,
26) anti-erbB1 antibodies, such as cetuximab, panitumumab,
27) anti-erbB2 antibodies, such as trastuzumab,
28) anti-CD52 antibodies, such as Alemtuzumab,
29) anti-CD20 antibodies, such as Rituximab
30) anti-CD33 antibodies, such as Gemtuzumab ozogamicin
31) anti-VEGF antibodies, such as Avastin,
32) TRIAL ligands, such as Lexatumumab, mapatumumab, and AMG-655
33) Anti-CTLA-4 antibodies, such as ipilimumab
34) antibodies against CTAT, CEA, CD5, CD19, CD22, CD30, CD44, CD44V6, CD55, CD56, EpCAM, FAP, MHCII, HGF, IL-6, MUC₁, PSMA, TALE, TAG-72, TRAILR, VEGFR, IGF-2, FGF,
35) anti-IGF-1R antibodies, such as dalotuzumab (MK-0646) and robatumumab (SCH 717454)

If formulated as a fixed dose such combination products employ the compounds of this invention within the dosage range described herein and the other pharmaceutically active agent or treatment within its dosage range. Compounds of Formula I may also be administered sequentially with known anticancer or cytotoxic agents when a combination formulation is inappropriate. The invention is not limited in the sequence of administration; compounds of Formula I may be administered either concurrent with, prior to or after administration of the known anticancer or cytotoxic agent. Such techniques are within the skills of the persons skilled in the art as well as attending physicians.

Accordingly, in an aspect, this invention includes combinations comprising an amount of at least one compound of Formula I, or a pharmaceutically acceptable salt or solvate thereof, and an amount of one or more anti-cancer treatments and anti-cancer agents listed above wherein the amounts of the compounds/treatments result in potential therapeutic effect.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, platinum coordinator compounds, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinumMbis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, 4-demethoxy-3-deamino-3-aziridinyl-4-methylsulphonyl-daunorubicin (see WO 00/50032).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include taxanes in general. Specific compounds include paclitaxel (Taxol®), vindesine sulfate, 3′,4′-didehydro-4′-deoxy-8′-norvincaleukoblastine, docetaxol (Taxotere®), rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the CHK11 and CHK12 kinases and cdk and cdc kinase inhibitors and are specifically exemplified by 7-hydroxystaurosporin, flavopiridol, CYC₂₀₂ (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734,188, 60/652,737, 60/670,469), inhibitors of Raf kinase (for example PLX-4032), inhibitors of MEK (for example Arry-162, RO-4987655 and GSK-1120212), inhibitors of mTOR (for example AZD-8055, BEZ-235 and everolimus), and inhibitors of PI3K (for example GDC-0941, BKM-120).

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272 and U.S. Pat. No. 5,932,598.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to, the following: parecoxib, BEXTRA® and CELEBREX® or a pharmaceutically acceptable salt thereof.

Other examples of angiogenesis inhibitors include, but are not limited to, endostatin, ukrain, ranpirnase, IM862,5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382,2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists may be useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the potential use of the presently disclosed compounds in combination with gene therapy for the treatment of cancer. For an overview of genetic strategies to treating cancer see Hall et al (Am. J. Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC₁₄₄-093, R101922, VX853 and PSC₈₃₃ (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir.

A compound of the instant invention may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, Cl1033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC₁₄₄-093, erythromycin, vincristine, digoxin and talinolol.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane.

A compound of the instant invention may also be useful for treating or preventing cancer in combination with siRNA therapeutics.

The compounds of the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

Inhibitors of Akt, as disclosed in the following publications; WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734,188, 60/652,737, 60/670,469, and including compounds of the instant invention, are also useful in combination with potassium salts, magnesium salts, beta-blockers (such as atenolol) and endothelin-a (ETa)antagonists with the goal of maintaining cardiovascular homeostasis.

Inhibitors of Akt, as disclosed in the following publications; WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734,188, 60/652,737, 60/670,469, and including compounds of the instant invention, are also useful in combination with insulin, insulin secretagogues, PPAR-gamma agonists, metformin, somatostatin receptor agonists such as octreotide, DPP4 inhibitors, sulfonylureas and alpha-glucosidase inhibitors with the goal of maintaining glucose homeostasis.

A compound of the instant invention may also be useful for treating or preventing cancer in combination with PARP inhibitors: olaparib, MK-4827 and veliparib.

A compound of the instant invention may also be useful for treating cancer in combination with the following chemotherapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxane®); bortezomib (Velcade®); brefeldin A; busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); dalteparin sodium injection (Fragmin®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); didemnin B; 17-DMAG; docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); ethinyl estradiol; etoposide phosphate (Etopophos®); etoposide, VP-16 (Vepesid®); everolimus tablets (Afinitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); geldanamycin; gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); iobenguane 1123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); 8-methoxypsoralen; mitomycin C (Mutamycin®); mitotane (Lysodren®); mitoxantrone (Novantrone®); mitramycin; nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); rapamycin; Rasburicase (Elitek®); raloxifene hydrochloride (Evista®); Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiopurine; thiotepa (Thioplex®); topotecan (Hycamtin®); toremifene (Fareston®); Tositumomab (Bexxar®); Tositumomab/I-131 tositumomab (Bexxar®); trans-retinoic acid; Trastuzumab (Herceptin®); tretinoin, ATRA (Vesanoid®); triethylenemelamine; Uracil Mustard (Uracil Mustard Capsules®); valrubicin (Valstar®); vinblastine (Velban®); vincristine (Oncovin®); vinorelbine (Navelbine®); vorinostat (Zolinza®); wortmannin; and zoledronate (Zometa®).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA), the Physician's Desk Reference, 56^{th} Edition, 2002 (published by Medical Economics company, Inc. Montvale, N.J. 07645-1742), and the Physician's Desk Reference, 57^{th} Edition, 2003 (published by Thompson PDR, Montvale, N.J. 07645-1742).

The compounds of this invention may be prepared by employing reactions as shown in the following schemes, in addition to other standard manipulations that are known in the literature or exemplified in the experimental procedures. These schemes, therefore, are not limited by the compounds listed nor by any particular substituents employed for illustrative purposes. Substituent numbering, as shown in the schemes, does not necessarily correlate to that used in the claims.

### Example 1

### Synthesis

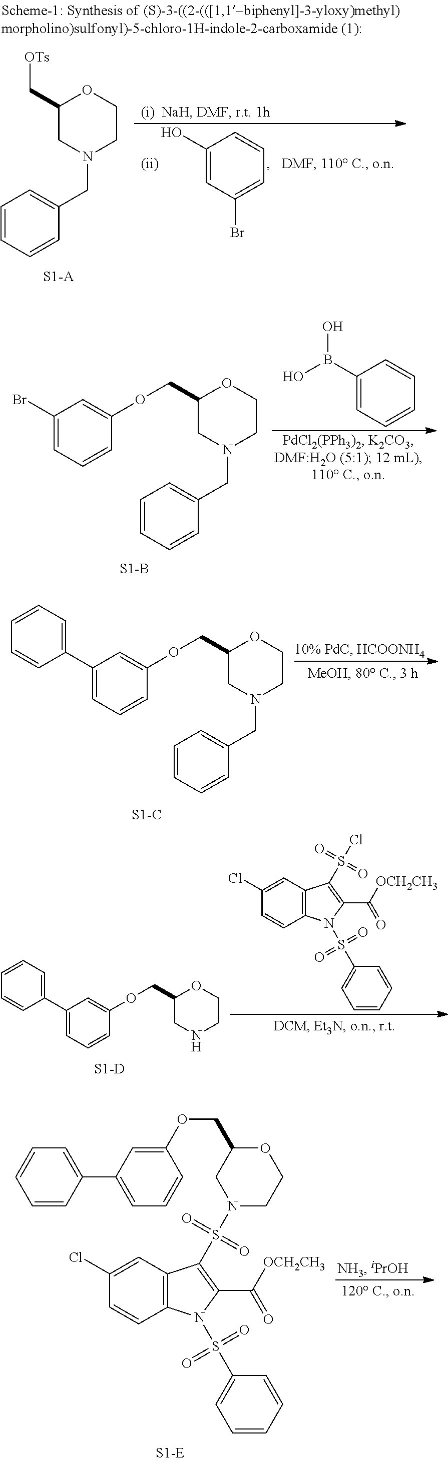

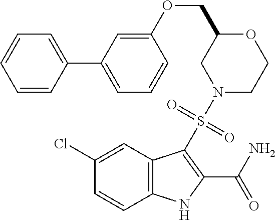 [SMILES: NC(=O)c1[nH]c2ccc(Cl)cc2c1S(=O)(=O)N1CCO[C@H](COc2cccc(-c3ccccc3)c2)C1]

### Step-1. Preparation of (S)-4-Benzyl-2-((3-bromophenoxy)methyl)morpholine (1-B)

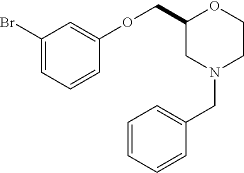

A solution of m-bromophenol (1 eq) in DMF was added to a mixture of NaH (1 eq) in DMF at room temperature. The reaction mixture was initially stirred at ambient temperature for 10 minutes. The sodium alkoxide solution was then charged with (S)-(4-benzylmorpholin-2-yl)methyl 4-methylbenzenesulfonate (1 eq) in DMF and the resulting reaction mixture was heated at 110° C. for 2 h. The solution was concentrated under reduced pressure; residue obtained was diluted with ethyl acetate (50 mL), washed with water (10 mL) and brine (5 mL). The combined organic layers were dried over anhydrous Na₂SO₄, filtered, concentrated in vacuo, and the residue obtained was purified by flash column chromatography (230-400 mesh size silica gel, 40% EtOAc/pet. ether gradient elution) to yield (S)-4-benzyl-2-((3-bromophenoxy)methyl)morpholine as a gummy oil.

¹H NMR (CDCl₃, 300 MHz): δ 7.36-7.07 (m, 8H), 6.87-6.83 (m, 1H), 4.00-3.88 (m, 4H), 3.81 (dt, J=11.4 and 2.4 Hz, 1H), 3.56 (s, 2H), 2.89 (d, J=11.4 Hz, 1H), 2.74 (d, J=11.1 Hz, 1H), 2.30 (dt, J=11.4 and 3.3 Hz, 1H), 2.15 (t, J=11.4 Hz, 1H).

MS (m/z): 362 (M⁺+1) and 364 (M⁺+1)

### Step-2. Preparation of (S)-2-(([1,1′-biphenyl]-3-yloxy)methyl)-4-benzylmorpholine (S1-C)

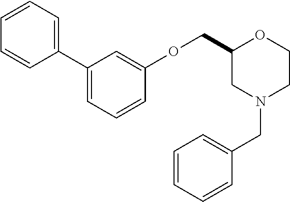

A mixture of (S)-4-benzyl-2-((3-bromophenoxy)methyl)morpholine (1 eq), phenylboronic acid (1.2 eq) and potassium carbonate (1.4 eq) in DMF:H₂O (2:1) (12 mL) were sonicated for two minutes and stirred under argon atmosphere at an ambient temperature. The dichlorobis(triphenylphosphine)Palladium(II) (0.1 eq) was added to the reaction mixture and stirred at 120° C. until complete consumption of the starting materials was indicated by TLC analysis (˜3 h). The reaction was diluted with EtOAc (50 mL) and filtered through a celite bed. The filtrate was washed with water (20 mL) and brine (5 mL). The organic layer was dried over anhydrous Na₂SO₄, filtered, concentrated in vacuo and the residue obtained was purified by flash column chromatography (200-400 mesh size silica gel, 30% ethyl acetate/pet. ether, gradient elution) to yield (S)-2-(([1,1′-biphenyl]-3-yloxy)methyl)-4-benzylmorpholine as a gummy oil.

¹H NMR (CDCl₃, 300 MHz): δ 7.60 (d, J=1.5 Hz, 2H), 7.57 (m, 2H), 7.44-7.30 (m, 6H), 7.16-6.92 (m, 2H), 6.92-6.88 (m, 2H), 4.10-3.94 (m, 4H), 3.82 (m, 1H), 3.60 (s, 2H), 2.97 (m, 1H), 2.77 (d, J=10.8 Hz, 1H), 2.30-2.19 (m, 2H).

MS (m/z): 360 (M⁺)

### Step-3. Preparation of (S)-2-(([1,1′-biphenyl]-3-yloxy)methyl)morpholine (S1-D)

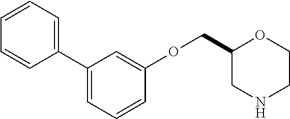

To a methanolic solution (20 mL) of (S)-2-(([1,1′-biphenyl]-3-yloxy)methyl)-4-benzylmorpholine (1 eq) and ammonium formate (5 eq) in a two-necked round bottom flask Pd/C was added under argon atmosphere. The reaction mixture was refluxed till complete consumption of starting material as indicated by TLC (3 h). The reaction was filtered through celite bed and filtrate obtained was concentrated in vacuo and the residue obtained was purified by flash column chromatography (200-400 mesh size silica gel, 10% methanol/dichloromethane, gradient elution) to yield (S)-2-(([1,1′-biphenyl]-3-yloxy)methyl)morpholine as a gummy oil.

¹H NMR (CDCl₃, 300 MHz): δ 7.60 (d, J=2.1 Hz, 2H), 7.58-7.46 (m, 2H), 7.36 (m, 2H), 7.22 (m, 2H), 6.93 (m, 1H), 4.10 (m, 1H), 4.06-3.92 (m, 3H), 3.79-3.74 (m, 1H), 3.15 (d, J=13 Hz, 1H), 2.99-2.80 (m, 3H).

MS (m/z): 269.7 (M⁺+1), 241.9, 255.9

### Step-4. Preparation of (S)-ethyl 3-((2-(([1,1′-biphenyl]-3-yloxy)methyl)morpholino)sulfonyl)-5-chloro-1-(phenylsulfonyl)-1H-indole-2-carboxylate (S1-E)

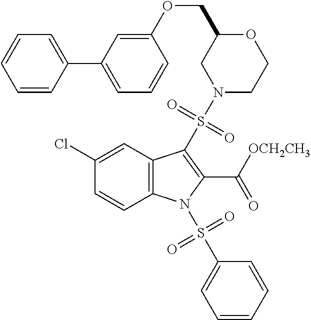

To a solution of (S)-2-(([1,1′-biphenyl]-3-yloxy)methyl)morpholine (1 eq) in dichloromethane (10 mL), triethyl amine (5 eq) was added and the reaction mixture was stirred under argon atmosphere. Ethyl 5-chloro-3-(chlorosulfonyl)-1-(phenylsulfonyl)-1H-indole-2-carboxylate (1.2 eq) was added to reaction mixture and the resulting reaction mixture was stirred for approximately 16 h at ambient temperature. The reaction was concentrated in vacuo, crude material was adsorbed on silica gel and subjected to column chromatography (200-400 mesh size) using 5% methanol/dichloromethane as gradient eluent to furnish (S)-ethyl 3-((2-(([1,1′-biphenyl]-3-yloxy)methyl)morpholino)sulfonyl)-5-chloro-1-(phenylsulfonyl)-1H-indole-2-carboxylate as a yellowish solid.

¹H NMR (CDCl₃, 300 MHz): δ 8.08 (m, 2H), 8.08 (d, J=23.7 Hz, 1H), 7.89 (s, 1H), 7.63-7.15 (m, 11H), 7.15 (d, J=2.1 Hz, 1H), 7.14 (s, 1H), 6.91 (d, J=2.1 Hz, 1H), 4.61 (m, J=6.9, 7.2 and 7.2 Hz, 2H), 4.10 (m, 1H), 4.02-3.92 (m, 4H), 3.76-3.72 (m, 2H), 2.88-2.79 (m, 1H), 2.75-2.69 (m, 1H), 1.49-1.44 (t, J=7.2 Hz, 3H)

MS (m/z): 695.2 (M⁺)

### Step-5. Preparation of (S)-3-((2-(([1,1′-biphenyl]-3-yloxy)methyl)morpholino)sulfonyl)-5-chloro-1H-indole-2-carboxamide (1)

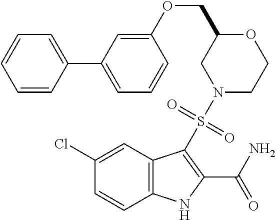

To a solution of (S)-ethyl 3-((2-(([1,1′-biphenyl]-3-yloxy)methyl)morpholino)sulfonyl)-5-chloro-1-(phenylsulfonyl)-1H-indole-2-carboxylate (1 eq) in isopropyl alcohol (20 mL) at 0° C. ammonia gas was bubbled for 15 minutes. The resulting solution was sealed in seal-tube and placed in an oil bath at 120° C. for 16 h. The solvent was removed under reduced pressure. Crude residue was subjected to flash column chromatography (200-400 mesh size silica gel, 10% methanol/dichloromethane, gradient elution) to afford amide (S)-3-((2-(([1,1′-biphenyl]-3-yloxy)methyl)morpholino)sulfonyl)-5-chloro-1H-indole-2-carboxamide (1) as a yellowish solid.

¹H NMR (CDCl₃, 300 MHz): δ 10.8 (s, 1H), 9.09 (s, 1H), 8.16 (s, 1H), 7.97 (d, J=7.2 Hz, 2H), 7.63-7.31 (m, 14H), 7.22 (d, J=7.5 Hz, 1H), 7.09 (s, 1H), 6.86 (m, J=5.7, 2.4 and 2.1 Hz, 1H), 6.13 (s, 1H), 4.09-3.94 (m, 4H), 3.83-3.76 (m, 2H), 3.64 (d, J=11.4 Hz, 1H), 2.64 (m, J=7.8 and 3.6 Hz, 1H), 2.64-2.46 (m, J=10.8 and 10.2 Hz, 1H).

MS (m/z): 524 [M−H]

### Preparation of (S)-5-chloro-3-((2-((3-(pyridin-4-yl)phenoxy)methyl)morpholino)sulfonyl)-1H-indole-2-carboxamide (2)

Synthesized by similar route as represented in scheme-1 by using pyridine-4-boronic acid in step-2.

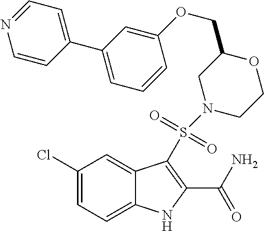 [SMILES: NC(=O)c1[nH]c2ccc(Cl)cc2c1S(=O)(=O)N1CCO[C@H](COc2cccc(-c3ccncc3)c2)C1]

¹H NMR (DMSO-d₆, 300 MHz): δ 13.04 (s, 1H), 8.63 (s, 1H), 8.61 (s, 1H), 8.261 (d, J=12.6 Hz, 2H), 7.91 (s, 1H), 7.71 (d, J=6 Hz, 2H), 7.59 (d, J=8.7 Hz, 1H), 7.34-7.30 (m, 4H), 7.02 (s, 1H), 5.76 (s, 1H), 4.09 (d, J=4.5 Hz, 2H), 3.9 (m, 2H), 3.7 (m, 1H), 3.57 (m, 2H), 2.50 (m, 2H).

MS (m/z): 527 [M+H]

### Preparation of (S)-5-chloro-3-((2-((3-(2-fluoropyridin-3-yl)phenoxy)methyl)morpholino)sulfonyl)-1H-indole-2-carboxamide (3)

Synthesized by similar route as represented in scheme-1 by using 2-fluoropyridine-3-boronic acid in step-2.

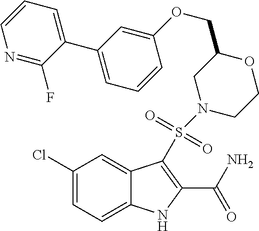 [SMILES: NC(=O)c1[nH]c2ccc(Cl)cc2c1S(=O)(=O)N1CCO[C@H](COc2cccc(-c3cccnc3F)c2)C1]

¹H NMR (CDCl₃, 300 MHz): δ 9.07 (s, 1H), 8.22-821 (d, J=4.8 Hz, 1H), 8.15 (s, 1H), 7.97-7.94 (d, J=6.9 Hz, 1H), 7.87-7.84 (m, 1H), 7.61-7.59 (d, J=7.2 Hz, 1H), 7.54-7.48 (m, 1H), 7.40-7.31 (m, 1H), 7.19-7.16 (d, J=7.2 Hz, 1H), 7.07 (s, 1H), 6.93-6.91 (m, J=2.4 Hz, 1H), 4.08-4.04 (m, 1H), 3.99-3.93 (m, 3H), 3.82-3.73 (m, 2H), 3.67-3.61 (d, J=10.8 Hz, 1H), 2.64-2.60 (m, 1H), 2.52-2.45 (m, 1H).

MS (m/z): 545 [M+H]

### Preparation of (S)-5-chloro-3-((2-((3-(6-methylpyridin-3-yl)phenoxy)methyl)morpholino)sulfonyl)-1H-indole-2-carboxamide (4)

Synthesized by similar route as represented in scheme-1 by using 2-methylpyridine-5-boronic acid in step-2.

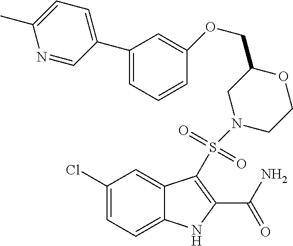 [SMILES: Cc1ccc(-c2cccc(OC[C@@H]3CN(S(=O)(=O)c4c(C(N)=O)[nH]c5ccc(Cl)cc45)CCO3)c2)cn1]

¹H NMR (CDCl₃, 300 MHz): δ 10.52 (br. s, 1H), 9.04 (br. s, 1H), 8.69 (d, J=1.8 Hz, 1H), 8.14 (d, J=1.8 Hz, 1H), 7.76 (dd, J=8.1 and 2.4 Hz, 1H), 7.48 (d, J=8.7 Hz, 1H), 7.37-7.32 (m, 2H), 7.22 (d, J=8.1 Hz, 1H), 7.17 (d, J=8.1 Hz, 1H), 7.02 (s, 1H), 6.87 (dd, J=8.1 and 2.1 Hz, 1H), 6.12 (br. s, 1H), 4.06-3.95 (m, 4H), 3.79-3.71 (m, 2H), 3.62 (d, J=11.1 Hz, 1H), 2.59 (s, 3H), 2.59-2.44 (m, 2H).

MS: (m/z) 541 [M+H], 149, 102

### Preparation of (S)-5-chloro-3-((2-((3-(6-(dimethylamino)pyridin-3-yl)phenoxy)methyl)morpholino)sulfonyl)-1H-indole-2-carboxamide (5)

Synthesized by similar route as represented in scheme-1 by using 2-N,N-dimethyl-5-boronic acid in step-2.

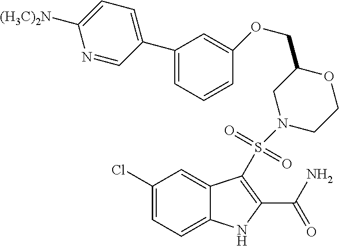

¹H NMR (CDCl₃, 300 MHz): δ 10.49 (br. s, 1H), 9.07 (br. s, 1H), 8.42 (d, J=2.4 Hz, 1H), 8.16 (d, J=1.5 Hz, 1H), 7.69 (dd, J=8.7 and 2.4 Hz, 1H), 7.50 (d, J=9.0 Hz, 1H), 7.39-7.31 (m, 2H), 7.15 (d, J=7.8 Hz, 1H), 7.00 (s, 1H), 6.80 (dd, J=8.1 and 2.1 Hz, 1H), 6.60 (d, J=8.7 Hz, 1H), 6.13 (br. s, 1H), 4.07-3.93 (m, 4H), 3.82-3.73 (m, 2H), 3.64 (d, J=11.7 Hz, 1H), 3.14 (s, 6H), 2.64 (dt, J=11.4 and 3.3 Hz, 1H), 2.52 (t, J=10.2 Hz, 1H).

MS: (m/z) 570 [M+H]

### Synthesis of (S)-5-chloro-3-((2-((3-(6-(trifluoromethyl)pyridin-3-yl)phenoxy)methyl)morpholino)sulfonyl)-1H-indole-2-carboxamide (6)

Synthesized by similar route as represented in scheme-1 by using 2-trifluoromethyl-5-boronic acid in step-2.

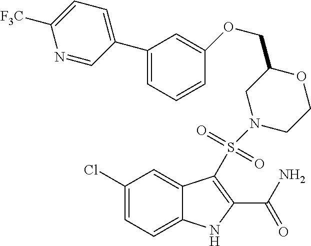 [SMILES: NC(=O)c1[nH]c2ccc(Cl)cc2c1S(=O)(=O)N1CCO[C@H](COc2cccc(-c3ccc(C(F)(F)F)nc3)c2)C1]

¹H NMR (CDCl₃, 300 MHz): δ 10.40 (br. s, 1H), 8.16 (br. s, 1H), 8.04 (dd, J=6.6 and 1.8 Hz, 1H), 7.78 (d, J=8.1 Hz, 1H), 7.50-7.36 (m, 5H), 7.22 (d, J=7.8 Hz, 1H), 7.09 (s, 1H), 6.98 (dd, J=8.4 and 2.1 Hz, 1H), 6.07 (br. s, 1H), 4.09-3.98 (m, 4H), 3.82-3.74 (m, 2H), 3.65 (d, J=12.0 Hz, 1H), 2.61 (dt, J=11.4 and 3.0 Hz, 1H), 2.50 (t, J=9.9 Hz, 1H).

MS: (m/z) 595 [M+H]

### Preparation of (S)-5-chloro-3-((2-((3-(6-methoxypyridin-3-yl)phenoxy)methyl)morpholino)sulfonyl)-1H-indole-2-carboxamide (7)

Synthesized by similar route as represented in scheme-1 by using 2-methoxypyridine-5-boronic acid in step-2.

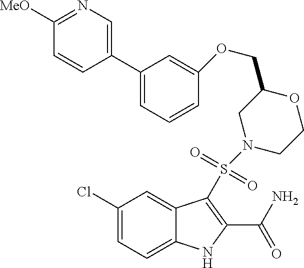 [SMILES: COc1ccc(-c2cccc(OC[C@@H]3CN(S(=O)(=O)c4c(C(N)=O)[nH]c5ccc(Cl)cc45)CCO3)c2)cn1]

¹H NMR (CDCl₃, 300 MHz): δ 10.20 (br. s, 1H), 9.06 (br. s, 1H), 8.37 (d, J=2.1 Hz, 1H), 8.16 (d, J=1.8 Hz, 1H), 7.79 (dd, J=8.4 and 2.4 Hz, 1H), 7.50 (d, J=8.7 Hz, 1H), 7.40 (dd, J=8.7 and 1.8 Hz, 1H), 7.35 (d, J=7.8 Hz, 1H), 7.15 (d, J=7.8 Hz, 1H), 7.02 (s, 1H), 6.87-6.81 (m, 2H), 6.12 (br. s, 1H), 4.08-3.94 (m, 7H), 3.81-3.74 (m, 2H), 3.64 (d, J=12.0 Hz, 1H), 2.65 (dt, J=11.4 and 3.0 Hz, 1H), 2.45 (t, J=9.9 Hz, 1H).

MS: (m/z) 557 [M+H]

### Preparation of (S)-5-chloro-3-((2-((3-(6-fluoropyridin-3-yl)phenoxy)methyl)morpholino)sulfonyl)-1H-indole-2-carboxamide (8)

Synthesized by similar route as represented in scheme-1 by using 2-fluoropyridine-5-boronic acid in step-2.

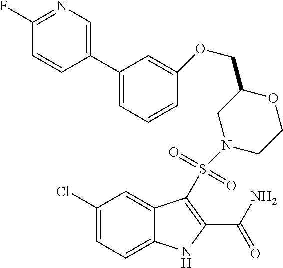 [SMILES: NC(=O)c1[nH]c2ccc(Cl)cc2c1S(=O)(=O)N1CCO[C@H](COc2cccc(-c3ccc(F)nc3)c2)C1]

¹H NMR (CDCl₃, 300 MHz): δ 10.20 (br. s, 1H), 9.05 (br. s, 1H), 8.41 (d, J=2.1 Hz, 1H), 8.16 (d, J=1.8 Hz, 1H), 7.99 (dd, J=8.1 and 2.4 Hz, 1H), 7.50 (d, J=8.7 Hz, 1H), 7.41-7.36 (m, 2H), 7.16 (d, J=7.5 Hz, 1H), 7.02-7.00 (m, 2H), 6.92 (dd, J=8.1 and 2.4 Hz, 1H), 6.06 (br. s, 1H), 4.08-3.95 (m, 4H), 3.82-3.73 (m, 2H), 3.62 (d, J=12.0 Hz, 1H), 2.65 (dt, J=11.4 and 3.3 Hz, 1H), 2.45 (t, J=9.9 Hz, 1H).

MS: (m/z) 545 [M+H]

### Preparation of (S)-5-chloro-3-((2-((3′-(dimethylamino)-[1,1′-biphenyl]-3-yl)oxy)methyl)morpholino)sulfonyl)-1H-indole-2-carboxamide (9)

Synthesized by similar route as represented in scheme-1 by using (3-(dimethylamino)phenyl)boronic acid in step-2.

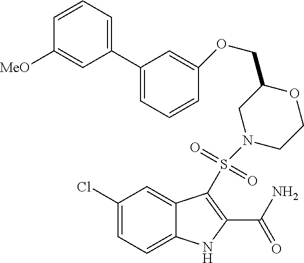 [SMILES: COc1cccc(-c2cccc(OC[C@@H]3CN(S(=O)(=O)c4c(C(N)=O)[nH]c5ccc(Cl)cc45)CCO3)c2)c1]

¹H NMR (CDCl₃, 300 MHz): δ 10.61 (s, 1H), 9.12 (s, 1H), 8.16 (dJ=1.5 Hz, 1H), 7.48 (d, J=8.7 Hz, 1H), 7.39 (d, J=1.8 Hz, 1H), 7.27-7.35 (m, 2H), 7.22 (d, J=7.8 Hz, 1H), 7.10 (br. s, 1H), 6.83 (m, J=8.1, 1H), 6.79 (d, J=7.5, 1H), 6.50 (m, 2H), 6.10 (br. s, 1H), 4.05-3.60 (m, 4H), 3.83-3.73 (m, 2H), 3.60 (d, J=11.6, 1H), 3.02 (s, 6H), 2.59 (m, 1H), 2.49 (t, J=10.8 Hz, 1H).

MS: (m/z) 569 [M+H]

### Preparation of (S)-5-chloro-3-((2-(((2′-methyl-[1,1′-biphenyl]-3-yl)oxy)methyl)morpholino)sulfonyl)-1H-indole-2-carboxamide (10)

Synthesized by similar route as represented in scheme-1 by using o-tolylboronic acid in step-2.

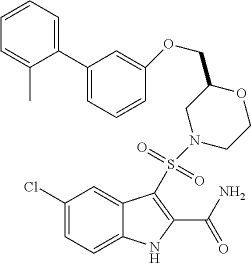 [SMILES: Cc1ccccc1-c1cccc(OC[C@@H]2CN(S(=O)(=O)c3c(C(N)=O)[nH]c4ccc(Cl)cc34)CCO2)c1]

¹H NMR (DMSO-d₆, 300 MHz): δ 13.04 (s, 1H), 8.25 (d, J=15 Hz, 2H), 7.9 (d, J=1.5 Hz, 1H), 7.50 (d, J=9 Hz, 1H), 7.35-7.33 (m, 2H), 7.31-7.18 (m, 4H), 6.89 (d, J=9 Hz, 2H), 6.81 (s, 1H), 4.01 (d, J=3 Hz, 2H), 4.03-4.01 (m, 1H), 3.80-3.78 (m, 1H), 3.69-3.60 (m, 1H), 2.50-2.40 (m, 2H), 2.21-2.20 (m, 2H), 2.2 (s, 3H).

MS: (m/z) 540 [M+H]

### Preparation of (S)-5-chloro-3-((2-(((4′-fluoro-[1,1′-biphenyl]-3-yl)oxy)methyl)morpholino)sulfonyl)-1H-indole-2-carboxamide (11)

Synthesized by similar route as represented in scheme-1 by using (4-fluorophenyl)boronic acid in step-2.

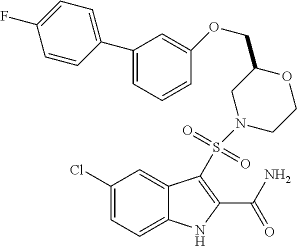 [SMILES: NC(=O)c1[nH]c2ccc(Cl)cc2c1S(=O)(=O)N1CCO[C@H](COc2cccc(-c3ccc(F)cc3)c2)C1]

¹H NMR (DMSO-d₆, 300 MHz): δ 13.01 (s, 1H), 8.20 (d, J=12 Hz, 2H), 7.88 (d, J=3 Hz, 1H), 7.68 (dd, J=9 and 3 Hz, 2H), 7.56 (d, J=9 Hz, 1H), 7.35-7.31 (m, 2H), 7.32-7.24 (m, 2H), 7.22 (d, J=9 Hz, 1H), 7.10 (s, 1H), 6.8 (dd, J=9 and 3 Hz, 1H), 4.02-3.90 (m, 2H), 3.92 (d, J=9 Hz, 1H), 3.89-3.83 (m, 1H), 3.79 (d, J=12 Hz, 1H), 3.58-3.53 (m, 2H), 2.48-2.34 (m, 2H).

MS: (m/z) 544.1 [M+H]

### Preparation of (S)-5-chloro-3-((2-(((3′-fluoro-[1,1′-biphenyl]-3-yl)oxy)methyl)morpholino)sulfonyl)-1H-indole-2-carboxamide (12)

Synthesized by similar route as represented in scheme-1 by using (3-fluorophenyl)boronic acid in step-2.

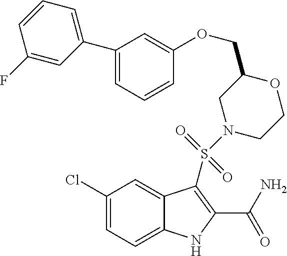 [SMILES: NC(=O)c1[nH]c2ccc(Cl)cc2c1S(=O)(=O)N1CCO[C@H](COc2cccc(-c3cccc(F)c3)c2)C1]

¹H NMR (DMSO-d₆, 300 MHz): 812.9 (s, 1H), 8.23 (d, J=12 Hz, 2H), 7.88 (d, J=1.8 Hz, 1H), 7.56-7.50 (m, 2H), 7.48-7.44 (m, 2H), 7.35-7.32 (m, 2H), 7.25 (d, J=7.8 Hz, 1H), 7.22-7.17 (m, 2H), 6.91 (dd, J=8 and 2 Hz, 1H), 4.04-3.99 (m, 2H), 3.92 (d, J=10 Hz, 1H), 3.83-3.73 (m, 1H), 3.720 (d, J=11.4 Hz, 1H), 3.58-3.49 (m, 2H), 2.48-2.34 (m, 2H).

MS: (m/z) 544.1 [M+H]

### Preparation of (S)-5-chloro-3-((2-((3-(2-fluoropyridin-4-yl)phenoxy)methyl)morpholino)sulfonyl)-1H-indole-2-carboxamide (13)

Synthesized by similar route as represented in scheme-1 by using 2-fluoropyridine-4-boronic acid in step-2.

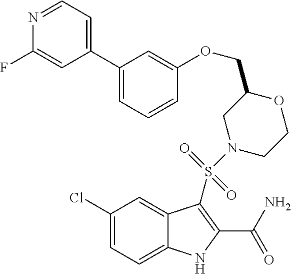 [SMILES: NC(=O)c1[nH]c2ccc(Cl)cc2c1S(=O)(=O)N1CCO[C@H](COc2cccc(-c3ccnc(F)c3)c2)C1]

¹H NMR (DMSO-d₆, 300 MHz): δ 13.02 (s, 1H), 8.27 (d, J=5.4 Hz, 1H), 8.23 (d, J=12.0 Hz, 2H), 7.89 (d, J=1.8 Hz, 1H), 7.69 (d, J=5.4 Hz, 1H), 7.57 (m, 2H), 7.41 (d, J=4.8 Hz, 2H), 7.31 (m, 2H), 7.09 (m, 1H), 3.98 (d, J=4.8 Hz, 2H), 4.08 (d, J=4.8 Hz, 2H), 3.93 (d, J=11.1 Hz, 1H), 3.85 (m, 1H), 3.73 (d, J=11.1 Hz, 1H), 3.54 (m, 2H), 2.40 (t, J=11.1 Hz, 2H).

MS: (m/z) 545 [M+H]

### Preparation of (S)-5-chloro-3-((2-(((3′-methyl-[1,1′-biphenyl]-3-yl)oxy)methyl)morpholino)sulfonyl)-1H-indole-2-carboxamide (14)

Synthesized by similar route as represented in scheme-1 by using m-tolylboronic acid in step-2.

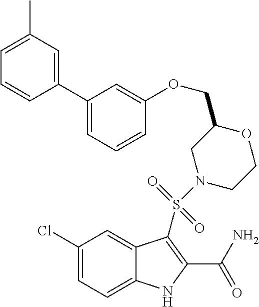 [SMILES: Cc1cccc(-c2cccc(OC[C@@H]3CN(S(=O)(=O)c4c(C(N)=O)[nH]c5ccc(Cl)cc45)CCO3)c2)c1]

¹H NMR (DMSO-d₆, 300 MHz): δ 13.02 (s, 1H), 8.24 (d, J=13 Hz, 2H), 7.8 (d, J=1.2 Hz, 1H), 7.57 (d, J=8.7 Hz, 1H), 7.45-7.40 (m, 2H), 7.33-7.28 (m, 3H), 7.20 (t, J=9 Hz, 2H), 7.10 (s, 1H), 6.88 (d, J=8 Hz, 1H), 4.02-4.01 (m, 2H), 3.9-3.8 (m, 1H), 3.75-3.70 (m, 1H), 3.69 (d, J=11 Hz, 1H), 3.55-3.50 (m, 3H), 2.39 (s, 1H), 2.3 (s, 3H).

MS: (m/z) 540.1 [M+H]

### Preparation of (S)-5-chloro-3-((2-((3-(pyridin-2-yl)phenoxy)methyl)morpholino)sulfonyl)-1H-indole-2-carboxamide (15)

Synthesized by similar route as represented in scheme-1 by using 2-pyridineboronic acid in step-2.

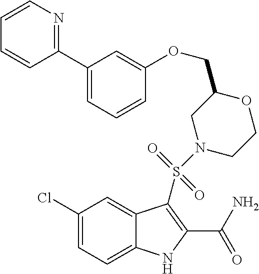 [SMILES: NC(=O)c1[nH]c2ccc(Cl)cc2c1S(=O)(=O)N1CCO[C@H](COc2cccc(-c3ccccn3)c2)C1]

¹H NMR (DMSO-d₆, 300 MHz): δ 13.00 (s, 1H), 8.64 (d, J=4.8 Hz, 1H), 8.24 (d, J=14.1 Hz, 2H), 7.96 (m, 3H), 7.65 (d, J=7.8 Hz, 1H), 7.57 (d, J=8.4 Hz, 2H), 7.38 (dd, J=15 and 38.1 Hz, 3H), 6.97 (dd, J=6.1 and 1.8 Hz, 1H), 4.05 (d, J=4.5 Hz, 2H), 3.94 (d, J=11.1 Hz, 1H), 3.85 (m, 1H), 3.73 (d, J=11.1 Hz, 1H), 3.54 (dd, J=18.6 and 11.7 Hz, 2H), 2.40 (m, 2H).

MS: (m/z) 527 [M+H]

### Preparation of (S)-5-chloro-3-((2-((3-(2-methoxypyrimidin-5-yl)phenoxy)methyl)morpholino)sulfonyl)-1H-indole-2-carboxamide (16)

Synthesized by similar route as represented in scheme-1 by using (2-methoxypyrimidin-5-yl)boronic acid in step-2.

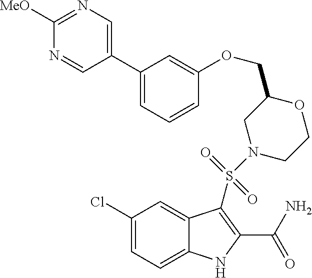 [SMILES: COc1ncc(-c2cccc(OC[C@@H]3CN(S(=O)(=O)c4c(C(N)=O)[nH]c5ccc(Cl)cc45)CCO3)c2)cn1]

¹H NMR (CDCl₃, 300 MHz): δ 10.55 (br. s, 1H), 9.04 (br. s, 1H), 8.69 (s, 2H), 8.14 (d, J=1.8 Hz, 1H), 7.50 (d, J=9.0 Hz, 1H), 7.39 (d, J=6.0 Hz, 1H), 7.37 (s, J=8.4 Hz, 1H), 7.12 (d, J=7.8, 1H), 6.97 (m, 1H), 6.89 (dd, J=8.1, 1.8 Hz, 1H), 6.17 (br. s, 1H), 4.06 (s, 3H), 4.05-3.59 (m, 7H), 2.62 (dt, J=11.4 and 3.0 Hz, 1H), 2.51 (t, J=9.9 Hz, 1H),

MS: (m/z) 558 [M+H]

### Preparation of (S)-5-chloro-3-((2-((3-(6-ethoxypyridin-3-yl)phenoxy)methyl)morpholino)sulfonyl)-1H-indole-2-carboxamide (17)

Synthesized by similar route as represented in scheme-1 by using 2-ethoxypyridine-5-boronic acid in step-2.

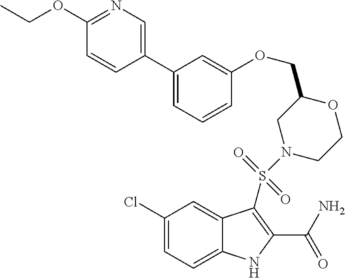 [SMILES: CCOc1ccc(-c2cccc(OC[C@@H]3CN(S(=O)(=O)c4c(C(N)=O)[nH]c5ccc(Cl)cc45)CCO3)c2)cn1]

¹H NMR (CDCl₃, 300 MHz): δ 10.41 (br. s, 1H), 9.05 (br. s, 1H), 8.33 (d, J=2.1 Hz, 1H), 8.14 (d, J=1.5 Hz, 1H), 7.76 (dd, J=8.7 and 2.7 Hz, 1H), 7.48 (d, J=9.0 Hz, 1H), 7.37 (dd, J=8.7 and 1.8 Hz, 1H), 7.34 (d, J=8.1 Hz, 1H), 7.13 (d, J=7.8 Hz, 1H), 7.02 (s, 1H), 6.84 (dd, J=8.1 and 1.8 Hz, 1H), 6.79 (d, J=9.0 Hz, 1H), 6.06 (br. s, 1H), 4.42 (q, J=6.9 Hz, 2H), 4.08-3.94 (m, 4H), 3.80-3.74 (m, 2H), 3.59 (d, J=11.1 Hz, 1H), 2.65 (dt, J=11.4 and 3.0 Hz, 1H), 2.51 (t, J=9.9 Hz, 1H), 1.44 (t, J=6.9 Hz, 3H).

MS: (m/z) 571 [M+H]

### Preparation of (S)-5-chloro-3-((2-((3-(2-methoxypyridin-4-yl)phenoxy)methyl)morpholino)sulfonyl)-1H-indole-2-carboxamide (18)

Synthesized by similar route as represented in scheme-1 by using 2-methoxypyridin-4-boronic acid in step-2.

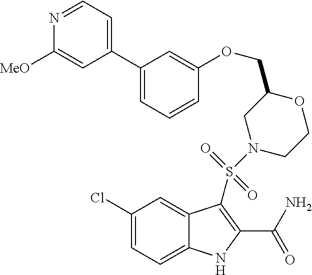 [SMILES: COc1cc(-c2cccc(OC[C@@H]3CN(S(=O)(=O)c4c(C(N)=O)[nH]c5ccc(Cl)cc45)CCO3)c2)ccn1]

¹H NMR (CDCl₃, 300 MHz): δ 10.35 (br. s, 1H), 9.04 (br. s, 1H), 8.20 (d, J=5.4 Hz, 1H), 8.14 (d, J=1.5 Hz, 1H), 7.48 (d, J=8.7 Hz, 1H), 7.32 (d, J=7.8 Hz, 1H), 7.38 (d, J=2.1 Hz, 1H), 7.22 (d, J=7.8, 1H), 7.07-7.05 (m, 2H), 6.91-6.88 (m, 2H), 6.01 (s, 1H), 4.08-3.94 (m, 7H), 3.81-3.74 (m, 2H), 3.64 (d, J=12.0 Hz, 1H), 2.59 (dt, J=11.4 and 3.0 Hz, 1H), 2.51 (t, J=9.9 Hz, 1H).

MS: (m/z) 557 [M+H]

### Preparation of (S)-5-chloro-3-((2-((3-(2-methylpyridin-4-yl)phenoxy)methyl)morpholino)sulfonyl)-1H-indole-2-carboxamide (19)

Synthesized by similar route as represented in scheme-1 by using 2-methylpyridine-4-boronic acid in step-2.

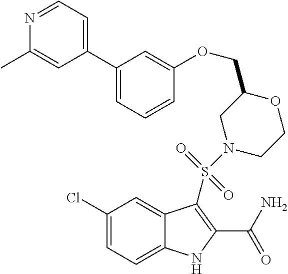 [SMILES: Cc1cc(-c2cccc(OC[C@@H]3CN(S(=O)(=O)c4c(C(N)=O)[nH]c5ccc(Cl)cc45)CCO3)c2)ccn1]

¹H NMR (CDCl₃, 300 MHz): δ 10.42 (br. s, 1H), 9.07 (br. s, 1H), 8.55 (d, J=3.0 Hz, 1H), 8.17 (d, J=0.9 Hz, 1H), 7.49 (d, J=5.1 Hz, 1H), 7.40-7.35 (m, 3H), 7.30 (d, J=3.3 Hz, 1H), 7.25 (d, J=4.8 Hz, 1H), 7.12 (s, 1H), 6.94 (dd, J=4.8 and 1.5 Hz, 1H), 6.04 (br. s, 1H), 4.08-3.96 (m, 4H), 3.83-3.76 (m, 2H), 3.64 (d, J=11.1 Hz, 1H), 2.64 (s, 3H), 2.62 (dt, J=11.4 and 3.0 Hz, 1H), 2.52 (t, J=9.9 Hz, 1H).

MS: (m/z) 541 [M+H]

### Preparation of (S)-5-chloro-3-((2-(((6-methyl-[1,1′-biphenyl]-3-yl)oxy)methyl)morpholino)sulfonyl)-1H-indole-2-carboxamide (20)

Synthesized by similar route as represented in scheme-1 by using 3-bromo-4-methylphenol in step-1 and phenylboronic acid in step-2.

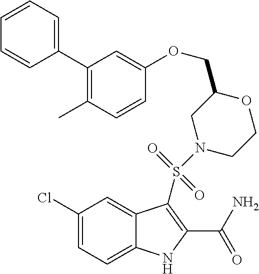 [SMILES: Cc1ccc(OC[C@@H]2CN(S(=O)(=O)c3c(C(N)=O)[nH]c4ccc(Cl)cc34)CCO2)cc1-c1ccccc1]

¹H NMR (CDCl₃, 300 MHz): δ 10.38 (br. s, 1H), 9.03 (br. s, 1H), 8.13 (d, J=1.5 Hz, 1H), 7.47 (d, J=8.7 Hz, 1H), 7.41-7.27 (m, 6H), 7.15 (d, J=8.1 Hz, 1H), 6.77-6.72 (m, 2H), 5.99 (br. s, 1H), 3.98-3.84 (m, 4H), 3.77-3.68 (m, 2H), 3.60 (d, J=11.4 Hz, 1H), 2.60 (dt, J=11.4 and 3.3 Hz, 1H), 2.45 (t, J=11.1 Hz, 1H), 2.01 (s, 3H).

MS: (m/z) 538 [M−H]

### Preparation of (S)-5-chloro-3-((2-(((2′-fluoro-6-methyl-[1,1′-biphenyl]-3-yl)oxy)methyl)morpholino)sulfonyl)-1H-indole-2-carboxamide (21)

Synthesized by similar route as represented in scheme-1 by using 3-bromo-4-methylphenol in step-1 and 2-fluorophenylboronic acid in step-2.

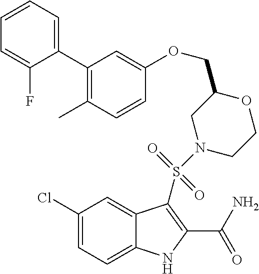 [SMILES: Cc1ccc(OC[C@@H]2CN(S(=O)(=O)c3c(C(N)=O)[nH]c4ccc(Cl)cc34)CCO2)cc1-c1ccccc1F]

¹H NMR (CDCl₃, 300 MHz): δ 10.39 (br. s, 1H), 9.03 (br. s, 1H), 8.13 (d, J=1.5 Hz, 1H), 7.47 (d, J=9.0 Hz, 1H), 7.36 (dd, J=8.7 and 1.8 Hz, 1H), 7.29-7.17 (m, 1H), 7.21-7.08 (m, 4H), 6.84 (dd, J=8.4 and 2.7 Hz, 1H), 6.72 (d, J=2.7 Hz, 1H), 5.99 (br. s, 1H), 3.99-3.84 (m, 4H), 3.77-3.68 (m, 2H), 3.60 (d, J=11.7 Hz, 1H), 2.57 (dt, J=11.4 and 3.3 Hz, 1H), 2.46 (t, J=9.6 Hz, 1H), 2.01 (s, 3H).

MS: (m/z) 558 [M+H]

### Preparation of (S)-5-chloro-3-((2-((3-(2-fluoropyridin-3-yl)-4-methylphenoxy)methyl)morpholino)sulfonyl)-1H-indole-2-carboxamide (22)

Synthesized by similar route as represented in scheme-1 by using 3-bromo-4-methylphenol in step-1 and 2-fluoro-pyridine-3-boronic acid in step-2.

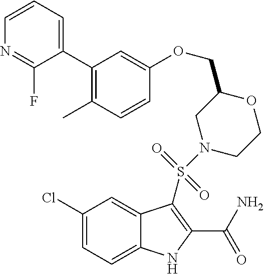 [SMILES: Cc1ccc(OC[C@@H]2CN(S(=O)(=O)c3c(C(N)=O)[nH]c4ccc(Cl)cc34)CCO2)cc1-c1cccnc1F]

¹H NMR (CDCl₃, 300 MHz): δ 10.39 (br. s, 1H), 9.03 (br. s, 1H), 8.24 (d, J=4.5 Hz, 1H), 8.12 (s, 1H), 7.66 (m, 1H), 7.47 (d, J=8.7 Hz, 1H), 7.36 (dd, J=8.7 and 1.8 Hz, 1H), 7.27-7.17 (m, 1H), 7.18 (d, J=8.4 Hz, 1H), 6.84 (dd, J=8.4 and 2.7 Hz, 1H), 6.70 (d, J=2.7 Hz, 1H), 6.01 (br. s, 1H), 3.99-3.86 (m, 4H), 3.77-3.68 (m, 2H), 3.60 (d, J=11.7 Hz, 1H), 2.57 (dt, J=11.0 and 3.3 Hz, 1H), 2.45 (t, J=9.3 Hz, 1H), 2.01 (s, 3H).

MS: (m/z) 559 [M+H]

### Preparation of (S)-5-chloro-3-((2-(((2′-methoxy-6-methyl-[1,1′-biphenyl]-3-yl)oxy)methyl)morpholino)sulfonyl)-1H-indole-2-carboxamide (23)

Synthesized by similar route as represented in scheme-1 by using 3-bromo-4-methylphenol in step-1 and 2-methoxyphenylboronic acid in step-2.

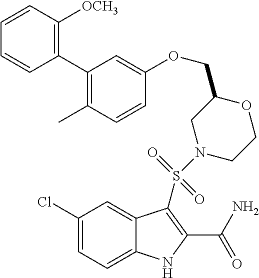 [SMILES: COc1ccccc1-c1cc(OC[C@@H]2CN(S(=O)(=O)c3c(C(N)=O)[nH]c4ccc(Cl)cc34)CCO2)ccc1C]

¹H NMR (CDCl₃, 300 MHz): δ 10.32 (br. s, 1H), 8.99 (br. s, 1H), 8.12 (d, J=1.0 Hz, 1H), 7.45 (d, J=8.7 Hz, 1H), 7.37 (m, 2H), 7.13-7.09 (m, 2H), 7.01 (d, J=7.2 Hz, 1H), 6.96 (d, J=8.7 Hz, 1H), 6.75 (dd, J=8.4 and 2.7 Hz, 1H), 6.69 (d, J=2.7 Hz, 1H), 5.96 (br. s, 1H), 3.99-3.85 (m, 5H), 3.75 (s, 3H), 3.77-3.68 (m, 1H), 3.50 (d, J=9.6 Hz, 1H), 2.60 (dt, J=11.7 and 3.3 Hz, 1H), 2.45 (dd, J=10.8 and 9.0 Hz, 1H), 2.04 (s, 3H).

MS: (m/z) 570 [M+H]

### Preparation of (S)-5-chloro-3-((2-(((2′,6-dimethyl-[1,1′-biphenyl]-3-yl)oxy)methyl)morpholino)sulfonyl)-1H-indole-2-carboxamide (24)

Synthesized by similar route as represented in scheme-1 by using 3-bromo-4-methylphenol in step-1 and o-tolylboronic acid in step-2.

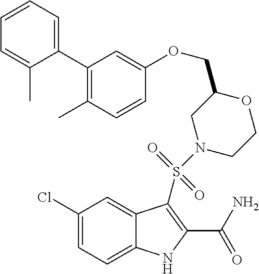 [SMILES: Cc1ccccc1-c1cc(OC[C@@H]2CN(S(=O)(=O)c3c(C(N)=O)[nH]c4ccc(Cl)cc34)CCO2)ccc1C]

¹H NMR (CDCl₃, 300 MHz): δ 10.42 (br. s, 1H), 9.04 (br. s, 1H), 8.12 (d, J=1.0 Hz, 1H), 7.47 (d, J=9.0 Hz, 1H), 7.37 (dd, J=8.7 and 1.5 Hz, 1H), 7.25-7.17 (m, 2H), 7.14 (d, J=8.4 Hz, 1H), 7.07 (d, J=6.9 Hz, 1H), 6.76 (m, 1H), 6.61 (t, J=2.4 Hz, 1H), 6.01 (br. s, 1H), 3.99-3.83 (m, 5H), 3.77-3.68 (m, 2H), 3.60 (d, J=11.7 Hz, 1H), 2.58 (dt, J=11.1 and 3.0 Hz, 1H), 2.45 (t, J=10.2 Hz, 1H), 2.03 (s, 3H), 1.95 (s, 3H).

MS: (m/z) 554 [M+H]

### Preparation of (S)-5-chloro-3-((2-((3-(6-methoxypyridin-3-yl)-4-methylphenoxy)methyl)morpholino)sulfonyl)-1H-indole-2-carboxamide (25)

Synthesized by similar route as represented in scheme-1 by using 3-bromo-4-methylphenol in step-1 and 2-methoxypyridin-5-ylboronic acid in step-2.

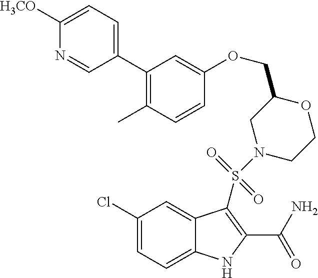 [SMILES: COc1ccc(-c2cc(OC[C@@H]3CN(S(=O)(=O)c4c(C(N)=O)[nH]c5ccc(Cl)cc45)CCO3)ccc2C)cn1]

¹H NMR (300 MHz, CDCl₃): δ 10.44 (br. s, 1H), 9.04 (br. s, 1H), 8.12 (d, J=1.8 Hz, 1H), 8.09 (d, J=2.1 Hz, 1H), 7.53 (dd, J=8.4 and 2.4 Hz, 1H), 7.47 (d, J=9.0 Hz, 1H), 7.36 (dd, J=8.7 and 2.7 Hz, 1H), 7.16 (d, J=8.4 Hz, 1H), 6.80-6.74 (m, 2H), 6.69 (d, J=2.4 Hz, 1H), 6.05 (br. s, 1H), 3.99-3.87 (m, 7H), 3.77-3.73 (m, 2H), 3.62 (d, J=11.4 Hz, 1H), 2.58 (dt, J=11.4 and 3.3 Hz, 1H), 2.46 (t, J=10.2 Hz, 1H), 2.17 (s, 3H).

MS: (m/z) 571 [M+H]

### Preparation of (S)-5-chloro-3-((2-((4-fluoro-3-(pyridin-3-yl)phenoxy)methyl)morpholino)sulfonyl)-1H-indole-2-carboxamide (26)

Synthesized by similar route as represented in scheme-1 by using 3-bromo-4-fluorophenol in step-1 and pyridine-3-boronic acid in step-2.

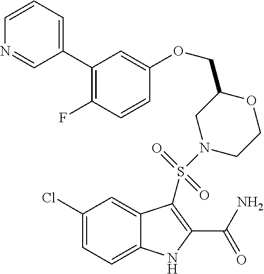 [SMILES: NC(=O)c1[nH]c2ccc(Cl)cc2c1S(=O)(=O)N1CCO[C@H](COc2ccc(F)c(-c3cccnc3)c2)C1]

¹H NMR (DMSO-d₆, 300 MHz): δ 13.02 (s, 1H), 8.72 (s, 1H), 8.58 (m, 1H), 8.23 (d, J=10.8 Hz, 2H), 7.96 (m, 1H), 7.88 (d, J=1.8 Hz, 1H), 7.56 (d, J=8.7 Hz, 1H), 7.5 (dd, J=7.8 and 4.8 Hz, 1H), 7.34 (dd, J=8.7 and 1.8 Hz, 1H), 7.23 (t, J=9.3 Hz, 1H), 7.09 (dd, J=6.3 and 3.0 Hz, 1H), 6.98 (m, 1H), 4.03 (d, J=4.8 Hz, 2H), 3.92 (d, J=11.1 Hz 1H), 3.80 (s, 1H), 3.70 (d, J=10.8 Hz, 1H), 3.52 (m, 2H), 2.39 (t, J=10.8 Hz, 2H).

MS: (m/z) 545 [M+H]

### Preparation of (S)-5-chloro-3-((2-((4-fluoro-3-(pyrimidin-5-yl)phenoxy)methyl)morpholino)sulfonyl)-1H-indole-2-carboxamide (27)

Synthesized by similar route as represented in scheme-1 by using 3-bromo-4-fluorophenol in step-1 and pyrimidin-5-ylboronic acid in step-2.

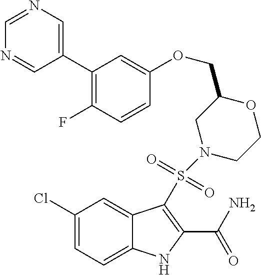

¹H NMR (DMSO-d₆, 300 MHz): δ 13.02 (s, 1H), 9.2 (s, 1H), 9.0 (d, J=1.2 Hz, 2H), 8.23 (d, J=10.5 Hz, 2H), 7.88 (d, J=1.5 Hz, 1H), 7.56 (d, J=8.7 Hz, 1H), 7.34 (dd, J=8.4 and 2.1 Hz, 1H), 7.28 (d, J=9.9 Hz, 1H), 7.20 (dd, J=6.3 and 3.0 Hz, 1H), 7.03 (m, 1H), 4.00 (d, J=4.8 Hz, 2H), 3.92 (d, J=11.4 Hz, 1H), 3.80 (m, 1H), 3.70 (d, J=11.1 Hz, 1H), 3.54 (m, 2H), 2.39 (t, J=11.1 Hz, 2H).

MS: (m/z) 546 [M+H]

### Preparation of (S)-5-chloro-3-((2-((4-fluoro-3-(pyridin-4-yl)phenoxy)methyl)morpholino)sulfonyl)-1H-indole-2-carboxamide (28)

Synthesized by similar route as represented in scheme-1 by using 3-bromo-4-fluorophenol in step-1 and pyridine-4-boronic acid in step-2.

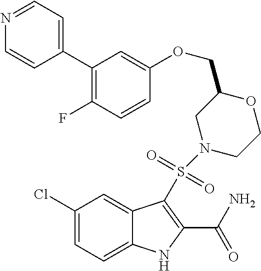

¹H NMR (DMSO-d₆, 300 MHz): δ 13.02 (s, 1H), 8.65 (d, J=6.0 Hz, 2H), 8.23 (d, J=12.6 Hz, 2H), 7.88 (d, J=1.8 Hz, 1H), 7.56 (m, 3H), 7.35 (dd, J=9.0 and 1.8 Hz, 1H), 7.25 (t, J=9.3 Hz, 1H), 7.10 (dd, J=6.3 and 3.0 Hz, 1H), 7.00 (m, 1H), 4.03 (d, J=4.8 Hz, 2H), 3.92 (d, J=10.8 Hz, 1H), 3.80 (m, 1H), 3.70 (d, J=11.4 Hz, 1H), 3.52 (m, 2H), 2.44 (t, J=11.7 Hz, 2H).

MS: (m/z) 545 [M+H]

### Preparation of (S)-5-chloro-3-((2-((4-fluoro-3-(6-methoxypyridin-3-yl)phenoxy)methyl)morpholino)sulfonyl)-1H-indole-2-carboxamide (29)

Synthesized by similar route as represented in scheme-1 by using 3-bromo-4-fluorophenol in step-1 and 2-methoxypyridine-5-boronic acid in step-2.

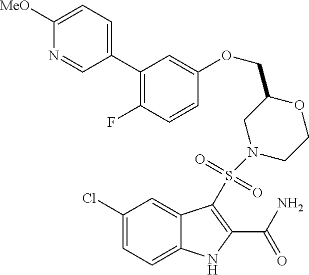

¹H NMR (DMSO-d₆, 300 MHz): δ 13.02 (s, 1H), 8.33 (s, 1H), 8.23 (d, J=11.7 Hz, 2H), 7.88 (d, J=2.1 Hz, 2H), 7.56 (d, J=8.7 Hz, 1H), 7.35 (dd, J=10.8 and 1.8 Hz, 1H), 7.23 (t, J=9.3 Hz, 1H), 7.04 (dd, J=6.3 and 3.0 Hz, 1H), 6.98 (t, J=3.9 Hz, 2H), 4.01 (d, J=4.8 Hz, 2H), 3.92 (s, 1H), 3.87 (s, 3H), 3.80 (s, 1H), 3.70 (d, J=11.4 Hz, 1H), 3.54 (m, 2H), 2.4 (t, J=12.0 Hz, 2H).

MS: (m/z) 575 [M+H]

### Preparation of (S)-5-chloro-3-((2-((4-fluoro-3-(2-methylpyridin-4-yl)phenoxy)methyl)morpholino)sulfonyl)-1H-indole-2-carboxamide (30)

Synthesized by similar route as represented in scheme-1 by using 3-bromo-4-fluorophenol in step-1 and 2-methylpyridine-4-boronic acid in step-2.

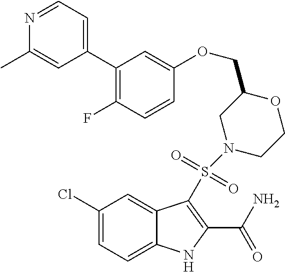

¹H NMR (DMSO-d₆, 300 MHz): δ 13.02 (s, 1H), 8.52 (d, J=5.4 Hz, 1H), 8.25 (d, J=13.2 Hz, 2H), 7.90 (d, J=2.1 Hz, 1H), 7.58 (d, J=8.7 Hz, 1H), 7.44 (s, 1H), 7.37 (m, 2H), 7.25 (t, J=9.0 Hz, 1H), 7.1 (dd, J=6.3 and 3.3 Hz, 1H), 6.98 (m, 1H), 4.05 (d, J=4.8 Hz, 2H), 3.94 (d, J=11.1 Hz, 1H), 3.84 (m, 1H), 3.72 (d, J=11.7 Hz, 1H), 3.54 (m, 2H), 2.5 (s, 3H), 2.4 (t, J=12.0 Hz, 2H).

MS: (m/z) 559 [M+H]

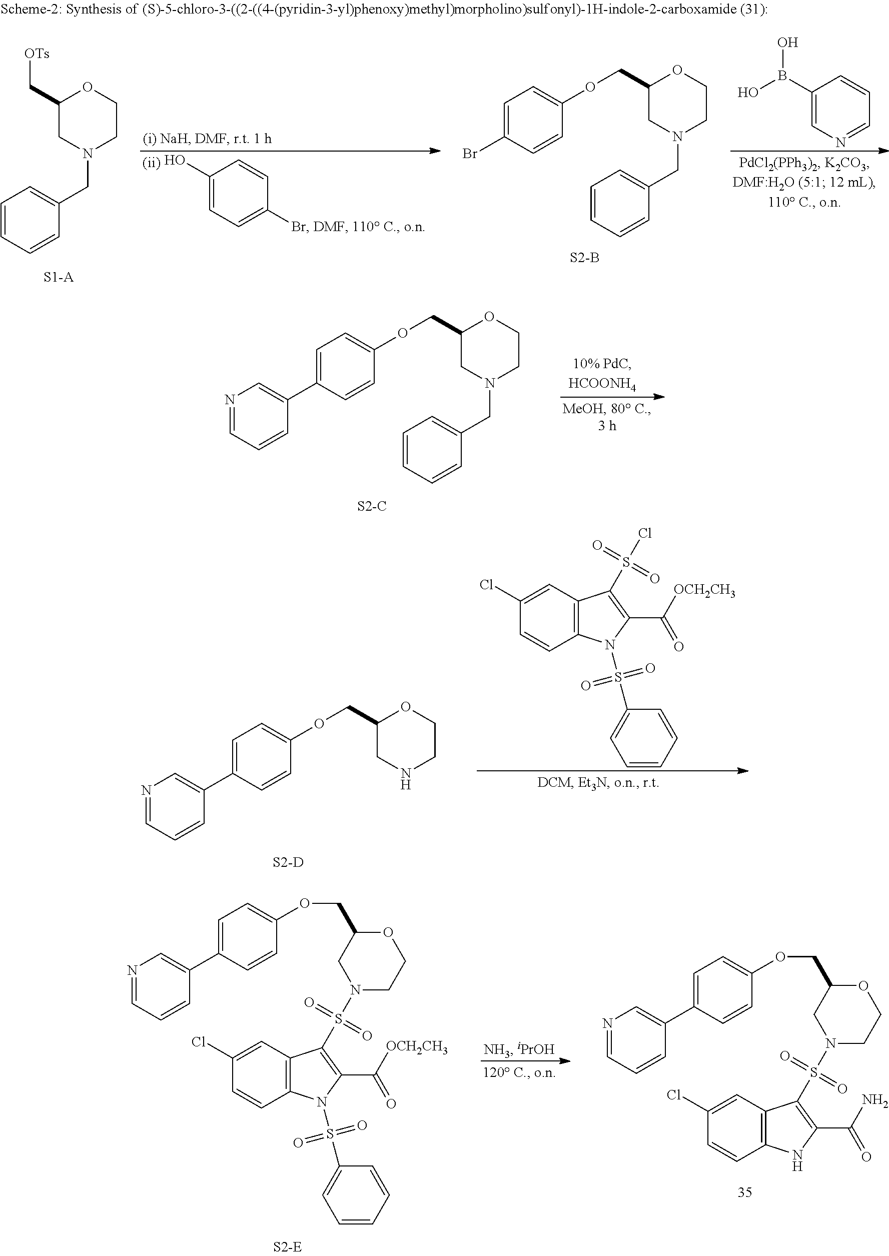

### Step-1. Preparation of (S)-4-benzyl-2-((4-bromophenoxy)methyl)morpholine (S2-B)

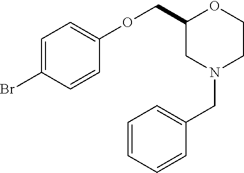

A solution of p-bromo phenol (1 eq) in DMF was added to a mixture of NaH (1 eq) in DMF at room temperature. The reaction mixture was initially stirred at ambient temperature for 10 minutes. The sodium alkoxide solution was then charged with (S)-(4-benzylmorpholin-2-yl)methyl 4-methylbenzenesulfonate (1 eq) in DMF and the resulting reaction mixture was heated at 110° C. for 2 h. The solution was concentrated under reduced pressure; residue obtained was diluted with ethyl acetate (50 mL) and washed with water (10 mL) and brine (5 mL). The combined organic layers were dried over anhydrous Na₂SO₄, filtered, concentrated in vacuo, and the residue obtained was purified by flash column chromatography (200-400 mesh size silica gel, 40% EtOAc/pet. ether gradient elution) to yield (S)-4-benzyl-2-((4-bromophenoxy)methyl)morpholine as a gummy oil.

¹H NMR (CDCl₃, 300 MHz): δ 7.44 (s, 1H), 7.39 (s, 1H), 7.33-7.22 (m, 5H), 6.92 (s, 1H), 6.86 (s, 1H), 3.9 (d, J=4.8 Hz, 1H), 3.82-3.81 (m, 2H), 3.57 (dd, J=11.1 and 2.4 Hz, 1H), 3.49 (s, 2H), 3.34 (s, 1H), 2.81 (d, J=11.4 Hz, 1H), 2.63 (d, J=11.1 Hz, 1H), 2.13 (dt, J=11.4 and 3.3 Hz, 1H), 2.00 (t, J=11.4 Hz, 1H).

### Step-2. Preparation of (S)-4-benzyl-2-((4-(pyridin-3-yl)phenoxy)methyl)morpholine (S2-C)

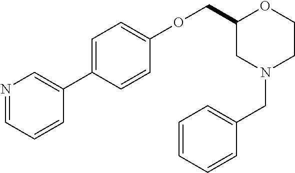

A mixture of (S)-4-benzyl-2-((4-bromophenoxy)methyl)morpholine (1 eq), phenylboronic acid (1.2 eq) and potassium carbonate (1.4 eq) in DMF:H₂O (2:1) (12 mL) were sonicated for 2-3 minutes and stirred under argon atmosphere at an ambient temperature. The dichlorobis(triphenylphosphine)Palladium(II) (0.1 eq) was added to the reaction mixture and stirred at 120° C. until complete consumption of the starting materials was indicated by TLC analysis (approximately 3 h). The reaction was diluted with EtOAc (50 mL) and filtered through a celite bed. The filtrate was washed with water (20 mL), brine (5 mL). The organic layer was dried over anhydrous Na₂SO₄, filtered, concentrated in vacuo and the residue obtained was purified by flash column chromatography (200-400 mesh size silica gel, 30% ethyl acetate/pet. ether, gradient elution) to yield (S)-4-benzyl-2-((4-(pyridin-3-yl)phenoxy)methyl)morpholine as a gummy oil.

### Step-3. Preparation of (S)-2-((4-(pyridin-3-yl)phenoxy)methyl)morpholine (S2-D)

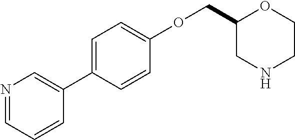

To a methanolic solution (20 mL) of (S)-4-benzyl-2-((4-(pyridin-3-yl)phenoxy)methyl)morpholine (1 eq) and ammonium formate (5 eq) in a two-necked round bottom flask Pd/C was added under argon atmosphere. The reaction mixture was refluxed till complete consumption of starting material as indicated by TLC (3 h). The reaction was filtered through celite bed and filtrate obtained was concentrated in vacuo and the residue obtained was purified by flash column chromatography (200-400 mesh size silica gel, 10% methanol/dichloromethane, gradient elution) to yield (S)-2-((4-(pyridin-3-yl)phenoxy)methyl)morpholine as a gummy oil.

### Step-4. Preparation of (S)-ethyl 5-chloro-1-(phenylsulfonyl)-3-((2-((4-(pyridin-3-yl)phenoxy)methyl)morpholino)sulfonyl)-1H-indole-2-carboxylate (S2-E)

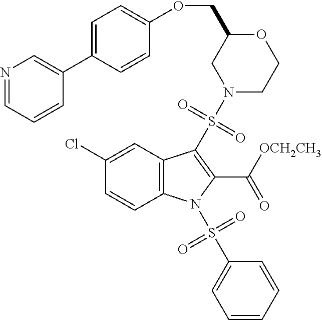

To a solution of (S)-2-((4-(pyridin-3-yl)phenoxy)methyl)morpholine (1 eq) in dichloromethane (10 mL), triethyl amine (5 eq) was added and the reaction mixture was stirred under argon atmosphere. Ethyl 5-chloro-3-(chlorosulfonyl)-1-(phenylsulfonyl)-1H-indole-2-carboxylate (1.2 eq) was added to reaction mixture and the resulting reaction mixture was stirred for approximately 16 h at ambient temperature. The reaction was concentrated in vacuo, crude material was adsorbed on silica gel and subjected to column chromatography (200-400 mesh size) using 5% methanol/dichloromathane as gradient eluent to furnish (S)-ethyl 5-chloro-1-(phenylsulfonyl)-3-((2-((4-(pyridin-3-yl)phenoxy)methyl)morpholino)sulfonyl)-1H-indole-2-carboxylate as a yellowish solid.

¹H NMR (CDCl₃, 300 MHz): δ 8.84-8.60 (m, 3H), 8.11 (d, J=7.5 Hz, 2H), 8.00 (d, J=9 Hz, 1H), 7.92 (m, 2H), 7.67-7.52 (m, 3H), 7.47-7.39 (m, 3H), 7.11-7.01 (m, 2H), 4.62-4.548 (q, J=7.2 Hz, 2H), 4.10-3.91 (m, 5H), 3.75-3.71 (m, 2H), 2.81-2.70 (m, 2H), 1.50-1.45 (t, J=7.2 Hz, 3H).

MS (m/z): 696 [M⁺]

### Step-5. Preparation of (S)-5-chloro-3-((2-((4-(pyridin-3-yl)phenoxy)methyl)morpholino)sulfonyl)-1H-indole-2-carboxamide (31)

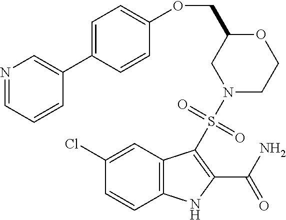

To a solution of (S)-ethyl 5-chloro-1-(phenylsulfonyl)-3-((2-((4-(pyridin-3-yl)phenoxy)methyl)morpholino)sulfonyl)-1H-indole-2-carboxylate (1 eq) in isopropyl alcohol (20 mL) at 0° C. ammonia gas was bubbled for 15 minutes. The resulting solution was sealed in a seal-tube and placed in an oil bath at 120° C. for 16 h. The solvent was removed under reduced pressure. Crude product was subjected to flash column chromatography (200-400 mesh size silica gel, 10% methanol/dichloromethane, gradient elution) to afford (S)-5-chloro-3-((2-((4-(pyridin-3-yl)phenoxy)methyl)morpholino)sulfonyl)-1H-indole-2-carboxamide (31) as a yellowish solid.

¹H NMR (CDCl₃+DMSO-d₆, 300 MHz): δ 12.60 (s, 1H), 8.60-8.50 (m, 2H), 8.35 (s, 1H), 7.89-7.66 (m, 2H), 7.42 (d, J=8.7 Hz, 1H), 7.38-7.25 (m, 2H), 7.19 (s, 1H), 7.11 (d, J=8.7 Hz, 1H), 6.90-6.77 (m, 2H), 6.41 (s, 1H), 3.86-3.75 (m, 3H), 3.58 (d, J=22.8 Hz, 2H), 3.06 (s, 1H), 2.40-2.21 (m, 3H).

MS (m/z): 527 [M+H]

### Preparation of (S)-5-chloro-3-((2-((4-(pyridin-4-yl)phenoxy)methyl)morpholino)sulfonyl)-1H-indole-2-carboxamide (32)

Synthesized by similar route as represented in scheme-2 by using pyridine-4-boronic acid in step-2.

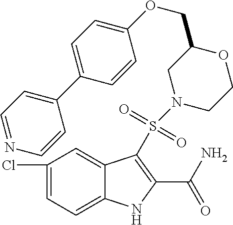 [SMILES: NC(=O)c1[nH]c2ccc(Cl)cc2c1S(=O)(=O)N1CCO[C@H](COc2ccc(-c3ccncc3)cc2)C1]

¹H NMR (DMSO-d₆, 300 MHz): δ 13.03 (s, 1H), 8.58 (d, J=6 Hz, 2H), 8.31-8.22 (m, 2H), 7.97 (s, 1H), 7.76 (d, J=8.7 Hz, 2H), 7.67 (d, J=6 Hz, 2H), 7.59 (d, J=8.7 Hz, 1H), 7.38 (d, J=8.7 Hz, 1H), 7.05 (d, J=8.7 Hz, 2H), 4.06 (d, J=4.5 Hz, 2H), 3.95 (d, J=11.1 Hz, 1H), 3.86 (s, 1H), 3.74 (d, J=11.1 Hz, 1H), 3.60-3.52 (m, 2H), 2.43-2.36 (m, 2H).

MS (m/z): 527 [M+H]

### Preparation of (S)-3-((2-(([1,1′-biphenyl]-4-yloxy)methyl)morpholino)sulfonyl)-5-chloro-1H-indole-2-carboxamide (33)

Synthesized by similar route as represented in scheme-2 by using phenylboronic acid in step-2.

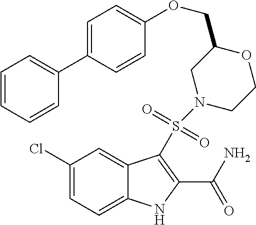 [SMILES: NC(=O)c1[nH]c2ccc(Cl)cc2c1S(=O)(=O)N1CCO[C@H](COc2ccc(-c3ccccc3)cc2)C1]

¹H NMR (DMSO-d₆, 300 MHz): δ 13.05 (s, 1H), δ 8.26 (d, J=13.5 Hz, 2H), 7.91 (s, 1H), 7.84 (d, J=7.5 Hz, 1H), 6.90 (m, 1H), 7.61-6.97 (m, 9H), 7.81-7.55 (m, 5H), 7.45-7.26 (m, 4H), 4.02-4.01 (m, 2H), 3.95 (m, 1H), 3.91-3.71 (m, 2H), 3.60-3.52 (m, 2H), 2.44-2.37 (m, 2H).

MS (m/z): 526 [M+H]

### Preparation of (S)-5-chloro-3-((2-((4-(6-(dimethylamino)pyridin-3-yl)phenoxy)methyl)morpholino)sulfonyl)-1H-indole-2-carboxamide (34)

Synthesized by similar route as represented in scheme-2 by using (6-(dimethylamino)pyridin-3-yl)boronic acid in step-2.

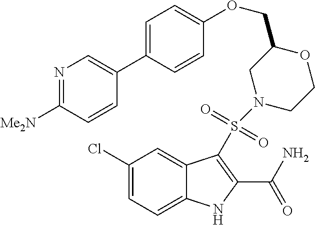 [SMILES: CN(C)c1ccc(-c2ccc(OC[C@@H]3CN(S(=O)(=O)c4c(C(N)=O)[nH]c5ccc(Cl)cc45)CCO3)cc2)cn1]

¹H NMR (CDCl₃, 300 MHz): δ 9.07 (s, 1H), 8.38-8.37 (d, J=2.1 Hz, 1H), 8.16 (s, 1H), 7.97-7.94 (d, J=7.2 Hz, 2H), 7.66-7.65 (d, J=2.4 Hz, 1H), 7.63-7.59 (dd, J=2.4 Hz, 1H), 7.56-7.54 (d, J=7.5 Hz, 2H), 7.51 (s, 1H), 7.48-7.43 (d, J=8.7 Hz, 2H), 7.38-7.37 (d, J=1.8 Hz, 1H), 6.92-6.90 (d, J=8.4 Hz, 1H), 6.60-6.57 (d, J=8.7 Hz, 1H), 4.05-3.90 (m, 4H), 3.82-3.73 (m, 2H), 3.64-3.60 (d, J=11.7 Hz, 1H), δ 3.13 (s, 6H), 2.64 (m, 1H), 2.51-2.44 (m, 1H).

MS (m/z): 570 [M+H]

### Preparation of (S)-5-chloro-3-((2-((4-(6-(trffluoromethyl)pyridin-3-yl)phenoxy)methyl)morpholino)sulfonyl)-1H-indole-2-carboxamide (35)

Synthesized by similar route as represented in scheme-2 by using (6-(trifluoromethyl)pyridin-3-yl)boronic acid in step-2.

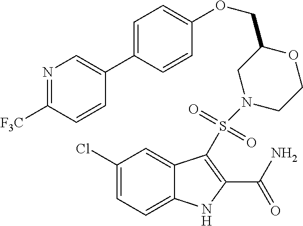 [SMILES: NC(=O)c1[nH]c2ccc(Cl)cc2c1S(=O)(=O)N1CCO[C@H](COc2ccc(-c3ccc(C(F)(F)F)nc3)cc2)C1]

¹H NMR (CDCl₃+DMSO-d₆, 300 MHz,): δ 8.84 (d, J=9.3, 2H), 8.06 (s, 1H), 7.96 (d, J=7.8, 1H), 7.69 (d, J=8.1, 1H), 7.54-7.46 (m, 3H), 7.25 (s, 1H), 6.96 (d, J=8.4, 2H), 6.39 (s, 1H), 4.01-3.89 (m, 4H), 3.74-3.67 (m, 2H), 3.56 (d, J=11.7, 1H), 2.55-2.29 (m, 2H).

MS (m/z): 595 [M+H]

### Preparation of (S)-5-chloro-3-((2-(((2′-methoxy-[1,1′-biphenyl]-4-yl)oxy)methyl)morpholino)sulfonyl)-1H-indole-2-carboxamide (36)

Synthesized by similar route as represented in scheme-2 by using 2-methoxyphenylboronic acid in step-2.

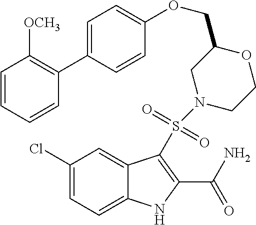 [SMILES: COc1ccccc1-c1ccc(OC[C@@H]2CN(S(=O)(=O)c3c(C(N)=O)[nH]c4ccc(Cl)cc34)CCO2)cc1]

¹H NMR (CDCl₃, 300 MHz): δ 10.6 (s, 1H), 9.05 (s, 1H), 8.16 (s, 1H), 7.50-7.44 (m, 3H), 7.39-7.34 (m, 1H), 7.31 (s, 1H), 7.05-6.97 (m, J=8.1, 7.8 and 7.2, 2H), 6.92 (d, J=9.3 Hz, 2H), 6.08 (s, 1H), 4.15-3.91 (m, 3H), 3.82-3.76 (m, 4H), 3.64 (d, J=11.4 Hz, 1H), 2.61 (m, 1H), 2.51 (m, 1H).

MS (m/z): 556 [M+H]

### Preparation of (S)-5-chloro-3-((2-((4-(6-methoxypyridin-3-yl)phenoxy)methyl)morpholino)sulfonyl)-1H-indole-2-carboxamide (37)

Synthesized by similar route as represented in scheme-2 by using (6-methoxypyridin-3-yl)boronic acid in step-2.

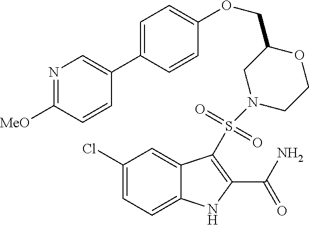 [SMILES: COc1ccc(-c2ccc(OC[C@@H]3CN(S(=O)(=O)c4c(C(N)=O)[nH]c5ccc(Cl)cc45)CCO3)cc2)cn1]

¹H NMR (CDCl₃+DMSO-d₆, 300 MHz): δ 8.80 (s, 1H), 8.27 (s, 1H), 8.06 (s, 1H), 7.70 (d, J=8.4 Hz, 1H), 7.54 (d, J=11.7 Hz, 1H), 7.38 (d, J=8.4 Hz, 2H), 7.25 (s, 1H), 6.90 (d, J=8.4 Hz, 2H), 6.76 (d, J=8.4 Hz, 1H), 6.40 (s, 1H), 3.92 (m, 5H), 3.74 (m, 2H), 3.55 (d, J=11.4 Hz, 1H), 2.54-2.32 (m, 2H).

MS (m/z): 555 [M−H]

### Preparation of (S)-5-chloro-3-((2-(((2′-(trifluoromethyl)-[1,1′-biphenyl]-4-yl)oxy)methyl)morpholino)sulfonyl)-1H-indole-2-carboxamide (38)

Synthesized by similar route as represented in scheme-2 by using 2-trifluoromethylphenylboronic acid in step-2.

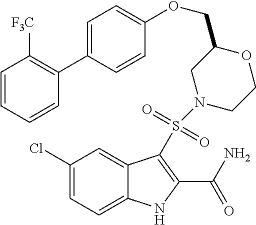

¹H NMR (CDCl₃, 300 MHz): δ 10.5 (s, 1H), 9.1 (s, 1H), 9.09 (s, 1H), 7.77 (d, J=13.5 Hz, 1H), 7.57-7.43 (m, 3H), 7.41 (d, J=9 Hz, 1H), 7.32 (d, J=7.5 Hz, 1H), 7.26 (m, 3H), 6.91 (d, J=8.7 Hz, 2H), 4.07-3.92 (m, 5H), 3.84-3.77 (m, 1H), 3.65 (d, J=11.7 Hz, 1H), 2.62 (t, J=3.3 Hz, 1H), 2.53 (t, J=11.1 Hz, 1H).

MS (m/z): 592 [M−H]

### Preparation of (S)-5-chloro-3-((2-(((4′-methoxy-[1,1′-biphenyl]-4-yl)oxy)methyl)morpholino)sulfonyl)-1H-indole-2-carboxamide (39)

Synthesized by similar route as represented in scheme-2 by using 4-methoxyphenylboronic acid in step-2.

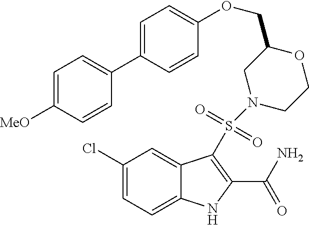

¹H NMR (CDCl₃, 300 MHz): δ 10.34 (s, 1H), 9.06 (s, 1H), 8.17 (s, 1H), 7.50-7.38 (m, 6H), 6.98-6.90 (m, 4H), 6.01 (s, 1H), 4.06-3.92 (m, 4H), 3.86 (s, 3H), 3.83-3.73 (m, 2H), 3.64-3.60 (d, J=11.1 Hz, 1H), 2.63-2.57 (m, 1H), 2.51-2.44 (m, 1H).

MS (m/z): 556 [M+H]

### Preparation of (S)-5-chloro-3-((2-(((2′-(trifluoromethoxy)-[1,1′-biphenyl]-4-yl)oxy)methyl)morpholino)sulfonyl)-1H-indole-2-carboxamide (40)

Synthesized by similar route as represented in scheme-2 by using (2-(trifluoromethoxy)phenyl)boronic acid in step-2.

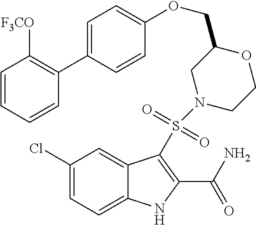

¹H NMR (CDCl₃, 300 MHz): δ 10.55 (s, 1H), 9.08 (s, 1H), 8.17 (s, 1H), 7.97-7.95 (d, J=6.9 Hz, 1H), 7.61-7.48 (m, 3H), 7.45-7.41 (m, 2H), 7.38 (s, 1H), 7.35-7.32 (d, J=3.6 Hz, 2H), 6.95-6.91 (dd, J=2.7 Hz, 2H), 4.07-3.92 (m, 4H), 3.84-3.73 (m, 2H), 3.65-3.61 (d, J=11.4 Hz, 1H), 2.65-2.58 (m, 1H), 2.53-2.46 (m, 1H).

MS (m/z): 610 [M+H]

### Preparation of (S)-5-chloro-3-((2-(((2′-methyl-[1,1′-biphenyl]-4-yl)oxy)methyl)morpholino)sulfonyl)-1H-indole-2-carboxamide (41)

Synthesized by similar route as represented in scheme-2 by using o-tolylboronic acid in step-2.

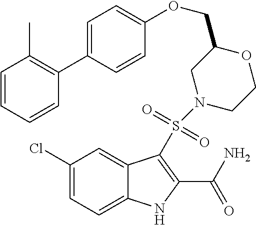

¹H NMR (CDCl₃, 300 MHz): δ 10.63 (s, 1H), 9.11 (s, 1H), 8.17 (s, 1H) 7.53 (d, J=9 Hz, 1H), 7.41 (dd, J=8.7 Hz, 1H), 7.27-7.20 (m, 6H), 6.92 (d, J=8.7 Hz, 2H), 6.13 (s, 1H), 4.03-3.91 (m, 4H), 3.85-3.73 (m, 2H), 3.65 (d, J=11.4 Hz, 1H), 2.65-2.46 (m, 2H), 2.27 (s, 3H).

MS (m/z): 540 [M+H]

### Preparation of (S)-5-chloro-3-((2-(((2′-fluoro-[1,1′-biphenyl]-4-yl)oxy)methyl)morpholino)sulfonyl)-1H-indole-2-carboxamide (42)

Synthesized by similar route as represented in scheme-2 by using 2-fluorophenylboronic acid in step-2.

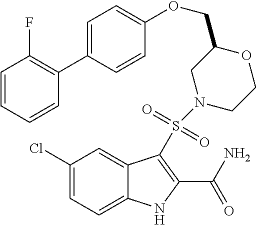

¹H NMR (DMSO-d₆, 300 MHz): δ 13.04 (s, 1H), 8.25 (d, J=12 Hz, 2H), 7.9 (d, J=3 Hz, 1H), 7.58-7.52 (m, 2H), 7.38-7.30 (m, 3H), 7.31-7.26 (m, 2H), 7.12 (d, J=9 Hz, 1H), 7.03 (s, 1H), 6.90-6.88 (d, J=9 Hz, 1H), 4.03 (d, J=6 Hz, 2H), 3.80 (d, 1H), 3.85-3.83 (m, 1H), 3.56 (d, J=6 Hz, 1H), 3.50 (m, 2H), 2.4 (m, 2H).

MS: (m/z) 544 [M+H]

### Preparation of (S)-5-chloro-3-((2-(((2′-methoxy-[1,1′-biphenyl]-4-yl)oxy)methyl)morpholino)sulfonyl)-1H-indole-2-carboxamide (43)

Synthesized by similar route as represented in scheme-2 by using o-methoxyphenylboronic acid in step-2.

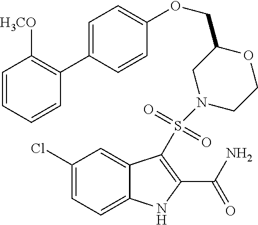

¹H NMR (DMSO-d₆, 300 MHz): δ 13.01 (s, 1H), 8.22 (d, J=13.8 Hz, 2H), 7.87 (d, J=13.8 Hz, 1H), 7.56 (d, J=9 Hz, 1H), 7.34 (d, J=9 and 2.1 Hz, 2H), 7.28-7.22 (m 2H), 7.08 (d, J=8.1 Hz, 1H), 7.01 (t, J=7.5 Hz, 2H), 6.93 (s, 1H), 6.84-6.82 (m, 1H), 3.99-3.96 (m, 2H), 3.92 (d, J=14 and 12 Hz, 1H), 3.88-3.82 (m, 1H), 3.7 (s, 3H), 3.6 (s, 1H), 3.57-3.48 (m, 2H), 2.47-2.44 (m, 2H).

MS: (m/z) 556 [M+H]

### Preparation of (S)-5-chloro-3-((2-((4-(2-methoxypyridin-3-yl)phenoxy)methyl)morpholino)sulfonyl)-1H-indole-2-carboxamide (44)

Synthesized by similar route as represented in scheme-2 by using 2-methoxypyridine-3-boronic acid in step-2.

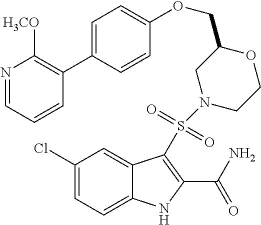

¹H NMR (CDCl₃, 300 MHz): δ 10.10 (br. s, 1H), 9.00 (br. s, 1H), 8.16 (d, J=1.8 Hz, 1H), 8.12 (m, 1H), 7.59 (dd, J=7.2 and 1.8 Hz, 1H), 7.45 (d, J=8.7 Hz, 1H), 7.37 (dd, J=9.0 and 1.8 Hz, 1H), 7.31 (d, J=8.1 Hz, 1H), 7.15 (d, J=7.5 Hz, 1H), 7.04 (m, 1H), 6.98 (dd, J=7.5 and 5.1 Hz, 1H), 6.85 (dd, J=6.3 and 1.8 Hz, 1H), 5.90 (br. s, 1H), 4.04-3.91 (m, 7H), 3.80-3.71 (m, 2H), 3.61 (d, J=12.6 Hz, 1H), 2.58 (dt, J=8.1 and 3.6 Hz, 1H), 2.46 (t, J=9.9 Hz, 1H).

MS: (m/z) 557 [M+H]

### Preparation of (S)-5-chloro-3-((2-((4-(2-fluoropyridin-4-yl)phenoxy)methyl)morpholino)sulfonyl)-1H-indole-2-carboxamide (45)

Synthesized by similar route as represented in scheme-2 by using (2-fluoropyridin-4-yl)boronic acid step-2.

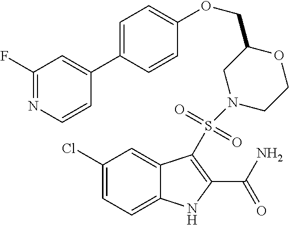

¹H NMR (DMSO-d₆, 300 MHz): δ 12.96 (s, 1H), 8.22 (d, J=5.7 Hz, 3H), 7.88 (s, 1H), 7.80 (d, J=8.7 Hz, 2H), 7.64 (d, J=4.8 Hz, 1H), 7.56 (d, J=8.7 Hz, 1H), 7.45 (s, 1H), 7.35 (d, J=8.7 Hz, 1H), 7.03 (d, J=8.4 Hz, 2H), 4.04 (d, J=4.5 Hz, 2H), 3.92-3.68 (m, 3H), 3.53 (m, 2H), 2.47-2.37 (m, 2H).

MS (m/z): 545 [M+H]

### Preparation of (S)-5-chloro-3-((2-((4-(6-fluoropyridin-3-yl)phenoxy)methyl)morpholino)sulfonyl)-1H-indole-2-carboxamide (46)

Synthesized by similar route as represented in scheme-2 by using (6-fluoropyridin-3-yl)boronic acid in step-2.

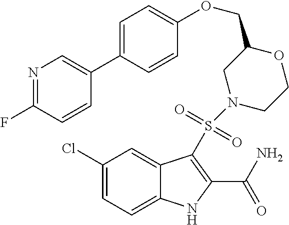

¹H NMR (DMSO-d₆, 300 MHz): δ 8.46 (s, 1H), 8.22 (t, J=5.7 Hz, 1H), 8.05 (s, 1H), 7.89 (s, 1H), 7.84 (s, 1H), 7.61 (d, J=8.4 Hz, 2H), 7.52 (d, J=8.7 Hz, 1H), 7.23 (m, 2H), 7.01 (d, J=8.4 Hz, 2H), 4.00 (d, J=4.8 Hz, 2H), 3.90-3.68 (m, 3H), 3.56-3.49 (m, 2H), 2.47-2.32 (m, 2H).

MS (m/z): 545 [M+H]

### Preparation of (S)-5-chloro-3-((2-((4-(2-methoxypyridin-4-yl)phenoxy)methyl)morpholino)sulfonyl)-1H-indole-2-carboxamide (47)

Synthesized by similar route as represented in scheme-2 by using (2-methoxypyridin-4-yl)boronic acid in step-2.

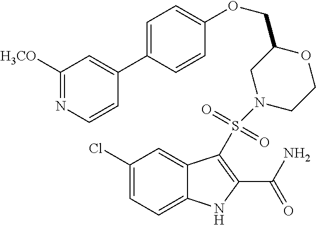

¹H NMR (DMSO-d₆, 300 MHz): δ 8.18-8.11 (m, 3H), 7.86 (s, 1H), 7.70 (d, J=8.7 Hz, 2H), 7.55 (d, J=8.7 Hz, 1H), 7.32 (d, J=7.2 Hz, 1H), 7.25 (d, J=4.8 Hz, 1H), 7.02 (m, J=6.9, 8.7 Hz, 1H), 4.02 (d, J=4.5 Hz, 2H), 3.91-3.80 (m, 2H), 3.85 (s, 3H), 3.72 (d, J=11.7 Hz, 2H), 3.57 (t, J=12 Hz, 2H), 2.04-2.33 (m, 2H). MS (m/z): 558 [M+2H]

### Preparation of (S)-5-chloro-3-((2-((4-(pyrimidin-5-yl)phenoxy)methyl)morpholino)sulfonyl)-1H-indole-2-carboxamide (48)

Synthesized by similar route as represented in scheme-2 by using pyrimidin-5-ylboronic acid in step-2.

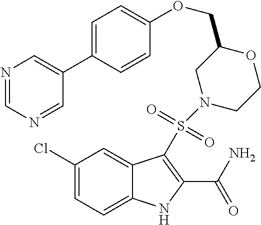

¹H NMR (CDCl₃, 300 MHz): δ 9.15 (s, 1H), 9.02 (s, 1H), 8.90 (s, 2H), 8.14 (s, 1H), 7.50-7.47 (d, J=8.7 Hz, 3H), 7.38-7.35 (d, J=10.5 Hz, 1H), 7.00-6.97 (d, J=8.4 Hz, 1H), 6.06 (s, 1H), 4.06-3.94 (m, 4H), 3.80-3.72 (m, 2H), 3.62-3.58 (d, J=12.3 Hz, 1H), 2.62-2.54 (t, J=10.8 Hz, 1H).

MS (m/z): 528 [M+H]

### Preparation of (S)-5-chloro-3-((2-((4-(2-fluoropyridin-3-yl)phenoxy)methyl)morpholino)sulfonyl)-1H-indole-2-carboxamide (49)

Synthesized by similar route as represented in scheme-2 by using (2-fluoropyridin-3-yl)boronic acid in step-2.

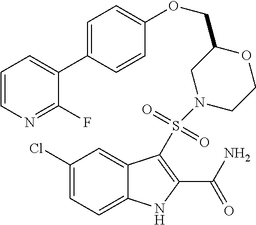

¹H NMR (CDCl₃, 300 MHz): δ 8.14 (s, 2H), 7.95-7.92 (d, J=7.2 Hz, 1H), 7.85-7.80 (t, J=9 Hz, 1H), 7.62-7.47 (m, 5H), 7.39-7.36 (d, J=9 Hz, 1H), 6.95-6.93 (d, J=8.7 Hz, 1H), 4.91 (s, 1H), 4.06-3.91 (m, 4H), 3.81-3.72 (m, 2H), 3.63-3.59 (d, J=12.3 Hz, 1H), 2.63-2.54 (t, J=11.1 Hz, 1H), 2.50-2.43 (t, J=10.2 Hz, 1H).

MS (m/z): 545 [M+H]

### Preparation of (S)-5-chloro-3-((2-((4-(2-methylpyridin-3-yl)phenoxy)methyl)morpholino)sulfonyl)-1H-indole-2-carboxamide (50)

Synthesized by similar route as represented in scheme-2 by using (2-methylpyridin-3-yl)boronic acid in step-2.

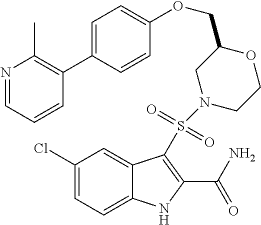

¹H NMR (CDCl₃, 300 MHz): δ 9.07 (s, 1H), 8.47-8.46 (d, J=3.6 Hz, 1H), 8.15 (s, 1H), 7.56-7.47 (m, 2H), 7.39-7.36 (dd, J=9 Hz, 1H), 7.26-7.14 (m, 3H), 6.94-6.91 (d, J=9 Hz, 2H), 4.04-3.91 (m, 4H), 3.83-3.72 (m, 2H), 3.63-3.59 (d, J=11.4 Hz, 1H), 2.63-2.55 (t, J=11.4 Hz, 1H), 2.49 (s, 3H), 2.47-2.43 (m, 1H).

MS (m/z): 541 [M+H]

### Preparation of (S)-5-chloro-3-((2-((4-(2-methylpyridin-4-yl)phenoxy)methyl)morpholino)sulfonyl)-1H-indole-2-carboxamide (51)

Synthesized by similar route as represented in scheme-2 by using (2-methylpyridin-4-yl)boronic acid in step-2.

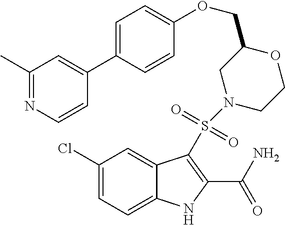

¹H NMR (CDCl₃, 300 MHz): δ 8.42-8.40 (d, J=5.4 Hz, 1H), 8.24-8.20 (d, J=13.5 Hz, 2H), 7.89-7.88 (d, J=1.5 Hz, 1H), 7.71-7.68 (d, J=8.7 Hz, 2H), 7.57-7.51 (m, J=8.7 Hz, 2H), 7.43-7.41 (d, J=5.1 Hz, 1H), 7.37-7.33 (dd, J=8.7 Hz, 1H), 7.01-6.98 (d, J=8.7 Hz, 2H), 4.03-4.01 (d, J=4.8 Hz, 2H), 3.89-3.68 (m, 3H), 3.54-3.50 (d, J=12.9 Hz, 2H), 2.48 (s, 3H), 2.41-2.34 (t, J=10.5 Hz, 2H).

MS (m/z): 541 [M+H]

### Preparation of (S)-5-chloro-3-((2-((4-(6-methylpyridin-3-yl)phenoxy)methyl)morpholino)sulfonyl)-1H-indole-2-carboxamide (52)

Synthesized by similar route as represented in scheme-2 by using (2-methylpyridin-5-yl)boronic acid in step-2.

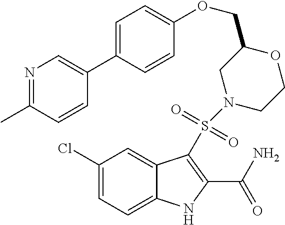

¹H NMR (DMSO-d₆, 300 MHz): δ 9.06 (s, 1H), 8.67-8.66 (d, J=2.1 Hz, 1H), 8.15 (s, 1H), 7.72-7.69 (dd, J=8.1 Hz, 1H), 7.49-7.44 (m, 3H), 7.38-7.35 (dd, J=8.7 Hz, 1H), 7.20-7.18 (d, J=7.8 Hz, 1H), 6.94-6.92 (d, J=8.7 Hz, 2H), 4.05-3.90 (m, 4H), 3.81-3.71 (m, 2H), 3.63-3.59 (d, J=11.7 Hz, 1H), 2.58 (s, 3H), 2.63-2.54 (m, 1H), 2.49-2.42 (t, J=11.1 Hz, 1H).

MS (m/z): 541 [M+H]

### Preparation of (S)-5-chloro-3-((2-((4-(pyrimidin-2-yl)phenoxy)methyl)morpholino)sulfonyl)-1H-indole-2-carboxamide (53)

Synthesized by similar route as represented in scheme-2 by using pyrimidin-2-ylboronic acid in step-2.

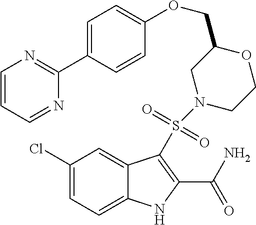

¹H NMR (CDCl₃, 300 MHz): δ 12.25 (br. s, 1H), 8.70 (br. s, 1H), 8.55 (d, J=3.0 Hz, 1H), 8.67 (d, J=2.7 Hz, 1H), 8.28 (d, J=5.1 Hz, 1H), 8.01 (s, 1H), 7.50 (d, J=5.4 Hz, 1H), 7.22 (d, J=5.4 Hz, 1H), 7.07 (m, 1H), 6.87 (d, J=5.4 Hz, 1H), 6.51 (br. s, 1H), 3.97-3.87 (m, 4H), 3.68-3.63 (m, 2H), 3.50 (d, J=11.1 Hz, 1H), 2.42 (dt, J=11.4 and 3.0 Hz, 1H), 2.37 (t, J=9.9 Hz, 1H).

MS: (m/z) 528 [M+H]

### Preparation of (S)-3-((2-(([1,1′-biphenyl]-2-yloxy)methyl)morpholino)sulfonyl)-5-chloro-1H-indole-2-carboxamide (54)

Synthesized by similar route as represented in scheme-2 by using 2-bromophenol in step-1 and phenylboronic acid in step-2.

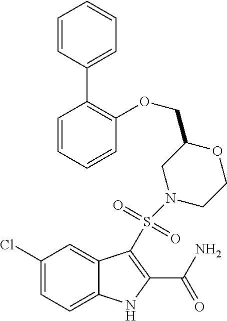

¹H NMR (DMSO-d₆, 300 MHz): δ 13.05 (s, 1H), 8.25 (m, 2H), 7.89 (d, J=2.1 Hz, 1H), 7.6 (d, J=9.0 Hz, 1H), 7.38 (m, 3H), 7.29 (m, 2H), 7.19 (d, J=3.0 Hz, 3H), 7.04 (d, J=7.5 Hz, 2H), 4.07 (dd, J=10.2 and 4.5 Hz, 1H), 3.98 (dd, J=10.2 and 4.8 Hz, 1H), 3.87 (m, 1H), 3.76 (m, 1H), 3.69 (d, J=11.1 Hz, 1H), 3.52 (t, J=11.4 Hz, 2H), 2.35 (t, J=10.5 Hz, 2H).

MS: (m/z) 526 [M+H], 391, 149.

### Preparation of (S)-5-chloro-3-((2-((2-(pyridin-3-yl)phenoxy)methyl)morpholino)sulfonyl)-1H-indole-2-carboxamide (55)

Synthesized by similar route as represented in scheme-2 by using 2-bromophenol in step-1 and pyridin-3-ylboronic acid in step-2.

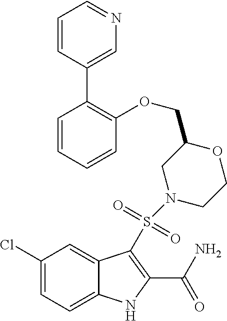

¹H NMR (DMSO-d₆, 300 MHz): δ 13.05 (s, 1H), 8.60 (d, J=1.8 Hz, 1H), 8.4 (m, 1H) 8.25 (m, 2H), 7.87 (d, J=1.8 Hz, 1H), 7.82 (m, 1H), 7.61 (t, J=8.7 Hz, 2H), 7.34 (m, 2H), 7.16 (dd, J=7.8 and 4.8 Hz, 1H), 7.07 (m, 2H), 4.06 (m, 2H) 3.92 (d, J=2.1 Hz, 1H), 3.77 (m, 1H), 3.64 (d, J=10.8 Hz, 1H), 3.52 (t, J=11.4 Hz, 2H), 2.36 (m, 2H).

MS: (m/z) 527 [M+H]

### Preparation of (S)-5-chloro-3-((2-((2-(pyridin-4-yl)phenoxy)methyl)morpholino)sulfonyl)-1H-indole-2-carboxamide (56)

Synthesized by similar route as represented in scheme-2 by using 2-bromophenol in step-1 and pyridin-4-ylboronic acid in step-2.

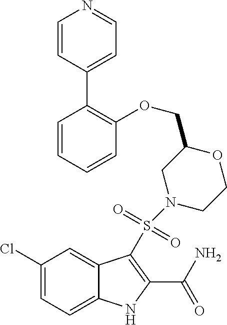

¹H NMR (DMSO-d₆, 300 MHz): δ 13.07 (s, 1H), 8.38 (d, J=5.7 Hz, 2H), 8.27 (m, 2H), 7.87 (m, J=1.5 Hz, 1H), 7.61 (d, J=8.7 Hz, 1H), 7.38 (m, 5H), 7.08 (m, 2H), 4.08 (m, 1H), 4.00 (dd, J=10.2 and 4.5 Hz, 1H), 3.92 (d, J=11.4 Hz, 1H), 3.77 (m, 1H), 3.7 (d, J=11.4 Hz, 1H), 3.52 (t, J=11.7 Hz, 2H), 2.38 (dd, J=21.6 and 10.8 Hz, 2H).

MS: (m/z) 527 [M+H]

### Preparation of (S)-5-chloro-3-((2-((2-(2-fluoropyridin-3-yl)phenoxy)methyl)morpholino)sulfonyl)-1H-indole-2-carboxamide (57)

Synthesized by similar route as represented in scheme-2 by using 2-bromophenol in step-1 and (2-fluoropyridin-3-yl)boronic acid in step-2.

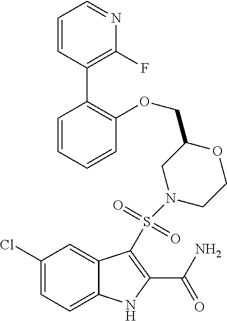

¹H NMR (DMSO-d₆, 300 MHz): δ 13.03 (s, 1H), 8.24 (d, J=17.7 Hz, 2H), 8.10 (d, J=4.2 Hz, 1H), 7.86 (m, 2H), 7.61 (d, J=8.7 Hz, 1H), 7.38 (m, 2H), 7.30 (d, J=7.5 Hz, 1H), 7.19 (m, 1H), 7.07 (m, 2H), 4.04 (m, 2H), 3.84 (m, 1H), 3.70 (m, 1H), 3.58 (m, 1H), 3.45 (m, 2H), 3.36 (m, 1H), 2.2 (m, 1H).

MS: (m/z) 545 [M+H]

### Preparation of (S)-5-chloro-3-((2-(((2′-methoxy-[1,1′-biphenyl]-2-yl)oxy)methyl)morpholino)sulfonyl)-1H-indole-2-carboxamide (58)

Synthesized by similar route as represented in scheme-2 by using 2-bromophenol in step-1 and 2-methoxyphenylboronic acid in step-2.

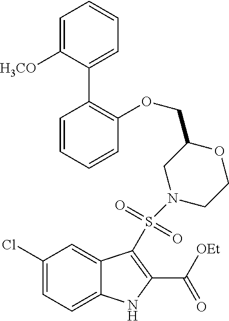

¹H NMR (DMSO-d₆, 300 MHz): δ 13.04 (s, 1H), 8.23 (d, J=12.6 Hz, 2H), 7.86 (d, J=1.8 Hz, 1H), 7.62 (t, J=9.0 Hz, 1H), 7.40 (dd, J=8.7 and 2.1 Hz, 1H), 7.25 (m, 1H) 7.13 (m, 2H), 7.05 (dd, J=7.5 and 1.5, 1H), 6.95 (dd, J=13.8 and 7.5 Hz, 2H), 6.88 (d, J=8.4 Hz, 1H), 6.78 (m, 1H), 3.98-3.82 (m, 3H), 3.60 (s, 3H), 3.47 (m, 2H), 2.3 (m, 1H), 2.2 (t, J=10.8 Hz, 1H).

MS: (m/z) 556 [M+H]

### Preparation of (S)-5-chloro-3-((2-((2-(6-fluoropyridin-3-yl)phenoxy)methyl)morpholino)sulfonyl)-1H-indole-2-carboxamide (59)

Synthesized by similar route as represented in scheme-2 by using 2-bromophenol in step-1 and (2-fluoropyridin-5-yl)boronic acid in step-2.

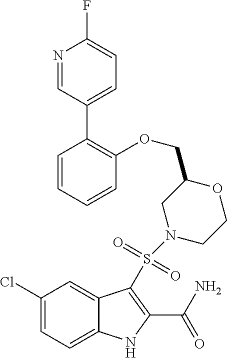

¹H NMR (DMSO-d₆, 300 MHz): δ 13.01 (s, 1H), 8.26 (br. s, 2H), 8.18 (br. s, 1H), 8.04 (d, J=2.4 Hz, 1H), 7.85 (d, J=1.8 Hz, 1H), 7.60 (d, J=9 Hz, 1H), 7.35 (m, 3H), 7.08 (dd, J=15.6 and 8.4 Hz, 2H), 6.93 (dd, J=8.4 and 2.7 Hz, 1H), 4.07 (m, 2H), 3.88 (d, 1H), 3.8 (m, 1H), 3.66 (d, J=11.1 Hz, 1H), 3.52 (d, J=12 Hz, 2H), 2.36 (m, 2H).

MS: (m/z) 545 [M+H]

### Preparation of (S)-5-chloro-3-((2-((2-(pyrimidin-5-yl)phenoxy)methyl)morpholino)sulfonyl)-1H-indole-2-carboxamide (60)

Synthesized by similar route as represented in scheme-2 by using 2-bromophenol in step-1 and pyrimidin-5-ylboronic acid in step-2.

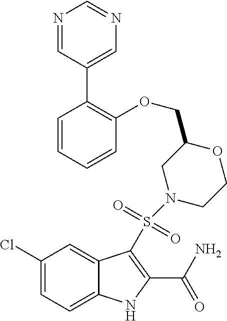

¹H NMR (DMSO-d₆, 300 MHz): δ 12.99 (s, 1H), 8.99 (s, 1H), 8.88 (s, 2H), 8.23 (d, J=21.9 Hz, 2H), 7.87 (d, J=1.8 Hz, 1H), 7.60 (d, J=8.7 Hz, 1H), 7.46 (m, 2H), 7.36 (dd, J=8.7 and 1.8 Hz, 1H), 7.12 (dd, J=15.9 and 8.4 Hz, 2H), 4.09 (d, J=4.5 Hz, 2H), 3.94 (d, J=11.4 Hz, 1H), 3.8 (m, 1H), 3.62 (d, J=11.4 Hz, 1H), 3.52 (m, 2H), 2.45 (m, 1H), 2.30 (t, J=10.8 Hz, 1H).

MS: (m/z) 526 [M−H]

### Preparation of (S)-5-chloro-3-((2-((2-(6-methoxypyridin-3-yl)phenoxy)methyl)morpholino)sulfonyl)-1H-indole-2-carboxamide (61)

Synthesized by similar route as represented in scheme-2 by using 2-bromophenol in step-1 and (2-methoxypyridin-5-yl)boronic acid in step-2.

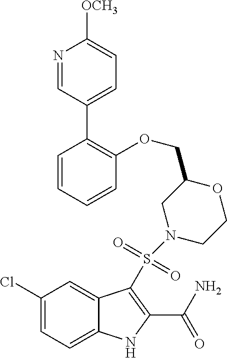

¹H NMR (DMSO-d₆, 300 MHz): δ 13.01 (s, 1H), 8.24 (br. s, 1H), 8.17 (d, J=2.4 Hz, 2H), 7.88 (d, J=1.8 Hz, 1H), 7.74 (dd, J=8.7 and 2.4 Hz, 1H), 7.50 (d, J=8.7 Hz, 1H), 7.31 (m, 3H), 7.01 (m, 2H), 6.58 (d, J=8.4 Hz, 1H), 4.02 (m, 1H), 3.95 (m, 1H), 3.85 (br. s, 1H), 3.82 (s, 3H), 3.76 (m, 1H), 3.68 (d, J=11.1 Hz, 1H), 3.49 (t, J=11.7 Hz, 2H), 2.36 (m, 2H).

MS: (m/z) 557 [M+H]

### Preparation of (S)-5-chloro-3-((2-(((2′-fluoro-[1,1′-biphenyl]-2-yl)oxy)methyl)morpholino)sulfonyl)-1H-indole-2-carboxamide (62)

Synthesized by similar route as represented in scheme-2 by using 2-bromophenol in step-1 and 2-fluorophenylboronic acid in step-2.

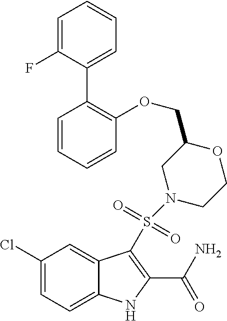

¹H NMR (DMSO-d₆, 300 MHz): δ 13.03 (s, 1H), 8.22 (d, J=15.3 Hz, 1H), 7.87 (d, J=1.8 Hz, 1H), 7.62 (dd, J=12.3 and 8.7 Hz, 1H), 7.37 (m, 2H), 7.24 (m, 4H) 7.01 (m, 4H), 4.04 (dd, J=10.2 and 3.9 Hz, 1H) 3.92 (dd, J=9.9 and 4.8 Hz, 1H), 3.84 (br. s, 1H), 3.65 (s, 1H), 3.55 (m, 1H), 3.43 (m, 2H), 2.27 (m, 2H).

MS: (m/z) 542 [M−H]

### Preparation of (S)-5-chloro-3-((2-((2-(2-methylpyridin-3-yl)phenoxy)methyl)morpholino)sulfonyl)-1H-indole-2-carboxamide (63)

Synthesized by similar route as represented in scheme-2 by using 2-bromophenol in step-1 and (2-methylpyridin-3-yl)boronic acid in step-2.

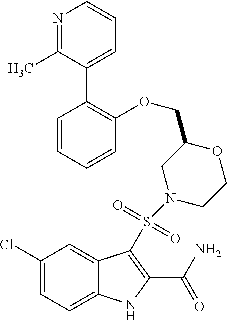

¹H NMR (DMSO-d₆, 300 MHz): δ 13.01 (s, 1H), 8.20 (d, J=15.3 Hz, 3H), 7.81 (d, J=1.5 Hz, 1H), 7.60 (d, J=8.7 Hz, 1H), 7.34 (m, 3H), 7.02 (m, 4H), 3.97 (m, 2H), 3.79 (br. m, 1H), 3.60 (m, 1H), 3.44 (t, J=10.5 Hz, 3H), 3.15 (d, J=4.8 Hz, 1H), 2.15 (s, 3H), 2.08 (m, 1H).

MS: (m/z) 541 [M+H]

### Preparation of (S)-5-chloro-3-((2-(((3′-fluoro-[1,1′-biphenyl]-2-yl)oxy)methyl)morpholino)sulfonyl)-1H-indole-2-carboxamide (64)

Synthesized by similar route as represented in scheme-2 by using 2-bromophenol in step-1 and 3-fluorophenylboronic acid in step-2.

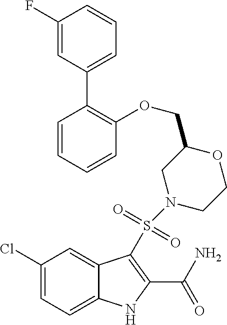

¹H NMR (DMSO-d₆, 300 MHz): δ 12.99 (s, 1H), 8.22 (d, J=17.7 Hz, 2H), 7.84 (d, J=1.8 Hz, 1H), 7.58 (m, 1H), 7.34 (m, 2H), 7.28 (m, 2H), 7.21 (m, 2H), 7.01 (m, 3H), 4.04 (m, 1H), 3.92 (d, J=11.1 Hz, 1H), 3.76 (m, 1H), 3.64 (d, J=10.8 Hz, 1H), 3.51 (m, 2H), 3.39 (t, J=6.9 Hz, 1H), 2.37 (m, 2H).

MS: (m/z) 544 [M+H]

### Preparation of (S)-5-chloro-3-((2-(((2′-fluoro-4′-(trifluoromethyl)-[1,1′-biphenyl]-2-yl)oxy)methyl)morpholino)sulfonyl)-1H-indole-2-carboxamide (65)

Synthesized by similar route as represented in scheme-2 by using 2-bromophenol in step-1 and (2-fluoro-5-(trifluoromethyl)phenyl)boronic acid in step-2.

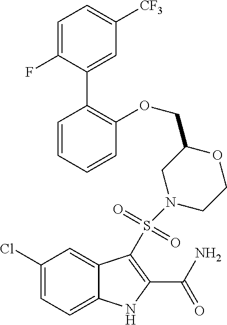

¹H NMR (DMSO-d₆, 300 MHz): δ 12.99 (s, 1H), 8.21 (d, J=12 Hz, 2H), 7.84 (d, J=1.5 Hz, 1H), 7.64 (m, 1H), 7.60 (m, 2H), 7.37 (m, 2H), 7.27 (dd, J=17.1 and 7.5 Hz, 2H), 7.06 (dd, J=18.3 and 8.7 Hz, 2H), 4.04 (m, 2H), 3.82 (d, J=11.4 Hz, 1H), 3.66 (m, 1H), 3.56 (d, J=11.4 Hz, 1H), 3.45 (m, 2H), 2.25 (m, 2H).

MS: (m/z) 612 [M+H]

### Preparation of (S)-5-chloro-3-((2-((3-methyl-4-(pyridin-3-yl)phenoxy)methyl)morpholino)sulfonyl)-1H-indole-2-carboxamide (66)

Synthesized by similar route as represented in scheme-2 by using 4-bromo-3-methylphenol in step-1 and 3-pyridinylboronic acid in step-2.

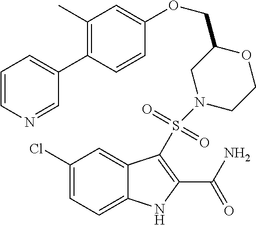

¹H NMR (DMSO-d₆, 300 MHz): δ 13.01 (s, 1H), 8.51 (m, 2H), 8.23 (d, J=13.5 Hz, 2H) 7.84 (d, J=1.8 Hz, 1H), 7.73 (m, 1H), 7.57 (d, J=8.7 Hz, 1H), 7.43 (q, J=4.8 and 7.8 Hz, 1H), 7.36 (dd, J=2.1 and 8.7 Hz, 1H), 7.12 (d, J=8.4 Hz, 1H), 6.86 (ddd, J=2.1, 2.4 and 2.7 Hz, 2H) 3.99 (d, J=4.2 Hz, 2H), 3.92 (d, J=11.4 Hz, 1H), 3.81 (m, 1H), 3.71 (d, J=11.4 Hz, 1H), 3.54 (m, 2H), 2.41 (t, J=10.8 Hz, 2H), 2.17 (s, 3H) MS: (m/z) 541 [M+H]

### Preparation of (S)-5-chloro-3-((2-((3-methyl-4-(pyridin-4-yl)phenoxy)methyl)morpholino)sulfonyl)-1H-indole-2-carboxamide (67)

Synthesized by similar route as represented in scheme-2 by using 4-bromo-3-methylphenol in step-1 and 4-pyridinylboronic acid in step-2.

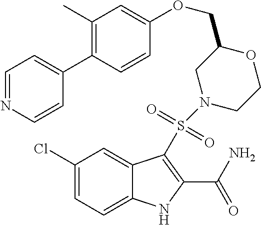

¹H NMR (DMSO-d₆, 300 MHz): δ 13.02 (s, 1H), 8.51 (d, J=5.1 Hz, 2H), 8.24 (d, J=12.3 Hz, 2H), 7.88 (d, J=1.5 Hz, 1H), 7.57 (d, J=9.0 Hz, 1H), 7.36 (dd, J=6.0 and 1.8 Hz, 3H), 7.14 (d, J=8.4 Hz, 1H), 6.82 (m, 2H), 3.99 (d, J=4.8 Hz, 2H), 3.92 (d, J=10.8 Hz, 1H), 3.82 (m, 1H), 3.71 (d, J=11.1 Hz, 1H), 3.54 (m, 2H), 2.39 (t, J=10.8 and 11.1 Hz, 2H), 2.20 (s, 3H).

MS: (m/z) 541 [M+H]

### Preparation of (S)-5-chloro-3-((2-((4-(2-fluoropyridin-3-yl)-3-methylphenoxy)methyl)morpholino)sulfonyl)-1H-indole-2-carboxamide (68)

Synthesized by similar route as represented in scheme-2 by using 4-bromo-3-methylphenol in step-1 and (2-fluoropyridin-3-yl)boronic acid in step-2.

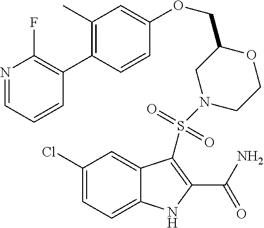 [SMILES: Cc1cc(OC[C@@H]2CN(S(=O)(=O)c3c(C(N)=O)[nH]c4ccc(Cl)cc34)CCO2)ccc1-c1cccnc1F]

¹H NMR (DMSO-d₆, 300 MHz): δ 13.02 (s, 1H), 8.23 (t, J=6.6 and 4.5 Hz, 3H), 7.88 (d, J=1.8 Hz, 1H), 7.85 (m, 1H), 7.57 (d, J=8.7 Hz, 1H), 7.4 (m, 1H), 7.36 (dd, J=8.7 and 2.1, 1H), 7.12 (d, J=8.4 Hz, 1H), 6.87 (d, J=2.1 Hz, 1H), 6.8 (m, 1H), 3.99 (d, J=4.8 Hz, 2H), 3.92 (d, J=11.7 Hz, 1H), 3.83 (m, 1H), 3.71 (d, J=11.4 Hz, 1H), 3.54 (m, 2H), 2.41 (t, J=11.1 and 10.8 Hz, 2H), 2.06 (s, 3H).

MS: (m/z) 557 [M−H]

### Preparation of (S)-5-chloro-3-((2-((4-(6-methoxypyridin-3-yl)-3-methylphenoxy)methyl)morpholino)sulfonyl)-1H-indole-2-carboxamide (69)

Synthesized by similar route as represented in scheme-2 by using 4-bromo-3-methylphenol in step-1 and (2-methoxypyridin-5-yl)boronic acid in step-2.

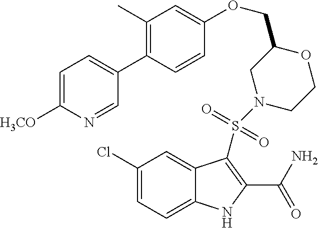 [SMILES: COc1ccc(-c2ccc(OC[C@@H]3CN(S(=O)(=O)c4c(C(N)=O)[nH]c5ccc(Cl)cc45)CCO3)cc2C)cn1]

¹H NMR (DMSO-d₆, 300 MHz): δ 13.02 (s, 1H), 8.24 (d, J=12.0 Hz, 2H), 8.05 (d, J=2.4 Hz, 1H), 7.88 (d, J=1.8 Hz, 1H), 7.65 (dd, J=8.4 and 2.4 Hz, 1H), 7.57 (d, J=9.0 Hz, 1H), 7.36 (dd, J=8.7 and 1.8 Hz, 1H), 7.08 (d, J=8.4 Hz, 1H), 6.84 (m, 2H), 6.78 (d, J=2.4 Hz, 1H), 3.97 (d, J=3.9 Hz, 2H), 3.92 (d, J=12.0 Hz, 1H), 3.85 (s, 3H), 3.8 (m, 1H), 3.71 (d, J=11.1 Hz, 1H), 3.54 (m, 2H), 2.44 (t, J=12.9 and 10.8 Hz, 2H), 2.15 (s, 3H).

MS: (m/z) 571 [M+H]

### Preparation of (S)-5-chloro-3-((2-((4-(2-methoxypyridin-4-yl)-3-methylphenoxy)methyl)morpholino)sulfonyl)-1H-indole-2-carboxamide (70)

Synthesized by similar route as represented in scheme-2 by using 4-bromo-3-methylphenol in step-1 and (2-methoxypyridin-4-yl)boronic acid in step-2.

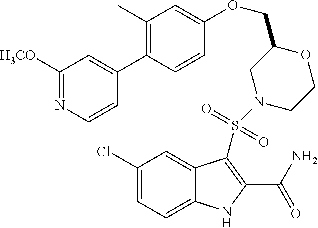 [SMILES: COc1cc(-c2ccc(OC[C@@H]3CN(S(=O)(=O)c4c(C(N)=O)[nH]c5ccc(Cl)cc45)CCO3)cc2C)ccn1]

¹H NMR (DMSO-d₆, 300 MHz): δ 13.02 (s, 1H), 8.23 (d, J=12.6 Hz, 2H), 8.15 (d, J=5.4 Hz, 1H), 7.88 (d, J=1.5 Hz, 1H), 7.57 (d, J=9.0 Hz, 1H), 7.36 (dd, J=1.8 Hz, 1H), 7.12 (d, J=8.4 Hz, 1H), 6.92 (m, 1H), 6.83 (br. s, 1H), 6.80 (dd, J=7.8 and 1.8 Hz, 1H), 6.69 (s, 1H), 3.98 (d, J=4.5 Hz, 2H), 3.92 (m, 1H), 3.84 (s, 3H), 3.81 (m, 1H), 3.71 (m, 1H), 3.54 (m, 2H), 2.39 (m, 2H), 2.19 (s, 3H).

MS: (m/z) 571 [M+H]

### Preparation of (S)-5-chloro-3-((2-((3-methyl-4-(pyrimidin-5-yl)phenoxy)methyl)morpholino)sulfonyl)-1H-indole-2-carboxamide (71)

Synthesized by similar route as represented in scheme-2 by using 4-bromo-3-methylphenol in step-1 and pyrimidin-5-ylboronic acid in step-2.

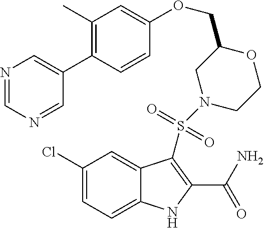 [SMILES: Cc1cc(OC[C@@H]2CN(S(=O)(=O)c3c(C(N)=O)[nH]c4ccc(Cl)cc34)CCO2)ccc1-c1cncnc1]

¹H NMR (DMSO-d₆, 300 MHz): δ 13.02 (s, 1H), 9.13 (s, 1H), 8.78 (s, 2H), 8.23 (d, J=12.3 Hz, 2H), 7.88 (d, J=1.8 Hz, 1H), 7.57 (d, J=8.7, Hz, 1H), 7.36 (dd, J=8.7 and 1.8 Hz, 1H), 7.20 (d, J=8.4 Hz, 1H), 6.89 (m, 1H), 6.82 (m, 1H), 4.00 (d, J=4.8 Hz, 2H), 3.92 (d, J=11.1 Hz, 1H), 3.80 (m, 1H), 3.71 (d, J=11.1 Hz, 1H), 3.54 (m, 2H), 2.40 (t, J=10.8 Hz, 2H), 2.20 (s, 3H).

MS: (m/z) 542 [M+H]

### Preparation of (S)-5-chloro-3-((2-((4-(2-fluoropyridin-4-yl)-3-methylphenoxy)methyl)morpholino)sulfonyl)-1H-indole-2-carboxamide (72)

Synthesized by similar route as represented in scheme-2 by using 4-bromo-3-methylphenol in step-1 and (2-fluoropyridin-4-yl)boronic acid in step-2.

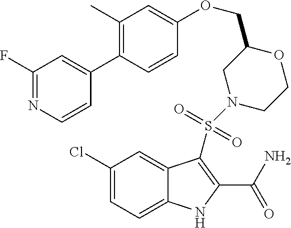 [SMILES: Cc1cc(OC[C@@H]2CN(S(=O)(=O)c3c(C(N)=O)[nH]c4ccc(Cl)cc34)CCO2)ccc1-c1ccnc(F)c1]

¹H NMR (DMSO-d₆, 300 MHz): δ 13.02 (s, 1H), 8.23 (d, J=12.9 Hz, 2H), 8.19 (s, 1H), 7.88 (d, J=1.8 Hz, 1H), 7.57 (d, J=9.0 Hz, 1H), 7.36 (dd, J=8.7 and 2.1 Hz, 1H), 7.31 (m, 1H), 7.19 (d, J=11.4 Hz, 1H), 7.13 (s, 1H), 6.86 (br. s, 1H), 6.80 (dd, J=10.8 and 2.4 Hz, 1H), 3.98 (d, J=4.8 Hz, 2H), 3.92 (m, 1H), 3.82 (s, 1H), 3.71 (d, J=11.1 Hz, 1H), 3.54 (m, 2H), 2.40 (t, J=10.8 Hz, 2H), 2.22 (s, 3H).

MS: (m/z) 559 [M+H]

### Preparation of (S)-5-chloro-3-((2-((4-(6-fluoropyridin-3-yl)-3-methylphenoxy)methyl)morpholino)sulfonyl)-1H-indole-2-carboxamide (73)

Synthesized by similar route as represented in scheme-2 by using 4-bromo-3-methylphenol in step-1 and (2-fluoropyridin-5-yl)boronic acid in step-2.

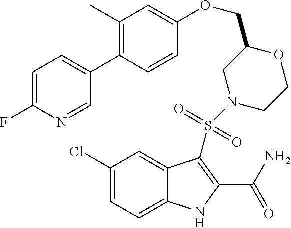 [SMILES: Cc1cc(OC[C@@H]2CN(S(=O)(=O)c3c(C(N)=O)[nH]c4ccc(Cl)cc34)CCO2)ccc1-c1ccc(F)nc1]

¹H NMR (DMSO-d₆, 300 MHz): δ 13.02 (s, 1H), 8.23 (d, J=12.6 Hz, 2H), 8.14 (d, J=1.8 Hz, 1H), 7.93 (m, 1H), 7.88 (d, J=1.8 Hz, 1H), 7.57 (d, J=8.7 Hz, 1H), 7.36 (dd, J=9.0 and 2.1 Hz, 1H), 7.22 (dd, J=8.4 and 2.7 Hz, 1H), 7.13 (d, J=8.4 Hz, 1H), 6.86 (d, J=2.1 Hz, 1H), 6.80 (dd, J=8.4 and 2.4 Hz, 1H), 3.99 (d, J=4.8 Hz, 2H), 3.92 (dd, J=11.1 Hz, 1H), 3.80 (m, 1H), 3.71 (d, J=11.4 Hz, 1H), 3.54 (m, 2H), 2.40 (t, J=11.1 Hz, 2H), 2.16 (s, 3H).

MS: (m/z) 559 [M+H]

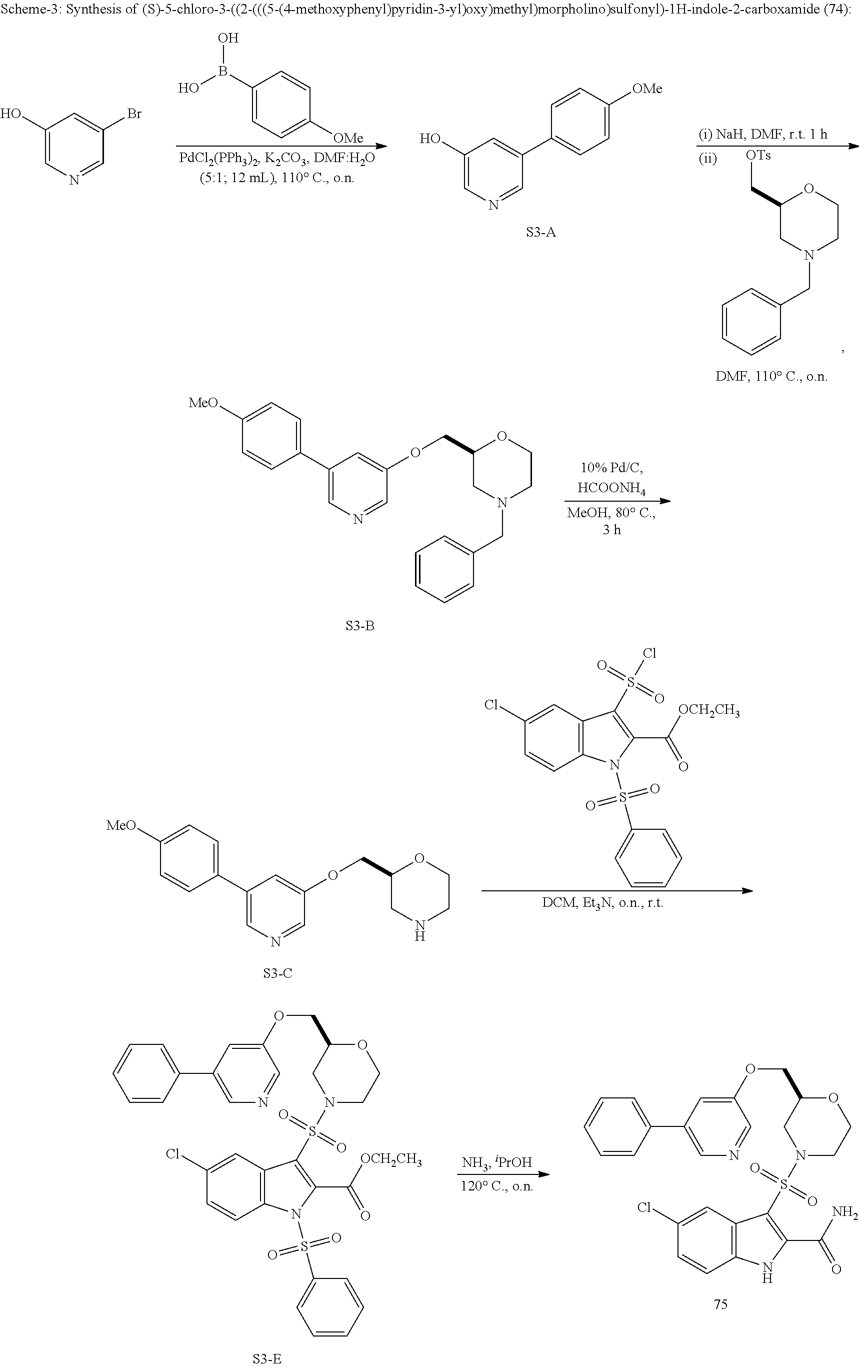

### 1. Preparation of 5-(4-methoxyphenyl)pyridin-3-ol (S3-A)

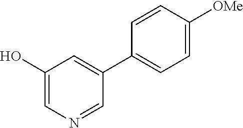 [SMILES: COc1ccc(-c2cncc(O)c2)cc1]

A mixture of 5-bromopyridine-3-ol (1 eq), 4-methoxyphenylboronic acid (1.2 eq) and potassium carbonate (1.4 eq) in DMF:H₂O (2:1) (12 mL) were sonicated for 2-3 minutes and stirred under argon atmosphere at an ambient temperature. The dichlorobis(triphenylphosphine)palladium(II) (0.1 eq) was added to the reaction mixture and stirred at 120° C. until complete consumption of the starting materials was indicated by TLC analysis (˜16 h). The reaction was diluted with EtOAc (50 mL) and filtered through a celite bed. The filtrate was washed with water (20 mL) and brine (5 mL). The organic layer was dried over anhydrous Na₂SO₄, filtered, concentrated in vacuo and the residue obtained was purified by flash column chromatography (200-400 mesh size silica gel, 30% ethyl acetate/pet. ether, gradient elution) to furnish 5-(4-methoxyphenyl)pyridin-3-ol as a gummy oil. ¹H NMR (CDCl₃, 300 MHz): δ 9.60 (s, 1H), 8.27 (s, 1H), 8.17 (s, 1H), 7.47 (d, J=8.7 Hz, 2H), 7.31 (d, J=9 Hz, 1H), 6.96 (d, J=8.7 Hz, 2H), 3.81 (s, 3H)

MS: (m/z) 202 [M⁺+1]

### 2. Preparation of (S)-4-benzyl-2-((5-(4-methoxyphenyl)pyridin-3-yl)oxy)methyl)morpholine (S3-B)

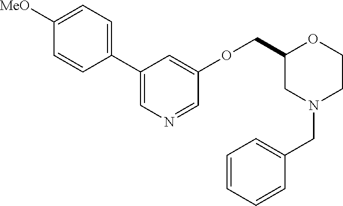

A solution of 5-(4-methoxyphenyl)pyridin-3-ol (1 eq) in DMF was added to a mixture of NaH (1 eq) in DMF at room temperature. The reaction mixture was initially stirred at ambient temperature for 10 minutes. The sodium alkoxide solution was then charged with (S)-(4-benzylmorpholin-2-yl)methyl 4-methylbenzenesulfonate (1 eq) in DMF and the resulting reaction mixture was heated at 110° C. for 3 h. The solution was concentrated under reduced pressure; residue obtained was diluted with ethyl acetate (50 mL) and washed with water (10 mL) and brine (5 mL). The combined organic layers were dried over anhydrous Na₂SO₄, filtered, concentrated in vacuo, and the residue obtained was purified by flash column chromatography (230-400 mesh size silica gel, 40% EtOAc/pet. ether gradient elution) to yield (S)-4-benzyl-2-((5-(4-methoxyphenyl)pyridin-3-yl)oxy)methyl)morpholine as a gummy oil. ¹H NMR (CDCl₃, 300 MHz): δ 8.44 (s, 1H), 8.26 (s, 1H), 7.53 (d, J=8.7 Hz, 2H), 7.36-7.29 (m, 6H), 7.02 (d, J=8.7 Hz, 2H), 4.12-3.94 (m, 4H), 3.87 (s, 3H), 3.84-3.77 (m, 1H), 3.59 (s, 2H), 2.93 (d, J=9.3 Hz, 1H), 2.77 (d, J=11.4 Hz, 1H), 2.34 (m, 1H), 2.17 (m, 1H).

MS: (m/z) 202 [M⁺+1]

### 3. Preparation of (S)-2-(((5-(4-methoxyphenyl)pyridin-3-yl)oxy)methyl)morpholine (S3-C)

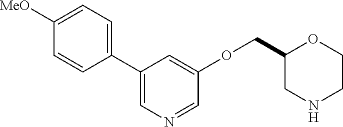

To a methanolic solution (20 mL) of (S)-4-benzyl-2-(((5-(4-methoxyphenyl)pyridin-3-yl)oxy)methyl)morpholine (1 eq) and ammonium formate (5 eq) in a two-necked round bottom flask was added Pd/C under argon atmosphere. The reaction mixture was refluxed till complete consumption of starting material as indicated by TLC (3 h). The reaction was filtered through celite bed and filtrate obtained was concentrated in vacuo and the residue obtained was purified by flash column chromatography (200-400 mesh size silica gel, 10% methanol/dichloromethane, gradient elution) to furnish (S)-2-(((5-(4-methoxyphenyl)pyridin-3-yl)oxy)methyl)morpholine as a gummy oil.

¹H NMR (CDCl₃, 300 MHz): δ 8.45 (s, 1H), 8.27 (s, 1H), 7.54 (d, J=8.7 Hz, 2H), 7.83 (s, 1H), 7.03 (d, J=8.7 Hz, 2H), 4.15-4.10 (m, 1H), 4.06-3.99 (m, 3H), 3.88 (s, 3H), 3.76-3.68 (m, 1H), 3.09 (d, J=10.8 Hz, 1H), 2.96-2.90 (m, 1H), 2.85-2.78 (m, 2H).

MS: (m/z) 301 [M⁺+1]

### 4. Preparation of (S)-ethyl 5-chloro-3-((2-((5-(4-methoxyphenyl)pyridin-3-yl)oxy)methyl)morpholino)sulfonyl)-1-(phenylsulfonyl)-1H-indole-2-carboxylate (S3-D)

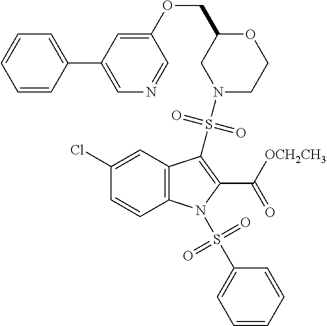

To a solution of (S)-2-(((5-(4-methoxyphenyl)pyridin-3-yl)oxy)methyl)morpholine (1 eq) in dichloromethane (10 mL), triethyl amine (5 eq) was added and the reaction mixture was stirred under argon atmosphere. Ethyl 5-chloro-3-(chlorosulfonyl)-1-(phenylsulfonyl)-1H-indole-2-carboxylate (1.2 eq) was added to reaction mixture and the resulting reaction mixture was stirred for approximately 16 h at ambient temperature. The reaction was concentrated in vacuo, crude material was adsorbed on silica gel and subjected to column chromatography (200-400 mesh size) using 5% methanol/dichloromathane as gradient eluent to furnish (S)-ethyl 5-chloro-3-((2-((5-(4-methoxyphenyl)pyridin-3-yl)oxy)methyl)morpholino)sulfonyl)-1-(phenylsulfonyl)-1H-indole-2-carboxylate as a yellowish solid.

¹H NMR (CDCl₃, 300 MHz): δ 8.47 (d, J=1.5 Hz, 1H), 8.25 (d, J=2.7 Hz, 1H), 8.11 (d, J=7.8 Hz, 2H), 8.00 (d, J=12 Hz, 1H), 7.89 (s, 1H), 7.68 (t, J=7.2 Hz, 1H), 7.56 (m, 5H), 7.44 (dd, J=9 Hz, 1H), 7.37 (s, 1H), 7.03 (d, J=8.7 Hz, 2H), 4.61 (dd, J=7.2 and 6.9 Hz, 2H), 4.17-3.88 (m, 5H), 3.76 (s, 3H), 3.72 (m, 2H), 2.81-2.71 (m, 2H), 1.49 (t, J=7.2 Hz, 3H).

MS: (m/z) 725 [M⁺+1]

### 5. Preparation of (S)-5-chloro-3-((2-((5-(4-methoxyphenyl)pyridin-3-yl)oxy)methyl)morpholino)sulfonyl)-1H-indole-2-carboxamide (74)

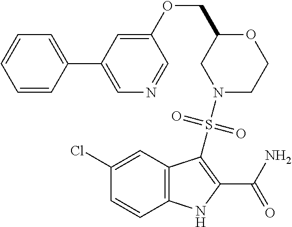

To a solution of (S)-ethyl 5-chloro-3-(2-(((5-(4-methoxyphenyl)pyridin-3-yl)oxy)methyl)morpholino)sulfonyl)-1-(phenylsulfonyl)-1H-indole-2-carboxylate (1 eq) in isopropyl alcohol (20 mL) at 0° C. ammonia gas was bubbled for 15 minutes. The resulting solution was sealed in seal-tube placed in an oil bath at 120° C. for 16 h. The solvent was removed under reduced pressure. Crude residue was subjected to flash column chromatography (200-400 mesh size silica gel, 10% methanol/dichloromethane, gradient elution) to yield (S)-5-chloro-3-((2-((5-(4-methoxyphenyl)pyridin-3-yl)oxy)methyl)morpholino)sulfonyl)-1H-indole-2-carboxamide (74) as a yellowish solid.

¹H NMR (CDCl₃, 300 MHz): δ 13.02 (s, 1H), 8.43 (s, 1H), 8.25 (d, J=12.6 Hz, 2H), 8.18 (s, 1H), 7.91 (s, 1H), 7.69 (d, J=8.4 Hz, 2H), 7.59 (m, 2H), 7.30 (d, J=9 Hz, 1H), 7.05 (d, J=8.4 Hz, 2H), 4.16 (s, 2H), 3.95-3.71 (m, 3H), 3.80 (s, 3H), 3.61 (m, 2H), 2.41 (m, 2H).

MS: (m/z) 557 [M+H]

### Preparation of (S)-5-chloro-3-((2-((5-(2-fluoro-5-(trifluoromethyl)phenyl)pyridin-3-yl)oxy)methyl)morpholino)sulfonyl)-1H-indole-2-carboxamide (75)

Synthesized by similar route as represented in scheme-3 by using (2-fluoro-5-(trifluoromethyl)phenyl)boronic acid in step-1.

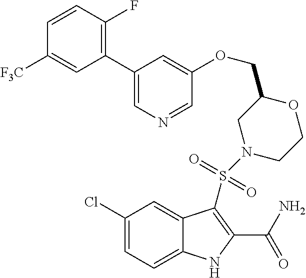 [SMILES: NC(=O)c1[nH]c2ccc(Cl)cc2c1S(=O)(=O)N1CCO[C@H](COc2cncc(-c3cc(C(F)(F)F)ccc3F)c2)C1]

¹H NMR (CDCl₃, 300 MHz): δ 11.81 (s, 1H), 8.87 (s, 1H), 8.39 (s, 1H), 8.31 (s, 1H), 8.10 (s, 1H), 7.69 (m, 3H), 7.54 (d, J=8.7 Hz, 1H), 7.33 (d, J=15 Hz, 2H), 6.30 (s, 1H), 4.08 (m, 4H), 3.77-3.66 (m, 2H), 3.60 (d, J=11.4 Hz, 1H), 2.56-2.41 (m, 2H).

MS: (m/z) 613 [M+H]

### Preparation of (S)-5-chloro-3-((2-(((5-phenylpyridin-3-yl)oxy)methyl)morpholino)sulfonyl)-1H-indole-2-carboxamide (76)

Synthesized by similar route as represented in scheme-3 by using phenylboronic acid in step-1.

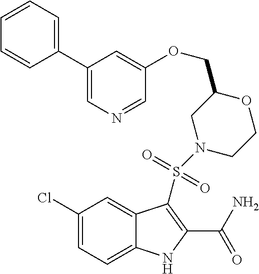 [SMILES: NC(=O)c1[nH]c2ccc(Cl)cc2c1S(=O)(=O)N1CCO[C@H](COc2cncc(-c3ccccc3)c2)C1]

¹H NMR (DMSO-d₆, 300 MHz): δ 13.04 (s, 1H), 8.49-8.48 (d, J=1.5 Hz, 1H), 8.25-8.20 (m, 3H), 7.90-7.89 (d, J=1.8 Hz, 1H), 7.74-7.72 (d, J=6.9 Hz, 2H), 7.64 (s, 1H), 7.59-7.56 (d, J=8.7 Hz, 1H), 7.51-7.42 (m, 3H), 7.37-7.34 (dd, J=1.8 Hz, 1H), 4.18 (d, J=4.8 Hz, 2H), 3.95-3.91 (m, 4H), 3.74-3.70 (d, J=11.1 Hz, 2H), 3.62-3.55 (m, 1H).

MS: (m/z) 527 [M+H]

### Preparation of (S)-5-chloro-3-((2-(((5-phenylpyrazin-2-yl)oxy)methyl)morpholino)sulfonyl)-1H-indole-2-carboxamide (77)

Synthesized by similar route as represented in scheme-3 by using 5-bromopyrazin-2-ol and phenylboronic acid in step-1.

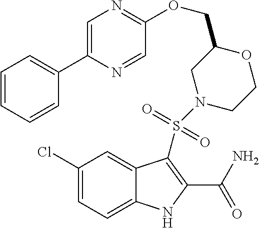 [SMILES: NC(=O)c1[nH]c2ccc(Cl)cc2c1S(=O)(=O)N1CCO[C@H](COc2cnc(-c3ccccc3)cn2)C1]

¹H NMR (DMSO-d₆, 300 MHz): δ 13.02 (br. s, 1H), 8.76 (d, J=1.2 Hz, 1H), 8.36 (d, J=1.2 Hz, 1H), 8.25 (d, J=14.7 Hz, 2H), 8.03 (d, J=7.2 Hz, 2H), 7.89 (d, J=1.8 Hz, 1H), 7.58-7.42 (m, 3H), 7.36 (dd, J=8.7 and 1.8 Hz, 1H), 7.51 (d, J=9.0 Hz, 1H), 5.11 (m, 1H), 4.13 (m, 2H), 3.91 (m, 3H), 3.56-3.50 (m, 3H).

MS: (m/z) 528 [M+H]

### Preparation of (S)-5-chloro-3-((2-((5-(2-fluorophenyl)pyridin-3-yl)oxy)methyl)morpholino)sulfonyl)-1H-indole-2-carboxamide (78)

Synthesized by similar route as represented in scheme-3 by using 2-fluorophenylboronic acid in step-1.

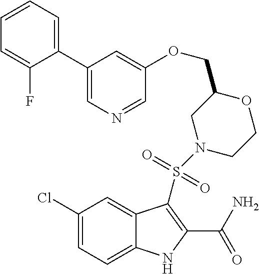 [SMILES: NC(=O)c1[nH]c2ccc(Cl)cc2c1S(=O)(=O)N1CCO[C@H](COc2cncc(-c3ccccc3F)c2)C1]

¹H NMR (DMSO-d₆, 300 MHz): δ 12.92 (s, 1H), 8.32-8.18 (m, 4H), 7.88 (s, 1H), 7.60-7.53 (m, 2H), 7.49-7.42 (m, 2H), 7.36-7.28 (m, 3H), 4.13 (d, J=4.8 Hz, 2H), 3.92 (m, 2H), 3.71 (d, J=11.1 Hz, 1H), 3.54-3.48 (m, 2H), 2.47-2.34 (m, 2H).

MS: (m/z) 545 [M+H]

### Preparation of (S)-5-chloro-3-((2-(((6-phenylpyridin-3-yl)oxy)methyl)morpholino)sulfonyl)-1H-indole-2-carboxamide (79)

Synthesized by similar route as represented in scheme-3 by using 6-bromopyridin-3-ol and phenylboronic acid in step-1.

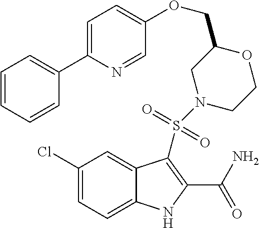 [SMILES: NC(=O)c1[nH]c2ccc(Cl)cc2c1S(=O)(=O)N1CCO[C@H](COc2ccc(-c3ccccc3)nc2)C1]

¹H NMR (CDCl₃, 300 MHz): δ 12.03 (br. s, 1H), 8.79 (br. s, 1H), 8.28 (d, J=2.7 Hz, 1H), 8.05 (d, J=1.5 Hz, 1H), 7.85 (s, 1H), 7.83 (s, 1H), 7.60 (d, J=8.7 Hz, 1H), 7.51 (d, J=9.0 Hz, 1H), 7.40-7.35 (m, 2H), 7.32-7.27 (m, 2H), 7.20 (dd, J=8.7 and 3.0 Hz, 1H), 6.32 (br. s, 1H), 4.02-3.91 (m, 4H), 3.72-3.65 (m, 2H), 3.62 (d, J=11.4 Hz, 1H), 2.53-2.35 (m, 2H).

MS: (m/z) 527 [M+H]

### Preparation of (S)-5-chloro-3-((2-(((6-phenylpyridin-2-yl)oxy)methyl)morpholino)sulfonyl)-1H-indole-2-carboxamide (80)

Synthesized by similar route as represented in scheme-3 by using 6-bromopyridin-2-ol in and phenylboronic acid in step-1.

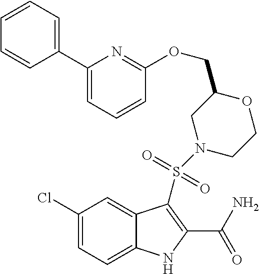 [SMILES: NC(=O)c1[nH]c2ccc(Cl)cc2c1S(=O)(=O)N1CCO[C@H](COc2cccc(-c3ccccc3)n2)C1]

¹H NMR (CDCl₃, 300 MHz): δ 9.02 (s, 1H), 8.12 (s, 1H), 7.98-7.95 (d, J=6.9 Hz, 2H), 7.65 (d, J=7.8 Hz, 1H), 7.47-7.34 (m, 6H), 6.69 (d, J=8.4 Hz, 1H), 5.95 (s, 1H), 4.54-4.51 (m, 1H), 4.41-4.36 (m, 1H), 4.02 (d, J=9.9 Hz, 2H), 3.79 (t, J=11.7 Hz, 2H), 3.62 (d, J=11.4 Hz, 1H), 2.61 (t, J=11.4 Hz, 1H), 2.49 (t, J=10.8 Hz, 1H).

MS: (m/z) 527 [M+H]

### Preparation of (S)-5-chloro-3-((2-(((5-phenylpyridin-2-yl)oxy)methyl)morpholino)sulfonyl)-1H-indole-2-carboxamide (81)

Synthesized by similar route as represented in scheme-3 by using 5-bromopyridin-2-ol and phenylboronic acid in step-1.

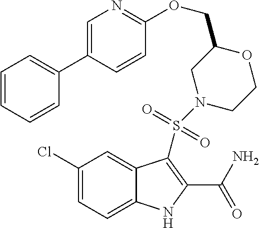 [SMILES: NC(=O)c1[nH]c2ccc(Cl)cc2c1S(=O)(=O)N1CCO[C@H](COc2ccc(-c3ccccc3)cn2)C1]

¹H NMR (CDCl₃, 300 MHz): δ 10.52 (br. s, 1H), 8.95 (br. s, 1H), 8.12 (d, J=1.8 Hz, 1H), 7.62 (dd, J=9.6, 1.5 Hz, 1H), 7.49 (d, J=8.7 Hz, 1H), 7.39 (s, J=2.1 Hz, 1H), 7.39-7.28 (m, 3H), 6.67 (d, J=9.6, 1H), 6.21 (br. s, 1H), 4.35 (dd, J=13.8 and 3.0 Hz, 1H), 3.39-3.53 (m, 6H), 2.49 (dt, J=11.4 and 3.0 Hz, 1H), 2.28 (t, J=9.9 Hz, 1H),

MS: (m/z) 527 [M+H]

### Preparation of (S)-5-chloro-3-((2-((4-(2,6-dimethylpyridin-4-yl)phenoxy)methyl)morpholino)sulfonyl)-1H-indole-2-carboxamide (82)

Synthesized by similar route as represented in scheme-3 by using 4-hydroxyphenylboronic acid and 4-bromo-2,6-dimethylpyridine in step-1.

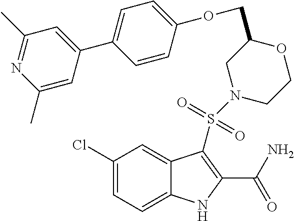 [SMILES: Cc1cc(-c2ccc(OC[C@@H]3CN(S(=O)(=O)c4c(C(N)=O)[nH]c5ccc(Cl)cc45)CCO3)cc2)cc(C)n1]

¹H NMR (CDCl₃, 300 MHz): δ 13.03 (br. s, 1H), 8.26 (br. s, 1H), 8.22 (br. s, 1H), 7.91 (d, J=2.1 Hz, 1H), 7.70 (d, J=8.7 Hz, 2H), 7.59 (d, J=8.7 Hz, 1H), 7.39 (dd, J=8.7 and 2.1 Hz, 1H), 7.31 (s, 2H), 7.02 (d, J=8.7 Hz, 2H), 4.04 (d, J=4.8 Hz, 2H), 3.95-3.52 (m, 5H), 2.45 (s, 6H), 2.59-2.44 (m, 2H).

MS: (m/z) 555 [M+H]

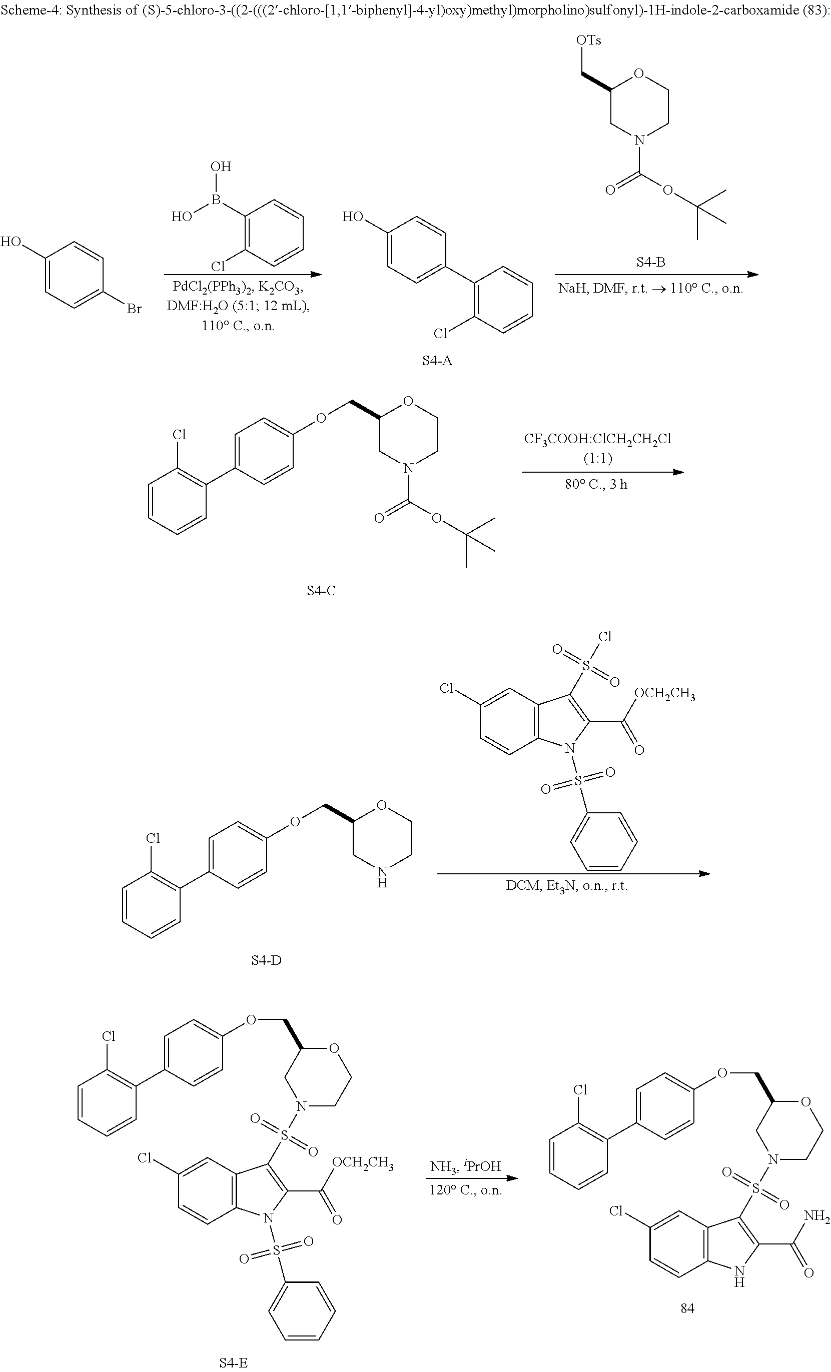

### 1. Preparation of 2′-chloro-[1,1′-biphenyl]-4-ol (S4-A)

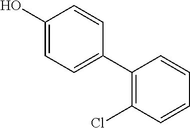 [SMILES: Oc1ccc(-c2ccccc2Cl)cc1]

A mixture of 4-bromophenol (1 eq), 2-chlorophenylboronic acid (1.2 eq) and potassium carbonate (1.4 eq) in DMF:H₂O (2:1) (12 mL) were sonicated for 2-3 minutes and stirred under argon atmosphere at an ambient temperature. The dichlorobis(triphenylphosphine)palladium(II) (0.1 eq) was added to the reaction mixture and stirred at 120° C. until complete consumption of the starting materials was indicated by TLC analysis (approximately 3 h). The reaction was diluted with EtOAc (50 mL) and filtered through a celite bed. The filtrate was washed with water (20 mL) and brine (5 mL). The organic layer was dried over anhydrous Na₂SO₄, filtered, concentrated in vacuo and the residue obtained was purified by flash column chromatography (200-400 mesh size silica gel, 30% ethyl acetate/pet. ether, gradient elution) to provide 2′-chloro-[1,1′-biphenyl]-4-ol as a gummy oil.

¹H NMR (300 MHz, CDCl₃): δ 7.46 (d, J=7.2 Hz, 1H), 7.34-7.21 (m, 5H), 6.90 (d, J=8.4 Hz, 2H).

### 2. Preparation of (S)-tert-butyl 2-(((2′-chloro-[1,1′-biphenyl]-4-yl)oxy)methyl)morpholine-4-carboxylate (S4-C)

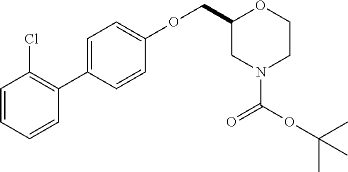 [SMILES: CC(C)(C)OC(=O)N1CCO[C@H](COc2ccc(-c3ccccc3Cl)cc2)C1]

A solution of 2′-chloro-[1,1′-biphenyl]-4-ol (1 eq) in DMF was added to a mixture of NaH (1 eq) in DMF at room temperature. The reaction mixture was initially stirred at ambient temperature for 1 h and was stirred again at 80° C. for 2 h. The sodium alkoxide solution was then charged with (S)-tert-butyl 2-((tosyloxy)methyl)morpholine-4-carboxylate (1 eq) in DMF and the resulting reaction mixture was heated at 110° C. for 16 h. The solution was concentrated under reduced pressure; residue obtained was diluted with ethyl acetate (50 mL) and washed with water (10 mL) and brine (5 mL). The combined organic layers were dried over anhydrous Na₂SO₄, filtered, concentrated in vacuo, and the residue obtained was purified by flash column chromatography (230-400 mesh size silica gel, 40% EtOAc/pet. ether gradient elution) to yield (S)-tert-butyl 2-(((2′-chloro-[1,1′-biphenyl]-4-yl)oxy)methyl)morpholine-4-carboxylate as a gummy oil.

¹H NMR (CDCl₃, 300 MHz): δ 7.45 (d, J=7.2 Hz, 1H), 7.37 (d, J=8.7 Hz, 2H), 7.32-7.20 (m, 3H), 6.98 (d, J=8.7 Hz, 2H), 4.07-4.02 (m, 1H), 3.96-3.86 (m, 3H), 3.73 (t, J=11.1 Hz, 1H), 3.08 (d, J=11.4 Hz, 1H), 2.98 (t, J=12.3 Hz, 1H), 2.86-2.73 (m, 2H), 1.47 (s, 9H).

MS: (m/z) 404 [M⁺+1]

### 3. Preparation of (S)-2-(((2′-chloro-[1,1′-biphenyl]-4-yl)oxy)methyl)morpholine (S4-D)

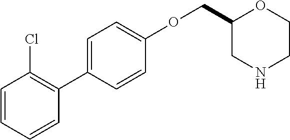 [SMILES: Clc1ccccc1-c1ccc(OC[C@@H]2CNCCO2)cc1]

(S)-tert-butyl 2-(((2′-chloro-[1,1′-biphenyl]-4-yl)oxy)methyl)morpholine-4-carboxylate was dissolved in CF₃COOH:1,2-dichloroethane (1:1, 6 mL) and the solution was heated at 80° C. for 2 h under argon atmosphere. Upon completion of reaction as indicated by TLC, the reaction mixture was concentrated in vacuo. The residue was adsorbed on silica gel and subjected to a column chromatography (200-400 mesh size silica gel) using 10% methanol/dichloromethane as gradient eluent to furnish (S)-2-(((2′-chloro-[1,1′-biphenyl]-4-yl)oxy)methyl)morpholine as a gummy oil.

¹H NMR (CDCl₃, 300 MHz): δ 7.45 (d, J=7.2 Hz, 1H), 7.37 (d, J=8.7 Hz, 2H), 7.32-7.20 (m, 3H), 6.98 (d, J=8.7 Hz, 2H), 4.07-4.02 (m, 1H), 3.96-3.86 (m, 3H), 3.73 (t, J=11.1 Hz, 1H), 3.08 (d, J=11.4 Hz, 1H), 2.98 (t, J=12.3 Hz, 1H), 2.86-2.73 (m, 2H).

MS: (m/z) 304 [M⁺+1]

### 4. Preparation of (S)-ethyl 5-chloro-3-((2-(((2′-chloro-[1,1′-biphenyl]-4-yl)oxy)methyl)morpholino)sulfonyl)-1-(phenylsulfonyl)-1H-indole-2-carboxylate (S4-E)

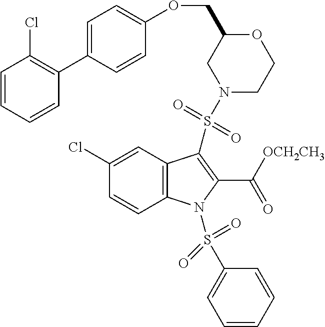

To a solution of (S)-2-(((5-(4-methoxyphenyl)pyridin-3-yl)oxy)methyl)morpholine (1 eq) in dichloromethane (10 mL), triethyl amine (5 eq) was added and the reaction mixture was stirred under argon atmosphere. Ethyl 5-chloro-3-(chlorosulfonyl)-1-(phenylsulfonyl)-1H-indole-2-carboxylate (1.2 eq) was added to the reaction mixture and the resulting reaction mixture was stirred for ˜16 h at ambient temperature. The reaction was concentrated in vacuo, crude material was adsorbed on silica gel and subjected to column chromatography (200-400 mesh size) using 5% methanol/dichloromathane as gradient eluent to furnish (S)-ethyl 5-chloro-3-((2-(((2′-chloro-[1,1′-biphenyl]-4-yl)oxy)methyl)morpholino)sulfonyl)-1-(phenylsulfonyl)-1H-indole-2-carboxylate as a yellowish solid.

¹H NMR (CDCl₃, 300 MHz): δ 8.09 (d, J=7.5 Hz, 2H), 7.98 (d, J=9 Hz, 1H), 7.87 (d, J=1.8 Hz, 1H), 7.66 (t, J=7.5 Hz, 1H), 7.53 (d, J=8.1 Hz, 2H), 7.48 (d, J=7.5 Hz, 1H), 7.43 (t, J=2.1 Hz, 1H), 7.40-7.35 (m, 3H), 7.32-7.21 (m, 2H), 6.95 (d, J=8.7 Hz, 2H), 4.59 (q, J=7.2 Hz, 2H), 4.09-3.89 (m, 5H), 3.73 (t, J=12 Hz, 2H), 2.82 (t, J=9.0 Hz, 1H), 2.69 (t, J=10.2 Hz, 1H), 1.47 (t, J=7.2 Hz, 3H).

MS: (m/z) 729 [M⁺+1]

### 5. Preparation of (S)-5-chloro-3-((2-(((2′-chloro-[1,1′-biphenyl]-4-yl)oxy)methyl)morpholino)sulfonyl)-1H-indole-2-carboxamide (83)

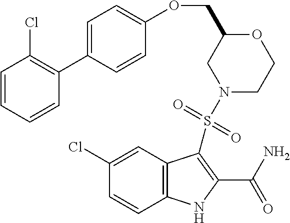

To a solution of (S)-ethyl 5-chloro-3-((2-(((2′-chloro-[1,1′-biphenyl]-4-yl)oxy)methyl)morpholino)sulfonyl)-1-(phenylsulfonyl)-1H-indole-2-carboxylate (1 eq) in isopropyl alcohol (20 mL) at 0° C. ammonia gas was bubbled for 15 minutes. The resulting solution was sealed in seal-tube and placed in an oil bath at 120° C. for 16 h. The solvent was removed under reduced pressure. Crude residue was subjected to flash column chromatography (200-400 mesh size silica gel, 10% methanol/dichloromethane, gradient elution) to yield (S)-5-chloro-3-((2-((2′-chloro-[1,1′-biphenyl]-4-yl)oxy)methyl)morpholino)sulfonyl)-1H-indole-2-carboxamide (83) as a yellowish solid.

¹H NMR (CDCl₃, 300 MHz): δ 10.50 (s, 1H), 9.06 (s, 1H), 8.14 (s, 1H), 7.94 (d, J=6.9 Hz, 2H), 7.61-7.43 (m, 3H), 7.38-7.33 (m, 2H), 7.29-7.25 (m, 2H), 6.91 (d, J=8.4 Hz, 2H), 4.05-3.90 (m, 4H), 3.81-3.71 (m, 2H), 3.62 (d, J=11.7 Hz, 1H), 2.63-2.55 (m, 1H), 2.49 (dd, J=10.8 and 9.9 Hz, 1H).

MS: (m/z) 557 [M−2H]

### Preparation of (S)-5-chloro-3-((2-((4-(2-chloropyridin-4-yl)phenoxy)methyl)morpholino)sulfonyl)-1H-indole-2-carboxamide (84)

Synthesized by similar route as represented in scheme-4 by using 2-chloropyridin-4-ylboronic acid in step-1.

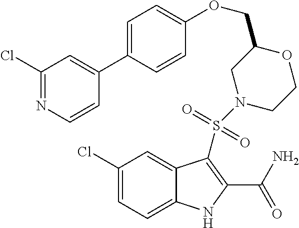 [SMILES: NC(=O)c1[nH]c2ccc(Cl)cc2c1S(=O)(=O)N1CCO[C@H](COc2ccc(-c3ccnc(Cl)c3)cc2)C1]

¹H NMR (CDCl₃, 300 MHz): δ 8.38 (d, J=5.4, 1H), 8.23 (d, J=13.5, 2H), 7.87 (s, 1H), 7.79 (t, J=4.5, 3H), 7.69 (d, J=5.4, 1H), 7.57 (d, J=9, 1H), 7.35 (d, J=8.7, 1H), 7.02 (d, J=8.7, 2H), 4.04 (d, J=4.8, 2H), 3.88-3.68 (m, 3H), 3.53 (d, J=12, 2H), 2.47-2.37 (m, 2H).

MS: (m/z) 561 [M+H]

### Preparation of (S)-5-chloro-3-((2-((3-(2-chloropyridin-4-yl)phenoxy)methyl)morpholino)sulfonyl)-1H-indole-2-carboxamide (85)

Synthesized by similar route as represented in scheme-4 by using 3-bromophenol and 2-chloropyridin-4-ylboronic acid in step-1

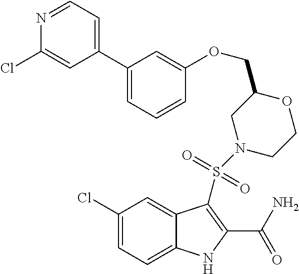 [SMILES: NC(=O)c1[nH]c2ccc(Cl)cc2c1S(=O)(=O)N1CCO[C@H](COc2cccc(-c3ccnc(Cl)c3)c2)C1]

¹H NMR (DMSO-d₆, 300 MHz): δ 13.02 (s, 1H), 8.44 (d, J=5.4 Hz, 1H), 8.24 (d, J=13.5 Hz, 2H), 7.89 (dd, J=12.9 and 1.8 Hz, 2H), 7.74 (dd, J=5.1 and 1.2 Hz, 1H), 7.57 (d, J=9.0 Hz, 1H), 7.38 (m, 4H), 7.03 (m, 1H), 4.08 (d, J=3.6 Hz, 2H), 3.94 (d, J=11.1 Hz, 1H), 3.85 (m, 1H), 3.73 (d, J=11.4 Hz, 1H), 3.54 (m, 2H), 2.40 (t, J=10.8 Hz, 2H).

MS: (m/z) 561 [M+H]

### Preparation of (S)-5-chloro-3-((2-((3-(6-chloropyridin-3-yl)phenoxy)methyl)morpholino)sulfonyl)-1H-indole-2-carboxamide (86)

Synthesized by similar route as represented in scheme-4 by using 3-bromophenol and 2-chloropyridin-5-ylboronic acid in step-1.

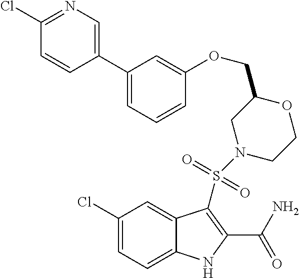 [SMILES: NC(=O)c1[nH]c2ccc(Cl)cc2c1S(=O)(=O)N1CCO[C@H](COc2cccc(-c3ccc(Cl)nc3)c2)C1]

¹H NMR (DMSO-d₆, 300 MHz): δ 13.01 (s, 1H), 8.71 (d, J=2.4 Hz, 1H), 8.24 (d, J=14.1 Hz, 2H), 8.16 (dd, J=8.4 and 2.4 Hz, 1H), 7.89 (dd, J=1.8 Hz, 1H), 7.59 (dd, J=8.4 and 4.2 Hz, 2H), 7.35 (m, 2H), 7.26 (m, 2H), 6.97 (dd, J=7.8 and 1.5 Hz, 1H), 4.06 (d, J=4.5 Hz, 2H), 3.94 (d, J=11.7 Hz, 1H), 3.85 (m, 1H), 3.72 (d, J=11.1 Hz, 1H), 3.54 (m, 2H), 2.40 (t, J=10.8 Hz, 2H).

MS: (m/z) 561 [M+H]

### Preparation of (S)-5-chloro-3-((2-((4-(2-chloropyridin-4-yl)-3-methylphenoxy)methyl)morpholino)sulfonyl)-1H-indole-2-carboxamide (87)

Synthesized by similar route as represented in scheme-4 by using 4-bromo-3-methylphenol and 2-chloropyridin-4-ylboronic acid in step-1.

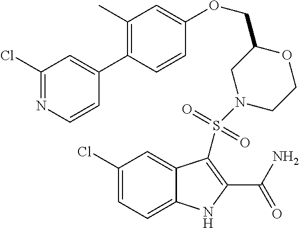 [SMILES: Cc1cc(OC[C@@H]2CN(S(=O)(=O)c3c(C(N)=O)[nH]c4ccc(Cl)cc34)CCO2)ccc1-c1ccnc(Cl)c1]

¹H NMR (DMSO-d₆, 300 MHz): δ 13.02 (s, 1H), 8.40 (d, J=5.1 Hz, 1H), 8.23 (d, J=13.2 Hz, 2H), 7.89 (d, J=1.8 Hz, 1H), 7.57 (d, J=8.7 Hz, 1H), 7.45 (s, 1H), 7.37 (m, 2H), 7.19 (d, J=8.4 Hz, 1H), 6.86 (d, J=1.8 Hz, 1H), 6.84 (dd, J=8.4 and 2.4 Hz, 1H), 4.08 (d, J=4.8 Hz, 2H), 3.93 (dd, J=11.7 Hz, 1H), 3.80 (m, 1H), 3.71 (d, J=11.4 Hz, 1H), 3.54 (m, 2H), 2.40 (t, J=11.8 Hz, 2H), 2.22 (s, 3H).

MS: (m/z) 575 [M+H]

### Preparation of (S)-5-chloro-3-((2-((4-(6-chloropyridin-3-yl)-3-methylphenoxy)methyl)morpholino)sulfonyl)-1H-indole-2-carboxamide (88)

Synthesized by similar route as represented in scheme-4 by using 4-bromo-3-methylphenol and 2-chloropyridin-5-ylboronic acid in step-1.

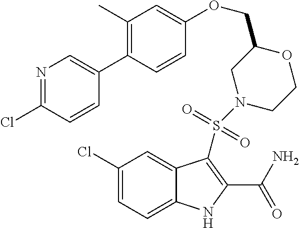 [SMILES: Cc1cc(OC[C@@H]2CN(S(=O)(=O)c3c(C(N)=O)[nH]c4ccc(Cl)cc34)CCO2)ccc1-c1ccc(Cl)nc1]

¹H NMR (DMSO-d₆, 300 MHz): δ 13.02 (s, 1H), 8.33 (d, J=2.1 Hz, 1H), 8.24 (d, J=13.5 Hz, 2H), 7.89 (d, J=1.5 Hz, 1H), 7.83 (dd, J=8.1 and 2.4 Hz, 1H), 7.57 (t, J=8.1 Hz, 2H), 7.37 (dd, J=9.0 and 1.8 Hz, 1H), 7.15 (d, J=8.4 Hz, 1H), 6.86 (br. s, 1H), 6.83 (m, 1H), 3.99 (d, J=4.8 Hz, 2H), 3.93 (dd, J=11.4 Hz, 1H), 3.80 (m, 1H), 3.71 (d, J=11.4 Hz, 1H), 3.54 (m, 2H), 2.40 (t, J=11.1 Hz, 2H), 2.17 (s, 3H).

MS: (m/z) 575 [M+H]

### Scheme-5: Synthesis of (S)-5-chloro-3-((2-((cinnolin-3-yloxy)methyl)morpholino)sulfonyl)-1H-indole-2-carboxamide (89)

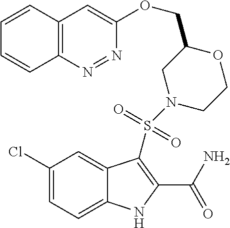 [SMILES: NC(=O)c1[nH]c2ccc(Cl)cc2c1S(=O)(=O)N1CCO[C@H](COc2cc3ccccc3nn2)C1]

Synthesized by a similar route as described earlier starting from a reaction between (S)-(4-benzylmorpholin-2-yl)methyl 4-methylbenzenesulfonate and cinnolin-3-ol [Scheme 3, step 2].

¹H NMR (DMSO-d₆, 300 MHz): δ 13.04 (s, 1H), 8.60 (d, J=2.7 Hz, 1H), 8.26 (d, J=14.1 Hz, 2H), 7.93 (m, 2H), 7.85 (m, 1H), 7.76 (d, J=2.7 Hz, 1H), 7.59 (s, 1H), 7.56 (m, 1H), 7.55 (s, 1H), 7.39 (dd, J=1.8 Hz, 1H), 4.2 (d, J=5.1 Hz, 2H), 3.93 (m, 2H), 3.75 (d, 1H), 3.56 (m, 2H) 1.58 (d, J=16.2 Hz, 2H).

MS: (m/z) 501 [M+H]

### Preparation of (S)-5-chloro-3-((2-((quinolin-8-yloxy)methyl)morpholino)sulfonyl)-1H-indole-2-carboxamide (90)

Synthesized by a similar route as described earlier starting from a reaction between (S)-(4-benzylmorpholin-2-yl)methyl 4-methylbenzenesulfonate and quinolin-8-ol [Scheme 3, step 2].

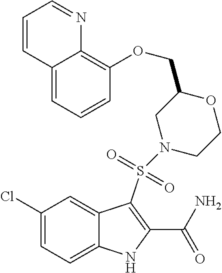 [SMILES: NC(=O)c1[nH]c2ccc(Cl)cc2c1S(=O)(=O)N1CCO[C@H](COc2cccc3cccnc23)C1]

¹H NMR (DMSO-d₆, 300 MHz): δ 13.03 (s, 1H), 8.83 (dd, J=4.2 and 1.5 Hz, 1H), 8.3 (dd, J=8.4 and 1.5 Hz, 2H), 8.21 (s, 1H), 7.90 (d, J=1.8 Hz, 1H), 7.59-7.43 (m, 4H), 7.37 (dd, J=8.7 and 1. Hz, 1H), 7.17 (d, J=6 Hz, 1H), 4.18 (br. s, 2H), 3.97 (d, J=9.9 Hz. 2H), 3.84 (d, J=11.1 Hz, 1H), 3.65 (m, 2H), 3.16 (s, 1H), 2.56 (s, 1H).

MS: (m/z) 501 [M+H]

### Preparation of (S)-5-chloro-3-((2-((quinazolin-4-yloxy)methyl)morpholino)sulfonyl)-1H-indole-2-carboxamide (91)

Synthesized by a similar route as described earlier starting from a reaction between (S)-(4-benzylmorpholin-2-yl)methyl 4-methylbenzenesulfonate and quinazolin-4-ol [Scheme 3, step 2].

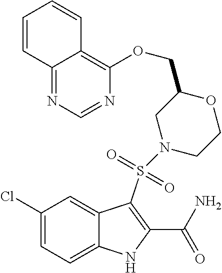 [SMILES: NC(=O)c1[nH]c2ccc(Cl)cc2c1S(=O)(=O)N1CCO[C@H](COc2ncnc3ccccc23)C1]

¹H NMR (DMSO-d₆, 300 MHz): δ 13.00 (s, 1H), 8.23 (s, 1H), 8.16 (s, 2H), 8.14 (s, 1H), 7.88 (d, J=1.8 Hz, 1H), 7.8 (m, 1H), 7.65 (d, J=8.1 Hz, 1H), 7.54 (m, 2H), 7.38 (dd, J=8.7 and 1.8 Hz, 1H), 4.2 (dd, J=13.8 and 3.3 Hz, 1H), 4.02 (t, J=7.8 Hz, 1H), 3.95 (d, J=11.7 Hz, 1H), 3.85 (br. s, 2H), 3.70 (d, J=11.4 Hz, 1H), 3.46 (t, J=11.7 Hz, 2H), 3.3 (s, 1H), 2.4 (m, 1H), 2.24 (m, 1H).

MS: (m/z) 502 [M+H] 391, 149

### Preparation of (S)-5-chloro-3-((2-(((1-methyl-3-phenyl-1H-pyrazol-5-yl)oxy)methyl)morpholino)sulfonyl)-1H-indole-2-carboxamide (92)

Synthesized by a similar route as described earlier starting from a reaction between (S)-(4-benzylmorpholin-2-yl)methyl 4-methylbenzenesulfonate and 1-methyl-3-phenyl-1H-pyrazol-5-ol [Scheme 3, step 2].

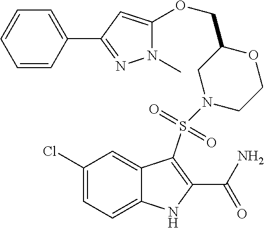 [SMILES: Cn1nc(-c2ccccc2)cc1OC[C@@H]1CN(S(=O)(=O)c2c(C(N)=O)[nH]c3ccc(Cl)cc23)CCO1]

¹H NMR (DMSO-d₆, 300 MHz): δ 13.02 (s, 1H), 8.22 (d, J=9.9 Hz, 2H), 7.9 (d, J=1.8 Hz, 1H), 7.69 (d, J=7.2 Hz, 2H), 7.5 (d, J=9.0 Hz, 1H), 7.34 (m, 3H), 7.24 (t, J=7.2 Hz, 1H), 6.1 (s, 1H), 4.10 (d, J=4.8 Hz, 2H), 3.92 (m, 2H), 3.72 (d, J=11.1 Hz, 1H), 3.59 (m, 2H), 3.45 (s, 3H), 2.39 (t, J=10.5 Hz, 2H).

MS: (m/z) 530 [M+H]

### Synthesis of Compound 51

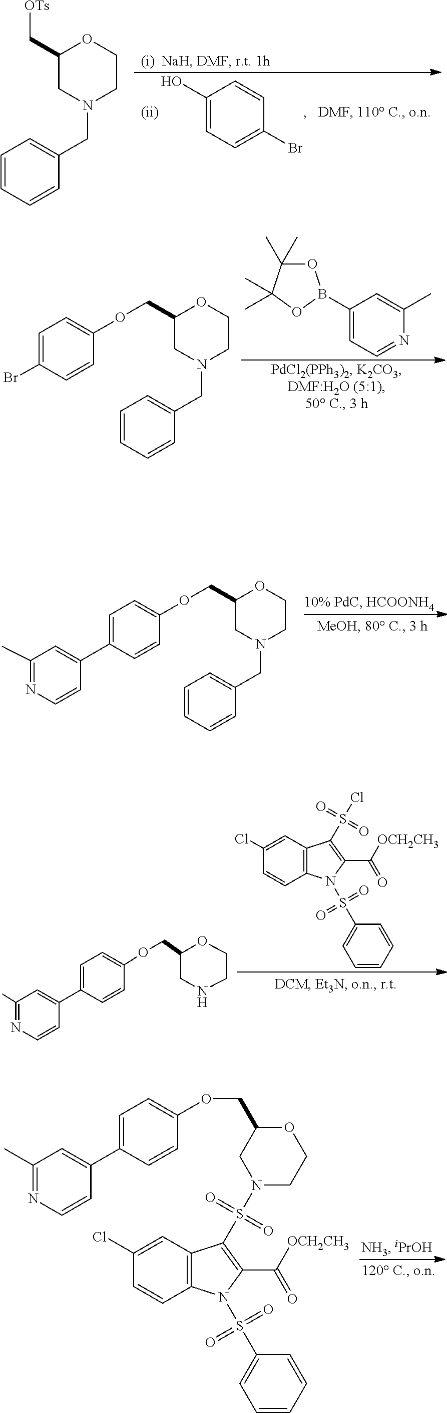

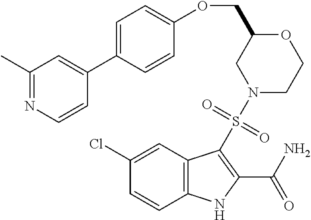 [SMILES: Cc1cc(-c2ccc(OC[C@@H]3CN(S(=O)(=O)c4c(C(N)=O)[nH]c5ccc(Cl)cc45)CCO3)cc2)ccn1]

### Step-1. Preparation of (S)-4-benzyl-2-((4-bromophenoxy)methyl)morpholine

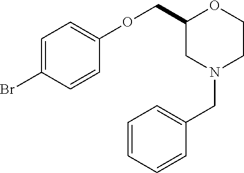

A solution of p-bromo phenol (1 eq) in DMF was added to a mixture of NaH (1 eq) in DMF at room temperature. The reaction mixture was initially stirred at ambient temperature for 1 h then heated to 80° C. for 2 h. The sodium alkoxide solution was then charged with (S)-(4-benzylmorpholin-2-yl)methyl 4-methylbenzenesulfonate (1 eq) in DMF and the resulting reaction mixture was heated at 110° C. for 16 h. The solution was concentrated under reduced pressure; residue obtained was diluted with ethyl acetate (50 mL) and washed with water (10 mL) and brine (5 mL). The combined organic layers were dried over anhydrous Na₂SO₄, filtered, concentrated in vacuo, and the residue obtained was purified by flash column chromatography (200-400 mesh size silica gel, 40% EtOAc/pet. ether gradient elution) to yield (S)-4-benzyl-2-((4-bromophenoxy)methyl)morpholine as a gummy oil. ¹H NMR (CDCl₃, 300 MHz): δ 7.44 (s, 1H), 7.39 (s, 1H), 7.33-7.22 (m, 5H), 6.92 (s, 1H), 6.86 (s, 1H), 3.9 (d, J=4.8 Hz, 1H), 3.82-3.81 (m, 2H), 3.57 (dd, J=11.1 and 2.4 Hz, 1H), 3.49 (s, 2H), 3.34 (s, 1H), 2.81 (d, J=11.4 Hz, 1H), 2.63 (d, J=11.1 Hz, 1H), 2.13 (dt, J=11.4 and 3.3 Hz, 1H), 2.00 (t, J=11.4 Hz, 1H).

### Step-2. Preparation of ((S)-4-benzyl-2-((4-(2-methylpyridin-4-yl)phenoxy)methyl)morpholine

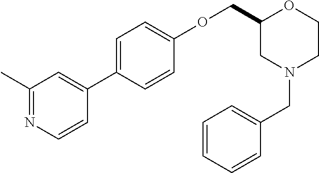

A mixture of (S)-4-benzyl-2-((4-bromophenoxy)methyl)morpholine (1 eq), 2-methyl-4-(4,4,5,5-tetramethyl-1,3,2-dioxaborolan-2-yl)pyridine (1.2 eq) and potassium carbonate (1.4 eq) in DMF:H₂O (5:1) were sonicated for 2-3 minutes and stirred under argon atmosphere at an ambient temperature. The dichlorobis(triphenylphosphine)Palladium(II) (0.1 eq) was added to the reaction mixture and stirred at 50° C. until complete consumption of the starting materials was indicated by TLC analysis (˜3 h). The reaction was diluted with EtOAc (50 mL) and filtered through a celite bed. The filtrate was washed with water (20 mL) and brine (5 mL). The organic layer was dried over anhydrous Na₂SO₄, filtered, concentrated in vacuo and the residue obtained was purified by flash column chromatography (200-400 mesh size silica gel, 30% ethyl acetate/pet. ether, gradient elution) to yield (S)-4-benzyl-2-((4-(2-methylpyridin-4-yl)phenoxy)methyl)morpholine as a gummy oil.

¹H NMR (DMSO-d₆, 300 MHz): δ 8.42 (d, J=5.1 Hz, 1H), 7.72 (d, J=8.7 Hz, 2H), 7.63-7.51 (m, 5H), 7.43 (d, J=5.1 Hz, 1H), 7.31-7.29 (m, 2H), 7.04 (d, J=9.0 Hz, 2H), 4.00-3.29 (m, 5H), 2.97-2.53 (m, 4H), 2.12-1.94 (m, 2H), 1.04 (s, 3H).

MS (m/z): 375 (M+H)

### Step-3. (S)-2-((4-(2-methylpyridin-4-yl)phenoxy)methyl)morpholine

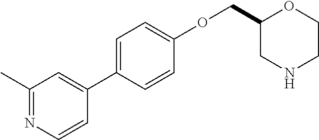

To a methanolic solution (20 mL) of (S)-4-benzyl-2-((4-(2-methylpyridin-4-yl)phenoxy)methyl)morpholine (1 eq) and ammonium formate (5 eq) in a two-necked round bottom flask, Pd/C was added under argon atmosphere. The reaction mixture was refluxed till complete consumption of starting material as indicated by TLC (3 h). The reaction was filtered through celite bed, filtrate obtained was concentrated in vacuo and the residue obtained was purified by flash column chromatography (200-400 mesh size silica gel, 10% methanol/dichloromethane, gradient elution) to yield (S)-2-((4-(2-methylpyridin-4-yl)phenoxy)methyl)morpholine as a gummy oil.

¹H NMR (CDCl₃, 300 MHz): δ 8.52 (d, J=5.1 Hz, 1H), 7.60 (d, J=8.7 Hz, 2H), 7.34 (d, J=15 Hz, 2H), 7.04 (d, J=8.7 Hz, 2H), 4.10 (m, 1H), 4.00-3.92 (m, 3H), 3.79-3.74 (dt, J=11.1 and 3 Hz, 1H), 3.11 (d, J=13 Hz, 1H), 2.99-2.80 (m, 3H), 2.62 (s, 3H).

MS (m/z): 285 (M+H)

### Step-4. (S)-ethyl 5-chloro-3-((2-((4-(2-methylpyridin-4-yl)phenoxy)methyl)morpholino)sulfonyl)-1-(phenylsulfonyl)-1H-indole-2-carboxylate

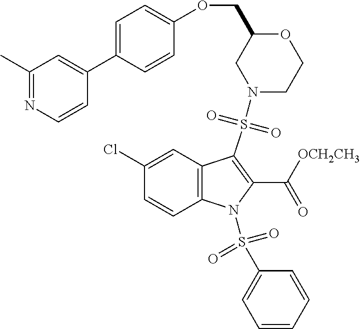 [SMILES: CCOC(=O)c1c(S(=O)(=O)N2CCO[C@H](COc3ccc(-c4ccnc(C)c4)cc3)C2)c2cc(Cl)ccc2n1S(=O)(=O)c1ccccc1]

To a solution of (S)-2-((4-(2-methylpyridin-4-yl)phenoxy)methyl)morpholine (1 eq) in dichloromethane, triethyl amine (5 eq) was added and the reaction mixture was stirred under argon atmosphere. Ethyl 5-chloro-3-(chlorosulfonyl)-1-(phenylsulfonyl)-1H-indole-2-carboxylate (1.2 eq) was added to reaction mixture and the resulting reaction mixture was stirred for ˜16 h at ambient temperature. The reaction was concentrated in vacuo, crude material was adsorbed on silica gel and subjected to column chromatography (200-400 mesh size) using 5% methanol/dichloromathane as gradient eluent to furnish (S)-ethyl 5-chloro-3-((2-((4-(2-methylpyridin-4-yl)phenoxy)methyl)morpholino)sulfonyl)-1-(phenylsulfonyl)-1H-indole-2-carboxylate as a yellowish solid.

¹H NMR (CDCl₃, 300 MHz): δ 8.52 (d, J=5.1 Hz, 1H), 8.12 (d, J=7.8 Hz, 1H), 8.00 (d, J=9.0 Hz, 1H), 7.89 (d, J=1.8 Hz, 1H), 7.70-7.52 (m, 5H), 7.45 (dd, J=9.0 and 1.8 Hz, 1H), 7.35 (s, 1H), 7.02 (d, J=7.8 Hz, 1H), 4.61 (q, J=7.2 Hz, 2H), 4.12-3.91 (m, 5H), 3.76-3.68 (m, 2H), 2.85-2.67 (m, 2H), 2.62 (s, 3H), 1.49 (t, J=7.2 Hz, 3H).

MS (m/z): 710 [M⁺]

### Step-5. (S)-5-chloro-3-((2-((4-(2-methylpyridin-4-yl)phenoxy)methyl)morpholino)sulfonyl)-1H-indole-2-carboxamide (52)

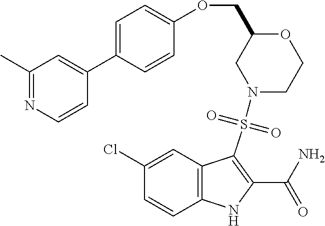 [SMILES: Cc1cc(-c2ccc(OC[C@@H]3CN(S(=O)(=O)c4c(C(N)=O)[nH]c5ccc(Cl)cc45)CCO3)cc2)ccn1]

To a solution of (S)-ethyl 5-chloro-3-((2-((4-(2-methylpyridin-4-yl)phenoxy)methyl)morpholino)sulfonyl)-1-(phenylsulfonyl)-1H-indole-2-carboxylate (1 eq) in isopropyl alcohol (20 mL) at 0° C. ammonia gas was bubbled for 15 minutes. The resulting solution was heated in a seal-tube and placed in an oil bath at 120° C. for 16 h. The solvent was removed under reduced pressure. Crude product was subjected to flash column chromatography (200-400 mesh size silica gel, 10% methanol/dichloromethane, gradient elution) to afford (S)-5-chloro-3-((2-((4-(2-methylpyridin-4-yl)phenoxy)methyl)morpholino)sulfonyl)-1H-indole-2-carboxamide (52) as a yellowish solid.

¹H NMR (CDCl₃, 300 MHz): δ 8.42-8.40 (d, J=5.4 Hz, 1H), 8.24-8.20 (d, J=13.5 Hz, 2H), 7.89-7.88 (d, J=1.5 Hz, 1H), 7.71-7.68 (d, J=8.7 Hz, 2H), 7.57-7.51 (m, J=8.7 Hz, 2H), 7.43-7.41 (d, J=5.1 Hz, 1H), 7.37-7.33 (dd, J=8.7 Hz, 1H), 7.01-6.98 (d, J=8.7 Hz, 2H), 4.03-4.01 (d, J=4.8 Hz, 2H), 3.89-3.68 (m, 3H), 3.54-3.50 (d, J=12.9 Hz, 2H), 2.48 (s, 3H), 2.41-2.34 (t, J=10.5 Hz, 2H).

MS (m/z): 541 [M+H]

### Example 2

### In vitro IGF-1R and IR Kinase Assays

The in vitro kinase assays using IGF-1R and IR kinase GST fusion proteins were conducted using a homogeneous time-resolved fluorescence (HTRF) format. Kinase reactions were carried out in a 384-well plate format in a final volume of 20 μL. The standard enzyme reaction buffer consisted of 50 mM Tris HCL (pH: 7.4), 1 mM EGTA, 10 mM MgCl₂, 2 mM DTT, 0.01% Tween-20, IGF-1R/IR kinase enzyme, poly GT peptide substrate (Perkin Elmer [Ulight Glu-Tyr (4:1)]n) and ATP [concentration equivalent to Kmapₚ] Inhibitors in DMSO (<1%), were added to give a final inhibitor concentration ranging from 40 μM to 40 pM. Briefly, 2.5 μL enzyme and 2.5 μL inhibitor was preincubated for 10 minutes at 23° C. followed by the addition of 2.5 μL of poly GT substrate (final concentration of 50 nM). Reaction was initiated with the addition of 2.5 μL of ATP (final concentration of 20 μM for IGF-1R assay and 10 μM for IR assay). After 1 hour incubation at 23° C., the kinase reaction was stopped with the addition of 5 μL EDTA (final concentration of 10 mM in 20 μL). Europium cryptate—labeled antiphosphotyrosine antibody PY20 (5 μL) was added (final concentration of 2 nM) and the mixture was allowed to equilibrate for 1 hour at 23° C. followed by reading the plate in an Envision plate reader.

The intensity of light emission at 665 nm was directly proportional to the level of substrate phosphorylation. The IC₅₀ values for inhibitors were determined by a four-parameter sigmoidal curve fit (Sigma plot or Graph pad).

IGFRK and IRK enzyme used for the assay was intracellular kinase domain of human IGF-1R and human IR cloned and expressed as GST fusion proteins using the baculovirus expression system and purified using glutathione—Sepharose column. IGFRK was used at a final concentration of 0.25 nM and IRK at 0.5 nM.

TABLE 1
In vitro IGF-1R kinase activity

| | IGF-1R |
|---|---|
| Compound | Activity |
| No. | IC₅₀(nM) |
| 1 | 33 |
| 2 | 8.5 |
| 3 | 31.2 |
| 4 | 11.8 |
| 5 | 30.4 |
| 6 | 65 |
| 7 | 15.1 |
| 8 | 15.5 |
| 9 | 220 |
| 10 | 199 |
| 11 | 15.4 |
| 12 | >100 |
| 13 | 3.4 |
| 14 | >100 |
| 15 | 3.7 |
| 16 | 2 |
| 17 | 10 |
| 18 | 7.3 |
| 19 | 2.9 |
| 20 | 140 |
| 21 | 184 |
| 22 | 42.5 |
| 23 | 132 |
| 24 | 293 |
| 25 | 53.3 |
| 26 | 11.2 |
| 27 | 4.7 |
| 28 | 6.9 |
| 29 | 43.2 |
| 30 | <100 |
| 31 | 8.7 |
| 32 | 3.5 |
| 33 | 30.2 |
| 34 | >1000 |
| 35 | >1000 |
| 36 | 81 |
| 37 | 166 |
| 38 | 113 |
| 39 | 206 |
| 40 | 361 |
| 41 | 184 |
| 42 | 119 |
| 43 | >100 |
| 44 | 46.4 |
| 45 | 12.1 |
| 46 | 12.8 |
| 47 | <100 |
| 48 | 4 |
| 49 | 5 |
| 50 | 3.2 |
| 51 | 4.3 |
| 52 | >100 |
| 53 | 6.4 |
| 54 | >1000 |
| 55 | >1000 |
| 56 | >1000 |
| 57 | >1000 |
| 58 | >1000 |
| 59 | >1000 |
| 60 | >1000 |
| 61 | >1000 |
| 62 | >1000 |
| 63 | >1000 |
| 64 | >1000 |
| 65 | >1000 |
| 66 | 18.7 |
| 67 | 4.3 |
| 68 | 37.9 |
| 69 | >100 |
| 70 | 75.9 |
| 71 | 5.3 |
| 72 | 13.3 |
| 73 | 30 |
| 74 | >1000 |
| 75 | >1000 |
| 76 | >1000 |
| 77 | 204 |
| 78 | >1000 |
| 79 | 25.4 |
| 80 | 47.1 |
| 81 | >1000 |
| 82 | 13.2 |
| 83 | 97 |
| 84 | 18.5 |
| 85 | 9.7 |
| 86 | 4.9 |
| 87 | 9.3 |
| 88 | 67 |
| 89 | 136 |
| 90 | >100 |
| 91 | >100 |
| 92 | >100 |

All the above compounds exhibit at least 30% inhibition at 1 uM.

### Example 3

### Anti-proliferative Assay

Anti-proliferative potential of compounds was tested using various cell lines (details provided in Table 2) by MTS (Promega, Cat # G1111), a tetrazolium compound ((3-(4,5-dimethylthiazol-2-yl)-5-(3-carboxymethoxyphenyl)-2-(4-sulphophenyl)-2Htetrazolium, inner salt; MTS) and Cell Counting kit-8 (CCK-8 a Dojindo's highly watersoluble tetrazolium salt of WST-8 [2-(2-methoxy-4-nitrophenyl)-3-(4-nitrophenyl)-5-(2,4-disulfophenyl)-2H-tetrazolium, monosodium salt]). MTS is a colorimetric assay for determining the number of viable cells in proliferation, cytotoxicity or chemosensitivity assays. This is used with an electron coupling reagent PMS (Phenazine methosulfate). MTS is bioreduced by cells into a formazan that is soluble in tissue culture medium. The absorbance of the formazan at 490 nm can be measured directly from 96 well assay plates without additional processing. Dehydrogenase enzymes found in metabolically active cells accomplish the conversion of MTS into the aqueous soluble formazan. The quantity of formazan product is directly proportional to the number of living cells in culture. In CCK-8, WST-8 is reduced by dehydrogenases in cells to give a yellow colored product formazan, which is measured at 450 nm.

For experimental purposes, cells were seeded at a density of 3000-5000 cells per well in 180 μL/well volume in transparent 96 well tissue culture plate (NUNC, USA) and incubated overnight at 37° C., 5% CO₂. Next day before adding compound the medium was replaced and 180 μL, of fresh medium added with the 100 ng/mL IGF without FCS followed by addition of 20 μL of 10× compound (10 mM stock made in DMSO and then further dilutions were made in medium, final DMSO concentration should not exceed 0.5%) and incubated for 72 hours in humidified 5% CO₂ incubator at 37±1° C. After incubation medium was replaced with 200 μL of medium containing 20 μL MTS reagent per well. Plates were incubated for 3-4 hours and absorbance was measured at 490 nm on Spectrophotometer (SpectraMax, Molecular Devices). Percentage cytotoxicity and IC₅₀ was calculated using SoftMax software. CCK-8 was used for suspension cell lines. Cell seeding and compound addition was done on same day. Following the incubation, 10 μL of CCK-8 solution was added in each well. After 4 hour incubation, the absorbance was determined at 450 nm using Spectrophotometer (SpectraMax, Molecular Devices). In every experiment, each condition was run in triplicate wells.

TABLE 2
Anti-proliferation IC₅₀ (nM)

| Cancer Cell | Compound | Compound | Compound | Compound |
|---|---|---|---|---|
| line | 51 | 1 | 7 | 999* |
| A673 | 0.15 | 1.8 | 0.6 | 3.5 |
| (Ewing's | | | | |
| Sarcoma) | | | | |

*Compound 999:

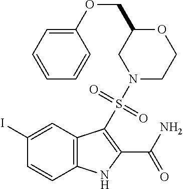 [SMILES: NC(=O)c1[nH]c2ccc(I)cc2c1S(=O)(=O)N1CCO[C@H](COc2ccccc2)C1]

As compared to compound 999, Compounds 51, 1 and 7 exhibit higher anti-proliferative activity in the Ewing's Sarcoma cell line.

### Example 4

### CYP Inhibition Fluorescence Assay

The % inhibition @ 10 uM data was generated from an rhCYP450/fluorescence assay according to the Vivid Invitrogen screening kits. The compounds were screened against 5 CYP450 (1A2, 2C9, 2C19, 2D6, and 3A4) isoforms. CYP3A4 constitutes 28% of the human hepatic CYP and is responsible for metabolism of 50% of all drugs. In this class of drugs, CYP3A4 is the most important CYP and thus it is highly desirable not to inhibit CYP3A4. The lower the percentage inhibition, the lower the CYP450 inhibitory liability of that specific compound.

TABLE 3
CYP data

| | % inhibition @ 10 uM | |
|---|---|---|
| Compound | 3A4 | 2D6 |
| 999 | 36 | 13 |
| 17 | 2 | 13 |
| 20 | 2 | 25 |
| 51 | 13 | 34 |
| 53 | 37 | 0 |
| 87 | 24 | 16 |

As compared to compound 999, compounds 17, 20, 51 and 87 exhibit reduced hepatic CYP3A4 inhibition. Compound 53 exhibits reduced hepatic CYP2D6 inhibition.

### Example 5

### Oral Absorption in Swiss Mice

Oral absorption of different compounds was ascertained in Swiss mice. The procedure followed was as detailed below:

Species: Swiss mice, overnight fasted state

Sex and weight: Male, in the weight range of 25-30 g

Number of animals: 18 animals divided into six groups (n=3) and bled as follows:

A: 4 h and 12 h

B: 2 h and 30 h

C: 1 h and 24 h

D: 30 min and 10 h

E: 15 min and 8 h

F: 5 min and 6 h

Dose: 10 mg/kg p.o.

Dosing volume: 10 mL/kg

Formulation: Compound suspended in 0.5% carboxymethylcellulose or 0.25% methylcellulose

Blood (250 μL) is collected from the retro-orbital sinus at designated time points from each mouse using heparinized bleeding capillaries into micro-centrifuge tubes containing 5 μL EDTA (200 mM) as anticoagulant. Plasma is separated by centrifugation at 10000 rpm for 5 min and samples stored at −70° C. until bioanalysis.

### Sample Preparation and Bioanalysis:

On the day of analysis, plasma samples are thawed at room temperature. An aliquot (100 μL) of each plasma sample is spiked individually with 10 μL of internal standard. The samples are then vortexed for 10 seconds followed by addition of 1 mL of extraction solvent and vortexed for 5 minutes. The samples are then centrifuged at 10000 rpm for 5 minutes at 4° C. Supernatants (800 μL) are removed and transferred to glass tubes and evaporated to dryness under nitrogen. The dried residues are reconstituted using 100 μL of 90:10% v/v acetonitrile:MilliQ water. The reconstituted samples are vortexed, centrifuged and injected into LC-MS/MS for analysis. The calibration curve is prepared in the range of 0.5 to 5000 ng/mL in mouse plasma for quantitation.

TABLE 4
Oral absorption of in Swiss mice at the dose of 10 mg/kg.

| | PK parameters | | |
|---|---|---|---|
| Compound | Cmax | AUC_{0-infinity} | |
| no | (μM) | (ng*h/mL) | T_{1/2} (h) |
| 999 | 1.2 | 1051 | 1.31 |
| 1 | 8.56 | 10856 | 5.3 |
| 3 | 2.98 | 1962 | 4.6 |
| 7 | 2.2 | 1937 | 0.9 |
| 20 | 1.98 | 4711 | 2.1 |
| 32 | 2.78 | 3193 | 2.8 |
| 84 | 1.24 | 2195 | 1.3 |
| 51 | 2.55 | 3252 | 1.0 |

As compared to compound 999, compounds 1, 3, 7, 20, 32, 84 and 51 exhibit higher AUC and Cmax values.

1. A compound represented by Formula I: 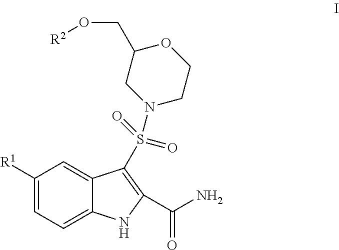
wherein:
R¹ is H, halo, or CN;
R² is 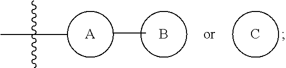
Ring A is phenyl or a 5 or 6-membered heteroaryl, which can be optionally substituted with one to three moieties selected from C₁-C₃ alkyl, C₁-C₃ haloalkyl, OR³, N(R^{a})₂ and halo;
Ring B is phenyl or a 5 or 6-membered heteroaryl, which can be optionally substituted with one to three moieties selected from C₁-C₃ alkyl, C₁-C₃ haloalkyl, OR³, N(Ra)₂ and halo;
Ring C is a 10-membered fused bicyclic aryl or heteroaryl, which can be optionally substituted with one to three moieties selected from C₁-C₃ alkyl and halo;
W is independently H or C₁-C₃ alkyl;
R³ is H, C₁-C₃ haloalkyl or C₁-C₃ alkyl;
Or a pharmaceutically acceptable salt thereof.

2. The compound of claim 1, wherein R² is

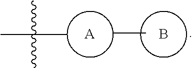

3. The compound of claim 2, wherein Ring A is phenyl, pyridyl, pyrazinyl, or pyrazolyl; Ring B is phenyl, pyridyl, or pyrimidinyl.

4. The compound of claim 2, wherein Ring A is phenyl; Ring B is phenyl or pyridyl.

5. The compound of claim 1, represented by Formula IA: 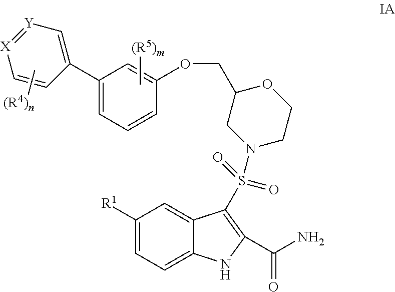
wherein
X is C or N;
Y is C or N; provided that X and Y are not both N;
R¹ is halo;
R⁴ is H, C₁-C₃ alkyl, C₁-C₃ haloalkyl, OR³, N(Ra)₂ or halo;
R⁵ is H, C₁-C₃ alkyl, C₁-C₃ haloalkyl, OR³, N(Ra)₂ or halo;
R^{a} is independently H or C₁-C₃ alkyl; and
R³ is H, C₁-C₃ haloalkyl or C₁-C₃ alkyl;
n is 0, 1, 2 or 3; and
mis 0, 1, 2 or 3.

6. The compound of claim 1, represented by Formula IB: 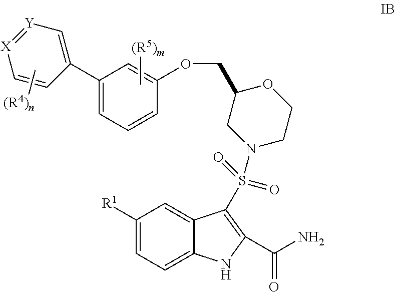
wherein
X is C or N;
Y is C or N; provided that X and Y are not both N;
R¹ is halo;
R⁴ is H, C₁-C₃ alkyl, C₁-C₃ haloalkyl, OR³, N(R^{a})₂ or halo;
R⁵ is H, C₁-C₃ alkyl, C₁-C₃ haloalkyl, OR³, N(R^{a})₂ or halo;
R^{a} is independently H or C₁-C₃ alkyl; and
R³ is H, C₁-C₃ haloalkyl or C₁-C₃ alkyl:
n is 0, 1, 2 or 3; and
m is 0, 1, 2 or 3.

7. The compound of claim 5, wherein
Y is N and X is C or X and Y are both C;
R¹ is Cl;
R⁴ is H, methyl, methoxy or halo;
R⁵ is H, methyl, methoxy or halo;
n is 0 or 1; and
m is 0 or 1.

8. The compound of claim 2, represented by Formula IIA: 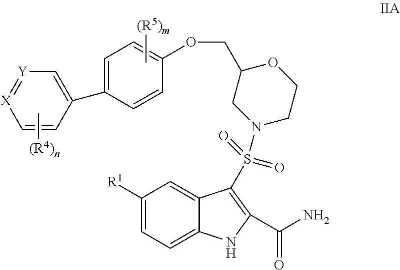
wherein
X is C or N;
Y is C or N; provided that X and Y are not both N;
R¹ is halo;
R⁴ is H, C₁-C₃ alkyl, C₁-C₃ haloalkyl, OR³, N(R^{a})₂ or halo;
R⁵ is H, C₁-C₃ alkyl, C₁-C₃ haloalkyl, OR³, N(R^{a})₂ or halo;
R^{a} is independently H or C₁-C₃ alkyl; and
R³ is H, C₁-C₃ haloalkyl or C₁-C₃ alkyl;
n is 0, 1, 2 or 3; and
m is 0, 1, 2 or 3.

9. The compound of claim 8, underrepresented by Formula IIB: 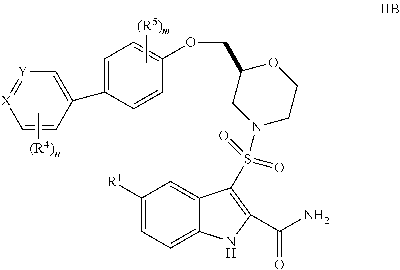
wherein
X is C or N;
Y is C or N; provided that X and Y are not both N;
R¹ is halo;
R⁴ is H, C₁-C₃ alkyl, C₁-C₃ haloalkyl, OR³, N(R⁸)₂ or halo;
R⁵ is H, C₁-C₃ alkyl, C₁-C₃ haloalkyl, OR³, N(Ra)₂ or halo;
R^{a} is independently H or C₁-C₃ alkyl; and
R³ is H, C₁-C₃ haloalkyl or C₁-C₃ alkyl;
n is 0, 1, 2 or 3; and
in is 0, 1, 2 or 3.

10. The compound of claim 8, wherein
Y is C and X is N;
R¹ is Cl;
R⁴ is H, methyl or halo;
R⁵ is H, methyl or halo;
n is 0 or 1; and
m is 0 or 1.

11. The compound of claim 1, wherein the compound is selected from the group consisting of
(S)-3-((2-(([1,1′-biphenyl]-3-yloxy)methyl)morpholino)sulfonyl)-5-chloro-1H-indole-2-carboxamide;
(S)-5-chloro-3-((2-((3-(pyridin-4-yl)phenoxy)methyl)morpholino)sulfonyl)-1H-indole-2-carboxamide;
(S)-5-chloro-3-((2-((3-(2-fluoropyridin-3-yl)phenoxy)methyl)morpholino)sulfonyl)-1H-indole-2-carboxamide;
(S)-5-chloro-3-((2-((3-(6-methylpyridin-3-yl)phenoxy)methyl)morpholino)sulfonyl)-1H-indole-2-carboxamide;
(S)-5-chloro-3-((2-((3-(6-(dimethylamino)pyridin-3-yl)phenoxy)methyl)morpholino)sulfonyl)-1H-indole-2-carboxamide;
(S)-5-chloro-3-((2-((3-(6-(trifluoromethyl)pyridin-3-yl)phenoxy)methyl)morpholino)sulfonyl)-1H-indole-2-carboxamide;
(S)-5-chloro-3-((2-((3-(6-methoxypyridin-3-yl)phenoxy)methyl)morpholino)sulfonyl)-1H-indole-2-carboxamide;
(S)-5-chloro-3-((2-((3-(6-fluoropyridin-3-yl)phenoxy)methyl)morpholino)sulfonyl)-1H-indole-2-carboxamide;
(S)-5-chloro-3-((2-(((2′-fluoro-[1,1′-biphenyl]-4-yl)oxy)methyl)morpholino)sulfonyl)-1H-indole-2-carboxamide;
(S)-5-chloro-3((2-(((3′-(dimethylamino)-[1,1′-biphenyl]-3-yl)oxy)methyl)morpholino)sulfonyl)-1H-indole-2-carboxamide;
(S)-5-chloro-3-((2-(((2′-methyl-[1,1′-biphenyl]-3-yl)oxy)methyl)morpholino)sulfonyl)-1H-indole-2-carboxamide;
(S)-5-chloro-3-((2-(((2′-methoxy-[1,1′-biphenyl]-4-yl)oxy)methyl)morpholino)sulfonyl)-1H-indole-2-carboxamide;
(S)-5-chloro-3-((2-((4-(2-methoxypyridin-3-yl)phenoxy)methyl)morpholino)sulfonyl)-1H-indole-2-carboxamide;
(S)-5-chloro-3-((2-(((4′-fluoro-[1,1′-biphenyl]-3-yl)oxy)methyl)morpholino)sulfonyl)-1H-indole-2-carboxamide;
(S)-5-chloro-3-((2-(((3′-fluoro-[1,1′-biphenyl]-3-yl)oxy)methyl)morpholino)sulfonyl)-1H-indole-2-carboxamide;
(S)-5-chloro-3-((2-((3-(2-fluoropyridin-4-yl)phenoxy)methyl)morpholino)sulfonyl)-1H-indole-2-carboxamide;
(S)-5-chloro-3-((2-(((3′-methyl-[1,1′-biphenyl]-3-yl)oxy)methyl)morpholino)sulfonyl)-1H-indole-2-carboxamide;
(S)-5-chloro-3-((2-((3-(pyridin-2-yl)phenoxy)methyl)morpholino)sulfonyl)-1H-indole-2-carboxamide;
(S)-5-chloro-3-((2-((3-(2-methoxypyrimidin-5-yl)phenoxy)methyl)morpholino)sulfonyl)-1H-indole-2-carboxamide;
(S)-5-chloro-3-((2-((3-(6-ethoxypyridin-3-yl)phenoxy)methyl)morpholino)sulfonyl)-1H-indole-2-carboxamide;
(S)-5-chloro-3-((2-((3-(2-methoxypyridin-4-yl)phenoxy)methyl)morpholino)sulfonyl)-1H-indole-2-carboxamide;
(S)-5-chloro-3-((2-((3-(2-methylpyridin-4-yl)phenoxy)methyl)morpholino)sulfonyl)-1H-indole-2-carboxamide;
(S)-5-chloro-3-((2-(((6-methyl-[1,1′-biphenyl]-3-yl)oxy)methyl)morpholino)sulfonyl)-1H-indole-2-carboxamide;
(S)-5-chloro-3-((2-(((2′-fluoro-6-methyl-[1,1′-biphenyl]-3-yl)oxy)methyl)morpholino)sulfonyl)-1H-indole-2-carboxamide;
(S)-5-chloro-3-((2-((3-(2-fluoropyridin-3-yl)-4-methylphenoxy)methyl)morpholino)sulfonyl)-1H-indole-2-carboxamide;
(S)-5-chloro-3-((2-(((2′-methoxy-6-methyl-[1,1′-biphenyl]-3-yl)oxy)methyl)morpholino)sulfonyl)-1H-indole-2-carboxamide;
(S)-5-chloro-3-((2-(((2′,6-dimethyl-[1,1′-biphenyl]-3-yl)oxy)methyl)morpholino)sulfonyl)-1H-indole-2-carboxamide;
(S)-5-chloro-3-((2-((3-(6-methoxypyridin-3-yl)-4-methylphenoxy)methyl)morpholino)sulfonyl)-1H-indole-2-carboxamide;
(S)-5-chloro-3-((2-((4-fluoro-3-(pyridin-3-yl)phenoxy)methyl)morpholino)sulfonyl)-1H-indole-2-carboxamide;
(S)-5-chloro-3-((2-((4-fluoro-3-(pyrimidin-5-yl)phenoxy)methyl)morpholino)sulfonyl)-1H-indole-2-carboxamide;
(S)-5-chloro-3-((2-((4-fluoro-3-(pyridin-4-yl)phenoxy)methyl)morpholino)sulfonyl)-1H-indole-2-carboxamide; (S)-5-chloro-3-((2-((4-fluoro-3-(6-methoxypyridin-3-yl)phenoxy)methyl)morpholino)sulfonyl)-1H-indole-2-carboxamide;
(S)-5-chloro-3-((2-((4-fluoro-3-(2-methylpyridin-4-yl)phenoxy)methyl)morpholino)sulfonyl)-1H-indole-2-carboxamide;
(S)-5-chloro-3-((2-((4-(pyridin-3-yl)phenoxy)methyl)morpholino)sulfonyl)-1H-indole-2-carboxamide;
(S)-5-chloro-3-((2-((4-(pyridin-4-yl)phenoxy)methyl)morpholino)sulfonyl)-1H-indole-2-carboxamide;
(S)-3-((2-(([1,1′-biphenyl]-4-yloxy)methyl)morpholino)sulfonyl)-5-chloro-1H-indole-2-carboxamide;
(S)-5-chloro-3-((2-((4-(6-(dimethylamino)pyridin-3-yl)phenoxy)methyl)morpholino)sulfonyl)-1H-indole-2-carboxamide;
(S)-5-chloro-3-((2-((4-(6-(trifluoromethyl)pyridin-3-yl)phenoxy)methyl)morpholino)sulfonyl)-1H-indole-2-carboxamide;
(S)-5-chloro-3-((2-(((2′-methoxy-[1,1′-biphenyl]-4-yl)oxy)methyl)morpholino)sulfonyl)-1H-indole-2-carboxamide;
(S)-5-chloro-3-((2-((4-(6-methoxypyridin-3-yl)phenoxy)methyl)morpholino)sulfonyl)-1H-indole-2-carboxamide;
(S)-5-chloro-3-((2-(((2′-(trifluoromethyl)-[1,1′-biphenyl]-4-yl)oxy)methyl)morpholino)sulfonyl)-1H-indole-2-carboxamide;
(S)-5-chloro-3-((2-(((4′-methoxy-[1,1′-biphenyl]-4-yl)oxy)methyl)morpholino)sulfonyl)-1H-indole-2-carboxamide;
(S)-5-chloro-3-((2-(((2′-(trifluoromethoxy)-[1,1′-biphenyl]-4-yl)oxy)methyl)morpholino)sulfonyl)-1H-indole-2-carboxamide;
(S)-5-chloro-3-((2-(((2′-methyl-[1,1′-biphenyl]-4-yl)oxy)methyl)morpholino)sulfonyl)-1H-indole-2-carboxamide;
(S)-5-chloro-3-((2-((4-(2-fluoropyridin-4-yl)phenoxy)methyl)morpholino)sulfonyl)-1H-indole-2-carboxamide;
(S)-5-chloro-3-((2-((4-(6-fluoropyridin-3-yl)phenoxy)methyl)morpholino)sulfonyl)-1H-indole-2-carboxamide;
(S)-5-chloro-3-((2-((4-(2-methoxypyridin-4-yl)phenoxy)methyl)morpholino)sulfonyl)-1H-indole-2-carboxamide;
(S)-5-chloro-3-((2-((4-(pyrimidin-5-yl)phenoxy)methyl)morpholino)sulfonyl)-1H-indole-2-carboxamide;
(S)-5-chloro-3-((2-((4-(2-fluoropyridin-3-yl)phenoxy)methyl)morpholino)sulfonyl)-1H-indole-2-carboxamide;
(S)-5-chloro-3-((2-((4-(2-methylpyridin-3-yl)phenoxy)methyl)morpholino)sulfonyl)-1H-indole-2-carboxamide;
(S)-5-chloro-3-((2-((4-(2-methylpyridin-4-yl)phenoxy)methyl)morpholino)sulfonyl)-1H-indole-2-carboxamide;
(S)-5-chloro-3-((2-((4-(6-methylpyridin-3-yl)phenoxy)methyl)morpholino)sulfonyl)-1H-indole-2-carboxamide;
(S)-5-chloro-3-((2-((4-(pyrimidin-2-yl)phenoxy)methyl)morpholino)sulfonyl)-1H-indole-2-carboxamide;
(S)-3-((2-(([1,1′-biphenyl]-2-yloxy)methyl)morpholino)sulfonyl)-5-chloro-1H-indole-2-carboxamide;
(S)-5-chloro-3-((2-((2-(pyridin-3-yl)phenoxy)methyl)morpholino)sulfonyl)-1H-indole-2-carboxamide;
(S)-5-chloro-3-((2-((2-(pyridin-4-yl)phenoxy)methyl)morpholino)sulfonyl)-1H-indole-2-carboxamide;
(S)-5-chloro-3-((2-((2-(2-fluoropyridin-3-yl)phenoxy)methyl)morpholino)sulfonyl)-1H-indole-2-carboxamide;
(S)-5-chloro-3-((2-(((2′-methoxy-[1,1′-biphenyl]-2-yl)oxy)methyl)morpholino)sulfonyl)-1H-indole-2-carboxamide;
(S)-5-chloro-3-((2((2-(6-fluoropyridin-3-yl)phenoxy)methyl)morpholino)sulfonyl)-1H-indole-2-carboxamide;
(S)-5-chloro-3-((2-((2-(pyrimidin-5-yl)phenoxy)methyl)morpholino)sulfonyl)-1H-indole-2-carboxamide;
(S)-5-chloro-3-((2-((2-(6-methoxypyridin-3-yl)phenoxy)methyl)morpholino)sulfonyl)-1H-indole-2-carboxamide;
(S)-5-chloro-3-((2-(((2′-fluoro-[1,1′-biphenyl]-2-yl)oxy)methyl)morpholino)sulfonyl)-1H-indole-2-carboxamide;
(S)-5-chloro-3-((2-((2-(2-methylpyridin-3-yl)phenoxy)methyl)morpholino)sulfonyl)-1H-indole-2-carboxamide;
(S)-5-chloro-3-((2-(((3′-fluoro-[1,1′-biphenyl]-2-yl)oxy)methyl)morpholino)sulfonyl)-1H-indole-2-carboxamide;
(S)-5-chloro-3-((2-(((2′-fluoro-4′-(trifluoromethyl)-[1,1′-biphenyl]-2-yl)oxy)methyl)morpholino)sulfonyl)-1H-indole-2-carboxamide;
(S)-5-chloro-3-((2-((3-methyl-4-(pyridin-3-yl)phenoxy)methyl)morpholino)sulfonyl)-1H-indole-2-carboxamide;
(S)-5-chloro-3-((2-((3-methyl-4-(pyridin-4-yl)phenoxy)methyl)morpholino)sulfonyl)-1H-indole-2-carboxamide;
(S)-5-chloro-3-((2-((4-(2-fluoropyridin-3-yl)-3-methylphenoxy)methyl)morpholino)sulfonyl)-1H-indole-2-carboxamide;
(S)-5-chloro-3-((2-((4-(6-methoxypyridin-3-yl)-3-methylphenoxy)methyl)morpholino)sulfonyl)-1H-indole-2-carboxamide;
(S)-5-chloro-3-((2-((4-(2-methoxypyridin-4-yl)-3-methylphenoxy)methyl)morpholino)sulfonyl)-1H-indole-2-carboxamide;
(S)-5-chloro-3-((2-((3-methyl-4-(pyrimidin-5-yl)phenoxy)methyl)morpholino)sulfonyl)-1H-indole-2-carboxamide;
(S)-5-chloro-3-((2-((4-(2-fluoropyridin-4-yl)-3-methylphenoxy)methyl)morpholino)sulfonyl)-1H-indole-2-carboxamide;
(S)-5-chloro-3-((2-((4-(6-fluoropyridin-3-yl)-3-methylphenoxy)methyl)morpholino)sulfonyl)-1H-indole-2-carboxamide;
(S)-5-chloro-3-((2-(((5-(4-methoxyphenyl)pyridin-3-yl)oxy)methyl)morpholino)sulfonyl)-1H-indole-2-carboxamide;
(S)-5-chloro-3-((2-(((5-(2-fluoro-5-(trifluoromethyl)phenyl)pyridin-3-yl)oxy)methyl)morpholino)sulfonyl)-1H-indole-2-carboxamide;
(S)-5-chloro-3-((2-(((5-phenylpyridin-3-yl)oxy)methyl)morpholino)sulfonyl)-1H-indole-2-carboxamide;
(S)-5-chloro-3-((2-(((5-phenylpyrazin-2-yl)oxy)methyl)morpholino)sulfonyl)-1H-indole-2-carboxamide;
(S)-5-chloro-3-((2-(((5-(2-fluorophenyl)pyridin-3-yl)oxy)methyl)morpholino)sulfonyl)-1H-indole-2-carboxamide;
(S)-5-chloro-3-((2-(((6-phenylpyridin-3-yl)oxy)methyl)morpholino)sulfonyl)-1H-indole-2-carboxamide;
(S)-5-chloro-3-((2-(((6-phenylpyridin-2-yl)oxy)methyl)morpholino)sulfonyl)-1H-indole-2-carboxamide;
(S)-5-chloro-3-((2-(((5-phenylpyridin-2-yl)oxy)methyl)morpholino)sulfonyl)-1H-indole-2-carboxamide;
(S)-5-chloro-3-((2-((4-(2,6-dimethylpyridin-4-yl)phenoxy)methyl)morpholino)sulfonyl)-1H-indole-2-carboxamide;
(S)-5-chloro-3-((2-(((2′-chloro-[1,1′-biphenyl]-4-yl)oxy)methyl)morpholino)sulfonyl)-1H-indole-2-carboxamide;
(S)-5-chloro-3-((2-((4-(2-chloropyridin-4-yl)phenoxy)methyl)morpholino)sulfonyl)-1H-indole-2-carboxamide;
(S)-5-chloro-3-((2-((3-(2-chloropyridin-4-yl)phenoxy)methyl)morpholino)sulfonyl)-1H-indole-2-carboxamide;
(S)-5-chloro-3-((2-((3-(6-chloropyridin-3-yl)phenoxy)methyl)morpholino)sulfonyl)-1H-indole-2-carboxamide;
(S)-5-chloro-3-((2-((4-(2-chloropyridin-4-yl)-3-methylphenoxy)methyl)morpholino)sulfonyl)-1H-indole-2-carboxamide;
(S)-5-chloro-3-((2-((4-(6-chloropyridin-3-yl)-3-methylphenoxy)methyl)morpholino)sulfonyl)-1H-indole-2-carboxamide;
(S)-5-chloro-3-((2-((cinnolin-3-yloxy)methyl)morpholino)sulfonyl)-1H-indole-2-carboxamide;
(S)-5-chloro-3-((2-((quinolin-8-yloxy)methyl)morpholino)sulfonyl)-1H-indole-2-carboxamide;
(S)-5-chloro-3-((2-((quinazolin-4-yloxy)methyl)morpholino)sulfonyl)-1H-indole-2-carboxamide; and
(S)-5-chloro-3-((2-(((1-methyl-3-phenyl-1H-pyrazol-5-yl)oxy)methyl)morpholino)sulfonyl)-1H-indole-2-carboxamide;
stereoisomers thereof;
pharmaceutically acceptable salts thereof; and
pharmaceutically acceptable salts of the stereoisomers thereof.

12. A pharmaceutical composition comprising a therapeutically effective amount of the compound of claim 1 and a pharmaceutically acceptable carrier.

13. (canceled)

14. The pharmaceutical composition of claim 12, further comprising one or more therapeutic ingredients.

15. The compound of claim 6, wherein
Y is N and X is C or X and Y are both C;
R¹ is Cl;
R⁴ is H, methyl, methoxy or halo;
R⁵ is H, methyl, methoxy or halo;
n is 0 or 1; and
m is 0 or 1.

16. The compound of claim 9, wherein
Y is C and X is N;
R¹ is Cl;
R⁴ is H, methyl or halo;
R⁵ is H, methyl or halo;
n is 0 or 1; and
m is 0 or 1.

17. A method of treating cancer comprising administering a therapeutically effective amount of the compound of claim 1 to a person in need of treatment.
